(19) United States
(12) Patent Application Publication
(10) Pub. No.: US 2008/0181894 A1
(43) Pub. Date: Jul. 31, 2008

(54) Identification of Human Gene Sequences of Cancer Antigens Expressed in Metastatic Carcinoma Involved in Metastasis Formation, and Their Use in Cancer Diagnosis, Prognosis and Therapy

(76) Inventors: Sigrun Mink, Karlsruhe (DE); Joerg Mengwasser, Berlin (DE); Elke Martin, Karlsruhe (DE); Birgit Simgen, Karlsruhe (DE); Monika Raab, Ronneburg (DE); Sylvia Schwarz, Frankfurt/M (DE); Bernd Hentsch, Frankfurt/M (DE)

Correspondence Address: CERMAK KENEALY & VAIDYA LLP, 515 E. BRADDOCK RD, SUITE B, ALEXANDRIA, VA, 22314, US

(21) Appl. No.: 11/912,533

(22) PCT Filed: Apr. 21, 2006
(86) PCT No.: PCT/EP2006/003713

§ 371 Date: Oct. 25, 2007

(30) Foreign Application Priority Data
Apr. 26, 2005 (EP) 05103409.8

(51) Int. Cl. A61K 31/70; C12Q 1/68; G01N 33/574; C12Q 1/02; A61K 39/395; A61P 43/00; A61K 38/00; C12Q 1/04; C07H 21/00

(52) U.S. Cl. 424/138.1; 435/6; 435/7.23; 435/29; 435/34; 536/24.5; 514/44; 514/2

(57) ABSTRACT

The present invention relates to methods using newly identified cancer related polynucleotides and the polypeptides encoded by these polynucleotides. The invention further relates to the use of such “cancer antigens” for diagnosing cancer and cancer metastases. The invention relates to the use of these cancer antigens employing expression vectors, host cells, antibodies directed to such cancer antigens, and recombinant methods and synthetic methods for producing the same. Also provided are diagnostic and prognostic methods for detecting, treating, or preventing cancer, for suppressing tumor progression and minimal residual tumor disease, and therapeutic methods for treating such disorders. The invention further relates to screening methods for identifying agonists and antagonists of the cancer antigens of the invention. The present invention further relates to inhibiting the production and function of the polynucleotides and polypeptides of the present invention.

### BACKGROUND OF THE INVENTION

It has been widely accepted that carcinogenesis is a multistep process involving genetic and epigenetic changes that dysregulate molecular control of cell proliferation and differentiation (Balmain, 2003, Nat. Genet. 33, 238-244). The genetic changes can include activation of proto-oncogenes and/or the inactivation of tumor suppressor genes that can initiate tumorigenesis. Tumorprogression and Metastasis are also multi-stage processes by which tumor cells leave the site of a primary tumor, enter blood and lymph vessels, migrate to distant parts of the body and form novel foci of tumor growth. Metastasis is a major cause of mortality for cancer patients. Many studies on cancer metastasis have been conducted and several molecules participating in tumor cell invasion and metastasis have been identified and characterized. Among these molecules, some facilitate invasion and metastasis, e.g. laminin receptor, metalloproteinases, and CD44 (Hojilla, 2003, Br. J. Cancer 89, 1817-1821; Marhaba, 2004, J. Mol. Histol. 35, 211-231).

Despite use of a number of histochemical, genetic, and immunological markers, clinicians still have a difficult time predicting which tumors will progress and will finally metastasize to other organs, or whether a patient has already developed early metastasis. Some patients are in need of adjuvant therapy to prevent recurrence and metastasis and others are not. Distinguishing between these subpopulations of patients is not straightforward. There is therefore a need for new markers for distinguishing between tumors of differing metastatic potential and for new molecular targets and new therapeutic treatment options. In addition, such markers could be useful to monitor a potential anti-tumor response of a patient's body upon treatment with an anti-cancer drug.

Modern drug development typically involves the elucidation of the molecular mechanism underlying a disease or a condition, the identification of candidate target molecules and the evaluation of said target molecules. It is obvious that the identification of a candidate target molecule is essential to such process. With the sequencing of the human genome and publishing of respective sequence data, in principle, all of the coding nucleic acids of man are available. However, a serious limitation to this data is that typically no annotation of the function of said sequence is given. Furthermore, the mere knowledge of a coding nucleic acid sequence is not sufficient to predict the polypeptide's function in vivo.

In order to utilize such aforementioned new markers, it is required to identify the molecular basis of these markers based on their gene nucleotide and protein sequences. To define the profile of such genes whose expression is up-regulated during progression from a non metastasizing to metastatic cancer competence, initially rat tumor progression models were used for the identification of the markers presented in this invention. Here, instead of starting directly from human tumor material, it was chosen to analyze precisely defined clonal rodent tumor cell lines in a first differential gene sequence expression analysis. The utilization of such well characterized tumor cell lines offers the advantage that they often exhibit a reproducible metastatic or nonmetastatic phenotype that can be retested at any stage of the analysis. Moreover, tumor cell lines are accessible to genetic manipulation and functional tests in experimental animals. Rat tumor cells have the advantage of being able to be passaged in syngeneic animals, whereas human tumor cells have to be passaged in the rather artificial setting of an immunodeficient host. Furthermore, the cross species homology between rodent and human sequences creates the opportunity for the subsequent isolation of human homologues of such candidate tumor progression genes, hereafter referred to as “cancer antigens”, and evaluation of their expression in primary human tumor material.

For the above mentioned intended molecular comparison of gene expression differences, two rat carcinoma models were used. The first model represents a rat pancreatic adenocarcinoma model which comprises several clones that differ in their metastatic potential in vivo and have been derived from a common primary tumor (Matzku, 1983, Cancer Research 49, 1294-1299). For example, BSp73-1AS cells form primary tumors that do not metastasize, whereas BSp73-ASML cells are highly metastatic and, after s.c. injection into host animals, disseminate via the lymphatic system to finally colonize the lungs. The second system, the rat mammary adenocarcinoma cell system 13762NF (Neri, 1981, Int. J. Cancer 28, 731-738), is composed of a number of cell lines derived from a parental mammary tumor and its corresponding spontaneous lung and lymph node metastases. For example, the cell line MTPa has been reported to be nonmetastatic in vivo in syngeneic animals, whereas the related MTLY cells are highly metastatic, giving rise to multiple metastases in the lymph nodes and lungs (Neri, 1981, Int. J. Cancer 28, 731-738). These systems guarantee a high reproducibility of the cellular metastatic potentials and provide a reproducible and easy access to cellular material. Thus, a high standard of quality and quantity of the critical starting material is warranted. The metastatic and the non-metastatic material is highly related, a relationship which cannot be reached using human primary or secondary tumors or human tumor derived cell lines as frequently employed in other studies.

In order to identify gene sequences—cancer antigens—in these systems which are stronger expressed in cells displaying high metastatic potential in comparison to related cells with a lower metastatic potential, transcripts of the non-metastatic cell line were subtracted from those of the metastatic cells via the Subtractive Suppression Hybridization (SSH Analysis) (Nestl, 2001, Cancer Research 61, 1569-1577) technology. For this purpose, RNA was isolated from the metastatic (tester population) and non-metastatic cells (driver population), cDNA was then generated and digested to get smaller, suitably sized pieces of DNA. Tester cDNA was divided into two portions and each was ligated with a different adaptor. Each tester sample was then hybridized with an excess of driver cDNA. Only DNA fragments specifically present in the tester sample (derived from the metastatic cells) remained single stranded. The primary hybridization samples were then mixed and hybridized again. Now, only the remaining equalized and subtracted single strand tester cDNAs are able to reassociate and form hybrids with two different adaptors. Those fragments with two different adaptor ends could then be amplified by PCR and transferred into suitable vector systems for further analysis. Therefore, only the transcripts specifically expressed in metastatic cells are amplified whereas the amplification of transcripts present in both populations is suppressed (Diatschenko, 1996, Proc. Natl. Acad. Sci. 93, 6025-6030).

Using this analysis, 981 differentially expressed cDNA clones from these rat systems were isolated, which after analysis using sequence blast and clustering analysis bioinformatics tools equated to 229 individual rat sequences. Of those, 189 could subsequently be transferred to human sequences utilizing human gene sequence data banks and advanced bioinformatics analysis. Of these 189 gene sequences, 144 represented human proteins of known function, and 45 coded for human proteins of unknown function or hypothetical proteins.

To further characterize these sequences in respect to their biological connection to the process of tumor progression and metastasis formation, and to verify their suitability as cancer antigens or as metastasis markers, several additional analytical examinations were applied. Initially, all sequences of which a connection to metastasis formation or tumor progression has previously already been reported were sorted out. Secondly, the expression of the remaining gene sequences was analyzed in human tumor samples, and thirdly, the functional involvement of these sequences in cellular metastatic processes was analyzed by (i) overexpression of the gene sequences, and (ii) by RNA interference studies in suitable test systems. This analytical process revealed 9 previously not described new cancer antigens or metastasis markers which are useful as diagnostic tools or which may serve as new target structures to create new therapeutic treatment options for cancer patients, and which are one subject of this invention.

This invention relates to these sequences and their role in cellular process of increased metastasizing potential since their expression is found to be increased parallel to the increase in this metastasising potential. Thus, these gene sequences and the proteins encoded thereof may alone or in combination of two or more of these sequences contribute to the establishment of, or the progression to a more metastatic phenotype. With this respect, the pro-metastatic activities of a given sequence or the respectively encoded polypeptide may be enhanced when these activities are combined with the pro-metastatic activities of another sequence or polypeptide encoded thereof. Thus, the acquisition of pro-metastatic activities through enhanced expression of such individual sequences and polypeptides must therefore be regarded as part of a process in which a cell step wise acquires an increasing metastatic phenotype, whereas such a single step is defined by the acquisition of the upregulated expression of one of these sequences. This implies that these sequences are functionally linked to each other by each adding one step to the process of cellular metastatic potential, and these sequences should therefore be regarded as all being part of the same process, and therefore the same underlying invention which is presented herein.

A first aspect of the present invention is a method for diagnosing a disease or condition, or a susceptibility to a disease or condition, comprising the step of determining the expression, activity or mutations of at least one polynucleotide or expression product thereof in a biological sample from a (first) subject, wherein said at least one polynucleotide comprises
(i) a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9 and the corresponding RNA sequences,
(ii) a sequence complementary to any one of the sequences under (i), or
(iii) a variant sequence of any one of the sequences under (i) or (ii).

The subject from which the biological sample was obtained may be a patient having the disease or condition, or an individual not affected by the disease or condition. In the latter case, the subject may be an individual suspected of having the disease or condition. Usually, the subject is a human.

The biological sample may be derived from or contain a body liquid obtained from said subject, for example blood or cerebrospinal fluid. In a preferred embodiment, the biological sample contains tissue material obtained through biopsy. The tissue may be a tissue affected by the disease or condition, e.g. a solid tumor. A tissue affected by the disease or condition is a tissue which differs from the corresponding tissue from a healthy individual. The difference may be a difference in morphology, histology, gene expression, response to treatment, protein composition etc.

Usually, the sample has been processed to be in a condition suitable for the method of determining the expression, activity or mutations as detailed infra. The processing may include dilution, concentration, homogenization, extraction, precipitation, fixation, washing and/or permeabilization, etc. The processing may also include reverse transcription and/or amplification of nucleic acids present in the sample.

The method of the invention may comprise only steps which are carried out in vitro. In that case, the step of obtaining the tissue material from the subject's body is not encompassed by the present invention. In another embodiment, the method further comprises the step of obtaining the biological sample from the subject's body.

The method comprises the step of determining the expression, activity or mutations of at least one polynucleotide or expression product thereof in a biological sample. The phrase “determining the expression” as used herein preferably means “determining the expression level”. The expression or expression level correlates with the amount of polynucleotide or expression product thereof in the sample. The phrase “determining the expression of polynucleotide or expression product in the biological sample” includes or consists of determining the presence and/or amount of said at least one polynucleotide or expression product thereof. As used herein, the phrase “determining the mutations” means determining the presence or absence of one or more mutations in the nucleotide sequence of said at least one polynucleotide in said biological sample. It is preferred that mutations with respect to any one of the sequences SEQ ID NO:1 through 9 are determined.

The term “polynucleotide(s)” generally refers to any polyribonucleotide or polydeoxyribonucleotide that may be RNA or DNA. The polynucleotide may be single- or double-stranded. The polynucleotide in accordance with the diagnostic method of this invention may have a sequence as shown in any one of SEQ ID NO:1, SEQ ID NO:2, SEQ ID NO:3, SEQ ID NO:4, SEQ ID NO:5, SEQ ID NO:6, SEQ ID NO:7, SEQ ID NO:8 and SEQ ID NO:9. In addition, the polynucleotide may have a sequence which is a variant of these sequences. The variant may be a sequence having one or more additions, substitutions, and/or deletions of one or more nucleotides such as an allelic variant or single nucleotide polymorphisms of the above sequences. The variant may have an identity of at least 80%, preferably of at least 85%, more preferably of at least 90%, even more preferably of at least 95%, most preferably of at least 99% to any one of the sequences SEQ ID NO:1, SEQ ID NO:2, SEQ ID NO:3, SEQ ID NO:4, SEQ ID NO:5, SEQ ID NO:6, SEQ ID NO:7, SEQ ID NO:8 and SEQ ID NO:9. The percent identity or conservation may be determined by the algorithm of Wilbur and Lipman, Proc. Natl. Acad. Sci. USA 80; 726-730 (1983) which is embodied in the MegAlign program (DNA Star), using a k-tuple of 3 and a gap penalty of 3. Alternatively the algorithm of Myers and Miller, CABIOS (1989), which is embodied in the ALIGN program (version 2.0) or its equivalent, using a gap length penalty of 12 and a gap penalty of 3 where such parameters are required. All other parameters are set to their default positions. Access to ALIGN is readily available (see, e.g., http://www2.igh.cnrs.fr/bin/align-guess.cgi on the Internet).

The variant may be a polynucleotide which hybridizes to any one of the sequences SEQ ID NO:1 through 9, preferably under stringent conditions. A specific example of stringent hybridization conditions is incubation at 42° C. for 16 hours in a solution comprising: 50% formamide, 5×SSC (150 mM NaCl, 15 mM trisodium citrate), 50 mM sodium phosphate (pH7.6), 5× Denhardt's solution, 10% dextran sulfate, and 20 μg/ml of denatured, sheared salmon sperm DNA, followed by washing the hybridization support in 0.1×SSC at about 65° C. Hybridization and wash conditions are well known and exemplified in Sambrook, et al., Molecular Cloning: A Laboratory Manual, Second Edition, Cold Spring Harbor, N.Y., (1989), particularly Chapter 11 therein. Alternative hybridization conditions are described infra with respect to solid supports.

In the variant 1 to 20, preferably 1 to 10, more preferably 1 to 5, most preferably 1, 2 or 3 nucleotides may be added, substituted or inserted with respect to any one of the sequences as shown in SEQ ID NO:1, SEQ ID NO:2, SEQ ID NO:3, SEQ ID NO:4, SEQ ID NO:5, SEQ ID NO:6, SEQ ID NO:7, SEQ ID NO:8 and SEQ ID NO:9. The variants further include fragments of SEQ ID NO:1 through 9. The fragments may comprise at least 100, preferably at least 500, more preferably at least 1000 contiguous nucleotides of any one of SEQ ID NO:1 through 9. Most preferably the fragment has a length such that less than 100, or less than 50, or less than 25 nucleotides are missing with respect to any one of SEQ ID NO:1 through 9.

Alternatively, the polynucleotide may have the corresponding RNA sequence. The sequence of the polynucleotide may also be complementary to any one of the above sequences.

Preferably, the polynucleotide in accordance the diagnostic method of this invention comprises a sequence encoding a polypeptide having a sequence selected from the group consisting of SEQ ID NO:10, SEQ ID NO:11, SEQ ID NO:12, SEQ ID NO:13, SEQ ID NO:14, SEQ ID NO:15, SEQ ID NO:16, SEQ ID NO:17 and SEQ ID NO:18.

The expression product of said polynucleotide usually is a polypeptide encoded by any one of the above polynucleotides. Preferably, the polypeptide comprises a sequence selected from the group consisting of SEQ ID NO:10, SEQ ID NO:11, SEQ ID NO:12, SEQ ID NO:13, SEQ ID NO:14, SEQ ID NO:15, SEQ ID NO:16, SEQ ID NO:17 and SEQ ID NO:18. The polypeptide may be a variant of any one of SEQ ID NO:10-18. For example, the amino acid sequence of the polypeptide may have an identity of at least 80%, preferably of at least 85%, more preferably of at least 90%, even more preferably of at least 95%, most preferably of at least 98% to any one of the sequences SEQ ID NO:10-18. The identity is to be understood as identity over the entire length of the polypeptide. The percent identity or conservation may be determined by the algorithm of Wilbur and Lipman, Proc. Natl. Acad. Sci. USA 80; 726-730 (1983) which is embodied in the MegAlign program (DNA Star), using a k-tuple of 3 and a gap penalty of 3. Alternatively the algorithm of Myers and Miller, CABIOS (1989), which is embodied in the ALIGN program (version 2.0) or its equivalent, using a gap length penalty of 12 and a gap penalty of 3 where such parameters are required. All other parameters are set to their default positions. Access to ALIGN is readily available (see, e.g., http://www2.iqh.cnrs.fr/bin/align-guess.cgi on the Internet).

In the variant 1 to 10, preferably 1 to 5, more preferably 1 to 4, most preferably 1, 2 or 3 amino acids may be added, substituted or inserted with respect to any one of the sequences as shown in SEQ ID NO:10 through 18. The variants further include fragments of SEQ ID NO:10 through 18. The fragments may comprise at least 50, preferably at least 100, more preferably at least 500 contiguous amino acids of any one of SEQ ID NO:10 through 18. Most preferably the fragment has a length such that less than 50, or less than 30, or less than 15 amino acids are missing with respect to any one of SEQ ID NO:10 through 18.

In some embodiments, the variant polynucleotides and/or the polypeptides they encode retain at least one activity or function of the unmodified polynucleotide and/or the polypeptide, such as hybridization, antibody binding, etc.

In one embodiment, the method comprises the use of nucleic acid hybridization technology for determining the amount or presence of the polynucleotide in the sample, or for determining the mutations in the polynucleotide. Hybridization methods for nucleic acids are well known to those of ordinary skill in the art (see, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York).

According to the invention, standard hybridization techniques of microarray technology may be utilized to assess polynucleotide expression. Microarray technology, which is also known as DNA chip technology, gene chip technology, and solid-phase nucleic acid array technology, is well known to the skilled person and is based on, but not limited to, obtaining an array of identified nucleic acid probes on a fixed support, labeling target molecules with reporter molecules (e.g., radioactive, chemiluminescent, or fluorescent tags), hybridizing target nucleic acids to the probes, and evaluating target-probe hybridization. A probe with a nucleic acid sequence that perfectly matches the target sequence will, in general, result in detection of a stronger reporter-molecule signal than will probes with less perfect matches. Many components and techniques utilized in nucleic acid microarray technology are presented in “The Chipping Forecast”, Nature Genetics, Vol. 21, January 1999.

According to the present invention, microarray supports may include but are not limited to glass, silica, aluminosilicates, borosilicates, plastics, metal oxides, nitrocellulose, or nylon. The use of a glass support is preferred. According to the invention, probes are selected from the group of polynucleotides including, but not limited to: DNA, genomic DNA, cDNA, and oligonucleotides; and may be natural or synthetic. Oligonucleotide probes preferably are 20 to 25-mer oligonucleotides and DNA/cDNA probes preferably are 500 to 5000 bases in length, although other lengths may be used. Appropriate probe length may be determined by the skilled person by known procedures. Probes may be purified to remove contaminants using standard methods known to those of ordinary skill in the art such as gel filtration or precipitation. Accordingly, the polynucleotide immobilized to the solid support is preferably an isolated polynucleotide. The term “isolated” polynucleotide refers to a polynucleotide that is substantially free from other nucleic acid sequences, such as and not limited to other chromosomal and extrachromosomal DNA and RNA. Isolated polynucleotides may be purified from a host cell. Conventional nucleic acid purification methods known to skilled artisans may be used to obtain isolated polynucleotides. The term also includes recombinant polynucleotides and chemically synthesized polynucleotides.

In one embodiment, probes are synthesized directly on the support in a predetermined grid pattern using methods such as light-directed chemical synthesis, photochemical deprotection, or delivery of nucleotide precursors to the support and subsequent probe production. In embodiments of the invention one or more control polynucleotides are attached to the support. Control polynucleotides may include but are not limited to cDNA of genes such as housekeeping genes or fragments thereof.

The solid support comprises at least one polynucleotide immobilized on or attached to its surface, wherein said polynucleotide hybridizes with a polynucleotide as described supra, preferably under stringent conditions. Suitable hybridization conditions are for example described in the manufacturer's instructions of “DIG Easy Hyb Granules” (Roche Diagnostics GmbH, Germany, Cat. No. 1796895). These instructions are incorporated herein by reference. The hybridization conditions described in the following protocol may be used:
Hybridizations are carried out using DIG Easy Hyb buffer (Roche Diagnostics, Cat. No. 1796895).
Ten microliters of hybridization solution with probe is placed on the microarray and a coverslip carefully applied.
The slide is placed in a hybridization chamber and incubated for 16 h incubation at 42° C.
The coverslips are removed in a container with 2×SSC+0.1% SDS and the microarrays are washed for 15 min in 2×SSC+0.1% SDS at 42° C. followed by a 5 min wash in 0.1×SSC+0.1% SDS at 25° C. followed by two short washes in 0.1×SSC and 0.01×SSC at 25° C., respectively.
The microarrays are dried by centrifugation and can be stored at 4° C.

Preferably, the polynucleotide immobilized on the solid support has a sequence as shown in any one of SEQ ID NO:1 through 9; or a complement thereof; or a fragment thereof.

In one embodiment, preferred probes are sets of two or more of the nucleic acid molecules as defined. In a specific embodiment, at least 9 different isolated polynucleotides are immobilized on said solid support, and said 9 different isolated polynucleotides have the nucleotide sequences as shown in SEQ ID NO:1, SEQ ID NO:2, SEQ ID NO:3, SEQ ID NO:4, SEQ ID NO:5, SEQ ID NO:6, SEQ ID NO:7, SEQ ID NO:8 and SEQ ID NO:9, respectively, or the corresponding complementary sequences, or fragments thereof.

In another embodiment, at least 20 or at least 50 or at least 75 different isolated polynucleotides selected from the polynucleotides listed in FIG. 1 are immobilized on said solid support. In a specific embodiment, at least 89 different isolated polynucleotides are immobilized on said solid support, and said at least 89 isolated polynucleotides have the nucleotide sequences as outlined in FIG. 1. The nucleotide sequences of the polynucleotides as outlined in FIG. 1 are defined by their name and/or accession number and are incorporated herein by reference.

In another embodiment, the method comprises utilizing an antibody directed against a polypeptide described hereinabove. Preferably, the polypeptide is selected from the group consisting of SEQ ID NO:10, SEQ ID NO:11, SEQ ID NO:12, SEQ ID NO:13, SEQ ID NO:14, SEQ ID NO:15, SEQ ID NO:16, SEQ ID NO:17 and SEQ ID NO:18. The antibody may be polyclonal or monoclonal, with monoclonal antibodies being preferred. The antibody is preferably immunospecific for any one of the above polypeptides. The antibodies can be used to detect the polypeptide by any standard immunoassay technique including ELISA, immunoblotting (Western blotting), immunoprecipitation, BIACORE technology and the like, as will be appreciated by one of ordinary skill in the art.

The method of the invention usually further comprises the step of comparing said expression or activity determined as described supra and the expression or activity of said polynucleotide or expression product thereof in a second sample which was obtained from tissue which is not affected by said disease. For example, an increased expression or activity in said first sample compared to the expression or activity in said second sample may be diagnostic of the disease. The second sample may be derived from a second subject which is not affected by the disease. Alternatively, the second sample may be derived from the first subject, but from a different tissue than the first sample.

The disease may be a tumor disease or cancer. Preferably, the disease is any one of the following diseases and conditions: estrogen receptor-dependent breast cancer, estrogen receptor-independent breast cancer, hormone receptor-dependent prostate cancer, hormone receptor-independent prostate cancer, brain cancer, renal cancer, colon cancer, colorectal cancer, pancreatic cancer, bladder cancer, esophageal cancer, stomach cancer, genitourinary cancer, gastrointestinal cancer, uterine cancer, ovarian cancer, astrocytomas, gliomas, skin cancer, squamous cell carcinoma, Keratoakantoma, Bowen disease, cutaneous T-Cell Lymphoma, melanoma, basal cell carcinoma, actinic keratosis, sarcomas, Kaposi's sarcoma, osteosarcoma, head and neck cancer, small cell lung carcinoma, non-small cell lung carcinoma, leukemias, lymphomas, or other blood cell cancers, ichtiosis, acne, acne vulgaris, thyroid resistance syndrome, diabetes, thalassemia, cirrhosis, protozoal infection, rheumatoid arthritis, rheumatoid spondylitis, all forms of rheumatism, osteoarthritis, gouty arthritis, multiple sclerosis, insulin dependent diabetes mellitus, non-insulin dependent diabetes, asthma, rhinitis, uveithis, lupus erythematoidis, ulcerative colitis, Morbus Crohn, inflammatory bowel disease, chronic diarrhea, psoriasis, atopic dermatitis, bone disease, fibroproliferative disorders, atherosclerosis, aplastic anemia, DiGeorge syndrome, Graves' disease, epilepsia, status epilepticus, alzheimer's disease, depression, schizophrenia, schizoaffective disorder, mania, stroke, mood-incongruent psychotic symptoms, bipolar disorder, affective disorders, meningitis, muscular dystrophy, multiple sclerosis, agitation, cardiac hypertrophy, heart failure, reperfusion injury and obesity.

Most preferably, the disease is minimal residual disease or tumor metastasis.

The genes identified herein permit, inter alia, rapid screening of biological samples by nucleic acid microarray hybridization or protein expression technology to determine the expression of the specific genes and thereby to predict the outcome of the disease. Such screening is beneficial, for example, in selecting the course of treatment to provide to the patient, and to monitor the efficacy of a treatment.

Another aspect of this invention is a method for identifying compounds which modulate the expression or activity of any of the polynucleotides or expression products thereof as defined in any one of claims 1 to 3, comprising
(a) contacting a candidate compound with cells which express said polynucleotide or a polypeptide encoded thereby, or with cell membranes comprising said polypeptide, or respond to said polypeptide,
(b) determining the effect of said candidate compound on the expression, activity, cellular localization or structural condition of said polynucleotide or polypeptide, or determining a functional response of said cells.

The step of determining the effect may comprise comparing said expression, activity, cellular localization or structural condition of said polynucleotide or polypeptide with the expression, activity, cellular localization or structural condition of said polynucleotide or polypeptide in cells which were not contacted with the candidate compound. The method may further comprise comparing the viability of the cells which were contacted with the candidate compound and the viability of cells which were not contacted with the candidate compound.

The candidate compound may be selected if the expression of said polynucleotide or polypeptide in the cells which were contacted with the candidate compound is lower than in the cells which were not contacted with the candidate compound. In such case, the compound is capable of suppressing the expression of the polynucleotide or expression product thereof. One may further compare the viability of the cells which were contacted with the candidate compound and the viability of cells which were not contacted with the candidate compound.

The invention further concerns a compound identified by the above-described method, wherein said compound is a compound which antagonizes or agonizes any one of the polynuleotides or expression products thereof as defined in this application. Such compounds include but are not limited to antisense nucleic acid molecules capable of suppressing the expression of any one of the polynucleotides or expression products thereof as defined herein.

Yet another aspect of the invention is a solid support on which at least one isolated polynucleotide is immobilized, wherein said isolated polynucleotide has
(i) a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and fragments thereof;
(ii) a sequence complementary to any one of the sequences under (i); or
(iii) a sequence which is an allelic variant of any one of the sequences under (i) or (ii).

The solid support preferably has the form of a microarray or DNA chip. Other preferred embodiments of the solid support have been described hereinabove in connection with the diagnostic methods of the invention.

Yet another aspect of the invention is the use of a polynucleotide or polypeptide as defined herein for the diagnostic method, or of a compound identified by the screening method described above, in the manufacture of a medicament for the treatment or prevention of a disease associated with increased activity or expression of a polynucleotide or polypeptide as defined herein.

Diagnostic tools based on the newly identified cancer antigens are another subject of this invention, and include test systems to analyze expression of these sequences in tumors to predict the tumor's potential to progress and to develop metastasis. In addition, these tools can be used to examine a patient's body for the presence of micrometastases or minimal residual disease which may lead to improved decisions on further treatment modalities. In this respect, a test system applied could consist of cDNAs, comprising, e.g., the cancer antigen sequences, which are contained on a carrier system, such as being spotted on, e.g., glass slides (gene or cDNA chip) which subsequently would be analysed utilizing fluorescence labelled RNA samples—derived from patients—that are hybridized to these chips to investigate the expression patterns of several metastasis markers—including the cancer antigens—at the same time.

Therefore, the present invention relates to methods for the diagnosis or screening of a subject in need, e.g., a patient suffering from a disease, e.g., but not limited to cancer, which correlates with the expression of at least one of the cancer antigens of this invention, to test whether the subject displays an enhanced activity or expression of a polynucleotide or polypeptide. Such investigations could, e.g., give information about the presence of the metastatic potential of a patient's tumor cells, or whether a patient's body harbors minimal residual tumor disease. These investigations may comprise nucleic acid technologies, such as hybridisation methods using hybridisation samples derived from patient's normal or diseased tissues. Also, such processes may be useful to draw prognostic conclusions about about a patient's disease, or about a patient's response to a therapeutic treatment by monitoring of the clinical effectiveness of the treatment, and the correlation of the expression or activity of a cancer antigen (polynucleotide or polypeptide) of this invention.

Furthermore, since the genes or gene products coding for the cancer antigens of this invention could be causally involved in the progression of tumor diseases, these gene sequences or gene products encoded by those, may represent new target structures for the development of new drugs, including but not limited to anti-cancer drugs, and the subsequent therapeutic treatment of patients with these drugs.

Therefore, this invention also comprises methods for the treatment of a subject having the need to inhibit the activity or expression of a polynucleotide or polypeptide presented herein. Such treatment could comprise one or more of the following steps targeting the expression or function of a polynucleotide or polypeptide:
(a) administering to the subject a therapeutically effective amount of a compound which causes a decrease in the expression of a polynucleotide,
(b) administering to the subject a therapeutically effective amount of an antagonist to said polypeptide,
(c) administering to the subject a therapeutically effective amount of an agonist to said polypeptide,
(d) administering to the subject a nucleic acid molecule that inhibits the expression of the nucleotide sequence encoding said polypeptide,
(e) administering to the subject a polynucleotide or a nucleotide sequence complementary to said nucleotide sequence in a form so as to effect production of said thereof encoded polypeptide activity,
(f) administering to the subject a therapeutically effective amount of a polypeptide that competes with said polypeptide for its ligand, substrate, or receptor,
(g) administering to the subject a therapeutically effective amount of an antibody directed against said polypeptide.

This invention also comprises methods for the expression, production and/or functional analysis of specific polynucleotides and polypeptides. For this purpose, a polynucleotide covered by this invention should be defined as comprising a nucleotide sequence that has at least 80% identity over its entire length to any of the polynucleotide sequences described herein. More preferably, the identity is larger than 90%, and even more preferably, this identity is larger than 95%. A polypeptide covered by this invention should be defined as comprising at least 80% identity over its entire length to a polypeptide sequences described herein. More preferably, this identity is larger than 90%, and even more preferably, the identity is larger than 95%.

The methods therefore included in this invention cover the use of a DNA or RNA molecule comprising an expression system, wherein said expression system is capable of producing a polynucleotide or polypeptide encoded therefrom when said expression system is present in a compatible host cell. This host cell may be a eukaryotic or bacterial host cell, and it may be used for a process for producing a polynucleotide or polypeptide by transforming or transfecting it with an expression system such that the host cell, under appropriate culture conditions, produces the encoded polynucleotide or polypeptide.

This invention also covers methods for the identification and development of compounds, agonist or antagonists, which are capable of interfering with the expression or function of a polynucleotide or polypeptide described herein. Such methods may include the following steps:
(a) contacting a candidate compound with cells which express a polypeptide, or cell membranes expressing said polypeptide, or respond to said polypeptide; and
(b) observing the binding, or stimulation or inhibition of a functional response, or comparing the ability of the cells or cell membranes which were contacted with the candidate compound with the same cells or cell membranes which were not contacted with said polypeptide; or
(c) observing the cellular localization of the polypeptide after contacting it with the candidate compound with the cellular localization of the polypeptide without contacting it to the candidate compound; or
(d) contacting a candidate compound with a polypeptide and observe the activity or structural condition of a polypeptide and comparing it to the activity or structural condition of a polypeptide which is not contacted with the candidate compound.

Also the following steps may be used for the identification of such compounds:
(a) contacting a candidate compound with cells which express said polynucleotide, or respond to said polynucleotide; and
(b) observing the stimulation or inhibition of a functional response, or comparing the ability of the cells which were contacted with the candidate compound with the same cells which were not contacted with said polynucleotide.

The diagnostic and therapeutic methods of this invention may be useful for diseases selected from the group of estrogen receptor-dependent breast cancer, estrogen receptor-independent breast cancer, hormone receptor-dependent prostate cancer, hormone receptor-independent prostate cancer, brain cancer, renal cancer, colon cancer, colorectal cancer, pancreatic cancer, bladder cancer, esophageal cancer, stomach cancer, genitourinary cancer, gastrointestinal cancer, uterine cancer, ovarian cancer, astrocytomas, gliomas, skin cancer, squamous cell carcinoma, Keratoakantoma, Bowen disease, cutaneous T-Cell Lymphoma, melanoma, basal cell carcinoma, actinic keratosis, sarcomas, Kaposi's sarcoma, osteosarcoma, head and neck cancer, small cell lung carcinoma, non-small cell lung carcinoma, leukemias, lymphomas, or other blood cell cancers, ichtiosis, acne, acne vulgaris, thyroid resistance syndrome, diabetes, thalassemia, cirrhosis, protozoal infection, rheumatoid arthritis, rheumatoid spondylitis, all forms of rheumatism, osteoarthritis, gouty arthritis, multiple sclerosis, insulin dependent diabetes mellitus, non-insulin dependent diabetes, asthma, rhinitis, uveithis, lupus erythematoidis, ulcerative colitis, Morbus Crohn, inflammatory bowel disease, chronic diarrhea, psoriasis, atopic dermatitis, bone disease, fibroproliferative disorders, atherosclerosis, aplastic anemia, DiGeorge syndrome, Graves' disease, epilepsia, status epilepticus, alzheimer's disease, depression, schizophrenia, schizoaffective disorder, mania, stroke, mood-incongruent psychotic symptoms, bipolar disorder, affective disorders, meningitis, muscular dystrophy, multiple sclerosis, agitation, cardiac hypertrophy, heart failure, reperfusion injury and/or obesity.

### DETAILED DESCRIPTION OF THE INVENTION

The following examples further describe the invention:

### EXAMPLE 1

SEQ ID NO:1 (A8)

One rat cDNA clone, originally derived from the above described SSH analysis of the mammary tumor test system was used to establish the corresponding EST (Expressed Sequence Tag) cluster from rat EST databases. The nucleotide sequence identity within the cluster was over 96%. The consensus sequence of this cluster was used to run a blast (Basic Local Alignment Search Tool, http://www.ncbi.nlm.nih.gov/BLAST/) analysis against mouse gene sequence databases. A sequence identity of 89% was found with the mouse mRNA BC005755, which again showed a 89% identity on the nucleotide sequence level to the mRNAs of the human MEP50 gene sequence. The corresponding NCBI (National Center for Biotechnology Information) reference sequence (http://www.ncbi.nlm.nih.gov/RefSeq/) for this locus, NM_{—}024102 has a length of 2428 nucleotides and codes for a protein of 342 amino acids. The gene MEP50 maps on chromosome 1.

MEP50 contains a G-protein beta WD-40 repeat according to a search with the database Pfam (Protein family alignment multiple). Pfam is a large collection of protein multiple sequence alignments and profile hidden Markov models (Bateman, 2000, Nucleic Acids Res. 30, 276-280).

MEP50 also contains a Glycosyl hydrolases family 18 motif. MEP50 was shown to be part of the Methylosome (Friesen, 2002, J. Biol. Chem. 277, 8243-8247) that is involved in the assembly of snRNP. Interestingly MEP50 was also shown to interact with the phosphatase FCP1, the only Pol II Phosphatase isolated so far (Licciardo, 2003, Nucleic Acids Res. 31, 999-1005).

In FIG. 1, a summary of established data for SEQ ID NO:1 is presented.

This sequence was shown to be differentially expressed in analysis of “In situ hybridization” (ISH) of matched human tumors (BioCat BA3, http://www.biocat.de), namely in cancers of the colon, stomach and breast, as exemplified in FIG. 3. Herein, data of ISH (In Situ Hybridization) experiments with Digoxygenin labelled RNA probes from the MEP50 locus (SEQ ID NO:1) are presented. RNA probes were generated with the DIG RNA labelling Kit from Roche according to the manufacturers instructions using a pOTB7 vector containing MEP50 (SEQ ID NO:1) sequences. Parraffin embedded tissue sections were deparaffinized, and postfixed in 4% paraformaldehyde. After incubation with proteinase K and washing, probes were denatured and hybridized to the slides at 65° over night. After several washes, the slides were subjected to a colorimetric assay using anti-digoxygenin antibodies (BM purple, Roche). Counterstain was done with H&E.

Tumor specific expression was further analyzed by hybridization experiments with Cancer Profiling Arrays (CA) from Clontech (http://www.bdbiosciences.com). The Cancer Profiling Arrays include normalized amplified cDNA from 241 tumor and corresponding normal tissues from individual patients, along with negative and positive controls, and cDNA from nine cancer cell lines. Here, overexpression was defined as upregulation of expression in the tumor probe versus expression in the normal probe of at least 1.5 fold. Percentage of upregulation in the tissues analysed is shown in FIG. 4. Herein, the cancer profiling expression analysis (CA) for SEQ ID NO:1 (MEP50) is presented. For this purpose, nylon filters carrying linear amplified cDNA from 241 tumor and corresponding normal tissues from individual patients (cancer filter arrays by Clontech) was hybridized with a radioactive labelled MEP50 (SEQ ID NO:1) cDNA. The signal of the tumor tissue was quantified by the phosphoimager analysis software AIDA (Fuji) and compared to the signal obtained by using corresponding hybridisation material of the normal tissue. The number of probe pairs per tissue is given in brackets. Definitions: A less than 0.7 fold expression of the sequence in the tumor sample is indicated as “DOWN”, whereas “Up” means an at least more than 1.5 fold expression of the sequence in the tumor sample, each time compared to the expression in normal tissue samples. Percentages of Up and Down-regulations are shown in the columns. Numbers of tumor samples analysed are indicated in brackets next to the tumor tissue origin analysed (bottom). MEP50 shows significant upregulation (in more than 50% of analyzed pairs) in tissue samples derived from cancers of the breast, uterus, colon, rectum and lung.

In FIG. 5, summary data for the cancer profiling expression analysis (CA) for SEQ ID NO:1-9 are presented according to the individual tumor tissue origin examined.

In order to functionally examine whether MEP50 could be causally involved in the process of tumor progression, MEP50 was transiently overexpressed or transiently downregulated by RNA interference in HEK-293T cells and subsequently potential resulting influences on tumor cell properties were assayed. Experiments shown in FIGS. 6 and 7 demonstrate that overexpression of MEP50 leads to increased proliferation, its downregulation to decreased proliferation. These findings are further supported by analysis of HT29 colon carcinoma cells and T47D mammary carcinoma cells stably overexpressing MEP50. As shown in FIG. 8, MEP50 increases proliferation in both cell types. Thus, MEP50 is causally involved in regulating the proliferation capacity of tumor cells. MEP50 also affects the invasion potential of tumor cells. As shown in FIG. 9, HT29 colon carcinoma cells stably overexpressing MEP50 have a stronger capacity to invade into Matrigel (BD biosciences) which represents the basement membrane matrix.

In respect to these functional analysis, in detail the following tests have been performed:

FIG. 6: Data from proliferation assays with transiently transfected HEK-293T cells.

A: For these tests, MEP50 and Ras cDNAs were cloned into the mammalian expression vector pCDNA3.1 (Invitrogen). HEK-293T cells were then transfected with expression vectors for the indicated proteins using Lipofectamine (Invitrogen) according to the manufacturers instructions. 16 h after transfection cells were seeded with 10,000 per well in triplicates in 96 well plates. From this time point on viable cells were determined every 24 h using the CellTiter Kit (Promega). The graphs represent the mean values of relative growth rates of three independent experiments. Note the increased growth rate upon expression of the Ras or MEP50 gene sequences.

B: Western Blot analysis testing the expression of the expressed proteins Ras and MEP50. For this purpose cells were lysed 24 h after transfection and lysates were subjected to gel electrophoresis and subsequent Western blotting with an anti-HA-antibody (12-CA-5). Note the clear expression of the proteins upon transfection of the expression constructs.

FIG. 7: Proliferation assay using siRNA treated HEK-293T cells.

A: Analysis of the efficiency of the interference with the target protein expression, here tested on the protein level. HEK-293T cells were transiently transfected with an expression vector for MEP50 and the indicated siRNAs. 48 h after transfection cells were lysed and lysates were subjected to gelelectrophoresis and subsequent Western Blotting with an anti-HA-antibody (12-CA-5). Note that the expression of the target protein MEP50 could be strongly inhibited by using the siRNA targeting the MEP50 gene transcripts.

B: HEK-293T were transfected with the indicated siRNAs using Lipofectamine (Invitrogen) according to the manufacturers instructions. 16 h after transfection cells were seeded with 10,000 cells per well in triplicates in 96 well plates. From this time point on viable cells were determined every 24 h using the non radioactive cell proliferation assay “Cell Titer 96” (Promega). The CellTiter 96 Assay is colorimetric method for determining the number of viable cells. It is composed of solutions of a novel tetrazolium compound [3-(4,5-dimethylthiazol-2-yl)-5-(3-carboxymethoxyphenyl)-2-(4-sulfophenyl)-2H]-tetrazolium, inner salt; MTS. MTS is bioreduced by cells into a formazan product that is soluble in tissue culture medium. The conversion of MTS into the aqueous soluble formazan product is accomplished by dehydrogenase enzymes found in metabolically active cells. The quantity of formazan product as measured by the amount of 490 nm absorbance is directly proportional to the number of living cells in culture. The graphs represent mean values for absorbance at 490 nm of three independent experiments. Note the inhibition of proliferation upon down-regulation of MEP50 expression using MEP50 specific siRNA molecules.

FIG. 8: Proliferation assays using overexpression studies.

HT29 colon cancer cells and T47D breast cancer cells were stably transfected with either control vector pCDNA3.1 or a corresponding expression vector derived thereof for MEP50. Stable mass cultures were selected using Neomycin. Cells were seeded with 10,000 cells per well in triplicates in 96 well plates. From this time point on viable cells were determined every 24 h using the CellTiter Kit (Promega). The graphs represent mean values for absorbance at 490 nm of three independent experiments. Note that the growth rate of both cell types is increased upon expression of MEP50.

FIG. 9: Invasion assay with stably transfected HT29 colon cancer cells. 10,0000 cells were seeded onto 2 mg/ml Matrigel in the upper compartment of a transwell migration chamber (8 μm pores). The lower compartment contained medium with 10% serum. After 48 or 72 h cell density on the lower surface of the membrane was determined by staining with crystal violett and measuring the OD at 595 nm as a measurement of invasion through the Matrigel structure. Note that upon expression of the MEP50 gene the cells display an increased invasive character.

In summary, MEP50 shows upregulation in metastasizing tumor cells versus non metastasizing tumor cells, and also displays upregulated expression in various tumor tissues versus normal tissue samples. Moreover, MEP50 is functionally involved in processes involved in tumor progression like increased proliferation and invasion. Therefore, this sequence may particularly be useful for staging of human tumor diseases, as well as for decisions on prognosis and treatment modalities. Furthermore, the MEP50 gene and its gene products may be used as target structures to develop therapeutic anti-cancer drugs.

SEQ ID NO: 1 (NM_024102)
cgtccagtttgagtctaggttggagttggaaccgtggagatgcggaaggaaaccccaccccccctagtgcccccggcggc
ccgggagtggaatcttcccccaaatgcgcccgcctgcatggaacggcagttggaggctgcgcggtaccggtccgatgggg
cgcttctcctcggggcctccagcctgagtgggcgctgctgggccggctccctctggctttttaaggacccctgtgccgcc
cccaacgaaggcttctgctccgccggagtccaaacggaggctggagtggctgacctcacttgggttggggagagaggtat
tctagtggcctccgattcaggtgctgttgaattgtgggaactagatgagaatgagacacttattgtcagcaagttctgca
agtatgagcatgatgacattgtgtctacagtcagtgtcttgagctctggcacacaagctgtcagtggtagcaaagacatc
tgcatcaaggtttgggaccttgctcagcaggtggtactgagttcataccgagctcatgctgctcaggtcacttgtgttgc
tgcctctcctcacaaggactctgtgtttctttcatgcagcgaggacaatagaattttactctgggatacccgctgtccca
agccagcatcacagattggctgcagtgcgcctggctaccttcctacctcgctggcttggcatcctcagcaaagtgaagtc
tttgtctttggtgatgagaatgggacagtctcccttgtggacaccaagagtacaagctgtgtcctgagctcagctgtaca
ctcccagtgtgtcactgggctggtgttctccccacacagtgttcccttcctggcctctctcagtgaagactgctcacttg
ctgtgctggactcaagcctttctgagttgtttagaagccaagcccacagagactttgtgagagatgcgacttggtccccg
ctcaatcactccctgcttaccacagtgggctgggaccatcaggtcgtccaccacgttgtgcccacagaacctctcccagc
ccctggacctgcaagtgttactgagtagattggatttaagacaaaaagcaagtcccccatgagtgtccacttctttgccc
tgccctctcagcttgtgagacaacacaggagccttctatagtatgttgatatgctagatctgtgccgttaataggcatcg
tctctcagcctgagggaggctggattctgggttcctgtagtcacagggaggaaaagctttcttaaaaatggacatgtatg
tgcgtgtgagtgtgtgtgtagatttatagtttttggtagtggcaggaataaaaaaaatccatcctacatcttccctaagc
actgcctctctctcaccccccaaaacaagttgacgaaagggttttatgtagctgtctatgaggaattggccgtgtctggg
tgggttatgggatgtgggcatccctgggttcttggaagcagctcttatgctactcatagagatgggattgactttatttt
tttatagtgcttaattcaccattatgagaaatgcttccagtcacaaaaatgcagcccagctcactctgaggaagaagcag
gacttggtacggttttacacaactccttaccattaaactgaatcagaaatccattttctggctgaataaaaagtttggct
tgcctgtgtaatgcccactcccttccccctggctccctagtgatgggacatatatgagagagaagtgtttttctatcata
gacaccataggggaaagtttggggatgaaggagagcttaaaggtgtttcaattaagttagaaaactgacacaggctgttg
agaattctttgccacttttcccaccccaaaacagcatggggcctgacatcttctgccctggtcccctttctcttgatgtg
gaaagtctgaatgcagtatttatagacttctaaggttttaaaatccagtatcaagaagaaaatcagaaatactggttggt
gaaataaagagtttaggcattgttggcctgtcttttttgaagcatgtgtgttatgtgtagttagatatatttcacttatg
tgagtcatcatggtgttggtcttgtagcccattatttttcctgtgcttccccagcttcccaaagtagctagttagaactt
aaggtaaatatttattcttgggttggtggagtggatattgccagttaggagtcatggatcaattactgattatattgaaa
gtaaatataatcaattatgtacttttgagctttgcaggttcaatttaggtaaaaatcacattatgaaactgggaaagtct
gaaggaatatgggcaaaatatttctcagtaaagcttccatgcttcacccttgacatgattacccttgagtaaaacatggg
aatttgtaaaaaaaaaaaaaaaaaaaaa
SEQ ID NO: 10 - PROTEIN (NP_077007)
MRKETPPPLVPPAAREWNLPPNAPACMERQLEAARYRSDGALLLGASSLSGRCWAGSLWLFKDPCAAPNEGFCSAGVQTEAG
VADLTWVGERGILVASDSGAVELWELDENETLIVSKFCKYEHDDIVSTVSVLSSGTQAVSGSKDICIKVWDLAQQVVLSSYR
AHAAQVTCVAASPHKDSVFLSCSEDNRILLWDTRCPKPASQIGCSAPGYLPTSLAWHPQQSEVFVFGDENGTVSLVDTKSTS
CVLSSAVHSQCVTGLVFSPHSVPFLASLSEDCSLAVLDSSLSELFRSQAHRDFVRDATWSPLNHSLLTTVGWDHQVVHHVVP
TEPLPAPGPASVTE

The combined data established for SEQ ID NO:1 together with the data for SEQ ID NO:2-9 and selected additional sequences are presented in summary in FIG. 1 which comprises a list of the cancer antigens identified, characterized and presented in this invention. Here, the identities of the cancer antigens of SEQ ID NO:1-9 are especially indicated.

Names and/or accession numbers (Acc. No.) of differentially expressed sequences are given. According to data derived from Microarray Analysis (gene expression analysis), in total 89 sequences were found to be differentially expressed in at least one pair of metastasizing versus non metastasizing cells (indicated as a “+” mark in the column Microarray). These Microarray Analysis experiments were performed as described in FIG. 2. Some of the sequences listed in the table have been shown to be differentially expressed (indicated as a “+” mark in the column ISH) also by performing “In situ Hybridization” (ISH) experiments with matched human normal and tumor tissue samples derived from at least three tissue types.

Several sequences were also analyzed in in Cancer profiling Arrays (CA): Here, overexpression of a given gene (indicated as a “+” mark in the column CA) was defined as upregulation of expression in the tumor probe versus the normal probe in at least 50% of analyzed pairs which were derived from at least 3 of 8 different tissues analyzed.

In addition, FIG. 1 also contains information on indications for functional involvement of the single sequences in metastatic processes. A positive “+” mark in this context indicates that a given cancer antigen gave rise to an at least 20% change of activity over control in at least one functional assay. For detailed information on functional assays see FIG. 6-9.

Nine sequences were estimated as positive (“+” mark in the column functional indications) for at least three out of four criteria measured for having a relevance in metastatic processes (i.e. measurements of the following tests: Analyses in Microarray, ISH, CA, functional tests). These sequences are highlighted and refer to SEQ ID NO:1-9. Detailed descriptions of these SEQ ID NO:1-9 are given in Examples 1-9. The column “ID” lists the internal identification number, “Sequence No” gives the number of the sequence used in the text.

In FIG. 2, raw Microarray analysis data from hybridization tests with cDNA from the endometrial cancer cell line HEC-1A versus the metastasizing endometrial cancer cell line AN3-CA (ATCC HTB-112 and -111) are presented, including in exemplified manner the analysis of the expression of SEQ ID NO:1, which is annotated as sequence A8 in FIG. 2. Diagnostic tools in the form of cDNA chips were made by spotting 4 ng of each cDNA for the 89 genes listed in FIG. 1 onto glass slides. Each gene was spotted 6 times in duplets. In addition, 4 housekeeping genes were spotted (HPRT, β-Actin, α-Tubulin, Ubiquitin). For hybridisation purposes, 1.5 μg poly A⁺ RNA isolated from the cell lines listed in example 10 was reverse transcribed and labelled using the Cyscribe Kit (Amersham). In one half of the experiment RNA from the non metastasizing cells was labelled with Cy3, and RNA from the metastasizing cells with Cy5 (left side FIG. 2A). In the other half of the experiment RNA from the non metastasizing cells was labelled with Cy5, and RNA from the metastasizing cells with Cy3 (right side FIG. 2A). Probes were mixed and hybridized to the cDNA chips. Representative sections of the cDNA chips are shown in A. Gene sequences (cancer antigens) upregulated in the metastasizing cells light up red on the left side, and light up green on the right side. Yellow spots indicate unchanged expression. B: The spotting scheme for the sections of the cDNA chips shown in A is presented. C: Regulation factors for the expression of the five genes shown in A are given. Averages from 12 spots of the 635/532 nm signal in the column Cy3/Cy5, and of the 532/635 nm signal in the column Cy5/Cy3 are shown. “Mean” is the average of the Cy3/Cy5 and the Cy5/Cy3 value. Note: A regulation factor of, e.g., 5.01 as estimated as Mean value for the sequence annotated as A8, which represents SEQ ID NO:1, refers to a 5.01 fold overexpression of this sequence in the metastasising cells in comparison to the non metastasising tumor cells.

### EXAMPLE 2

SEQ ID NO:2 (E4)

Another rat cDNA clone, originally derived from the above described SSH analysis of the pancreatic tumor test system was used to establish the corresponding EST cluster from rat EST databases. Nucleotide sequence identity with an identified rat sequence cluster was over 96%. Three further clones derived from this pancreatic test system also matched to this gene sequence cluster with over 96% nucleotide sequence identity. The consensus sequence of this cluster was established by using the software DNAStar, SeqManII (http://www.dnastar.com/), and was subsequently used in blast analysis using the human genome sequence database BLAT (http://genome.ucsc.edu/cgi-bin/hgBlat?command=start). This way, a nucleotide sequence identity of 90% was identified with the human mRNA AK130372 representing the locus FAM49B (family with sequence similarity 49, member B), alias BM-009. The corresponding NCBI reference sequence for this locus, NM_{—}016623 comprises a length of 2219 nucleotides and codes for a predicted protein of unknown function. According to the AceView application, different transcripts of this gene exist, altogether putatively encoding 19 different protein isoforms.

AceView represents an integrated view of the human genes as reconstructed by alignment of all publicly available mRNAs and ESTs on the genome sequence (http://www.ncbi.nih.gov/IEB/Research/Acembly/index.html?human).

The amino acid sequence of FAM49B was analyzed by PSORT, a computer program for the prediction of protein localization sites in cells. According to PSORT2 (http://psort.nibb.ac.jp) the proteins encoded by this RNA are most likely located in the cytoplasm. The amino acid sequence of FAM49B was also analyzed by Pfam search. According to this analysis this protein belongs to a family of several hypothetical eukaryotic proteins (DUF1394) of around 320 residues in length. The functions of this protein family are unknown. The gene is localized in the 8q24 region, an area found to be minimally overepresented in prostate cancer (Tsuchiya, 2000, Am. J. Pathol. 160, 1799-1806).

In FIG. 1, a summary of established data for SEQ ID NO:2 is presented.

This sequence was shown to be differentially expressed in Microarray Analysis comparing samples of metastasizing versus non metastasizing cells as exemplified for SEQ ID NO:1 in FIG. 2. Tumor specific expression was further analyzed by hybridization experiments with Cancer Profiling Arrays (CA) from Clontech (http://www.bdbiosciences.com). The estimated percentages of upregulation in the tissues analyzed is shown in FIG. 5. FAM49B shows significant upregulation (in more than 50% of analyzed pairs) in uterus, ovary, colon and rectum.

In order to functionally examine whether FAM49B could be causally involved in the process of tumor progression, it was transiently overexpressed or transiently downregulated by RNA interference in HEK-293T cells and subsequently potential resulting influences on tumor cell properties were assayed. For overexpression a sequence corresponding to the NCBI reference sequence (http://www.ncbi.nlm.nih.gov/RefSeq/) was used. Experiments as previously exemplified for SEQ ID NO:1 in FIGS. 6-8 demonstrate, that overexpression of FAM49B leads to increased proliferation, whereas its downregulation results in decreased proliferation.

Furthermore, FAM49B also affects the invasion potential of tumor cells.

In summary, FAM49B shows upregulation in metastasizing tumor cells versus non metastasizing tumor cells, and also displays upregulated expression in various tumor tissues versus normal tissue samples. Moreover, FAM49B is functionally involved in processes involved in tumor progression like increased proliferation and invasion. Therefore, this sequence may particularly be useful for staging of human tumor diseases, as well as for decisions on prognosis and treatment modalities. Furthermore, the FAM49B gene and its gene products may be used as target structures to develop therapeutic anti-cancer drugs.

SEQ ID NO: 2 (NM_016623)
ggcaggtgttgaggggctcccggtccggctgccgccgctcccccgctccggacccggggctccccctagcgccgctgagg
agccgcctctgcggctccaggagggcgcaggagcgggactgagagcgcctggaggctcgagcggagggtaattcatttgc
acacctgttagcaagaaacagaagttgaaggactggaacaagtgaactaggaaagagggaacgccaatccaaggatagaa
ggacaaggacagaatcaccagcactggctgaaggcctcctgtttcctgcgctttctccttttcctgtgaaatctccgagg
agaagaaagaatgatggacagtttatcctttcactgccacaaggcctgtttacttggcagtaggtccttaagttccttgc
ttttttgctgctgtttggtgactggaagaggcaccagagactctcactctggggaggtttgctggcatgggtaatctcat
taaggtgctaaccagggacatagaccacaatgcagcacattttttcttggactttgaaagtaccttaacatggggaatct
tcttaaagttttgacatgcacagaccttgagcaggggccaaattttttccttgattttgaaaatgcccagcctacagagt
ctgagaaggaaatttataatcaggtgaatgtagtattaaaagatgcagaaggcatcttggaggacttgcagtcatacaga
ggagctggccacgaaatacgagaggcaatccagcatccagcagatgagaagttgcaagagaaggcatggggtgcagttgt
tccactagtaggcaaattaaagaaattttacgaattttctcagaggttagaagcagcattaagaggtcttctgggagcct
taacaagtaccccatattctcccacccagcatctagagcgagagcaggctcttgctaaacagtttgcagaaattcttcat
ttcacactccggtttgatgaactcaagatgacaaatcctgccatacagaatgatttcagctattatagaagaacattgag
tcgtatgaggattaacaatgtaccggcagaaggagaaaatgaagtaaataatgaattggcaaatcgaatgtctttgtttt
atgctgaggcaactccaatgctgaaaaccttgagtgatgccacaacaaaatttgtatcagagaataaaaatttaccaata
gaaaataccacagattgtttaagcacaatggctagtgtatgcagagtcatgctggaaacaccggaatacagaagcagatt
tacaaatgaagagacagtgtcattctgcttgagggtaatggtgggtgtcataatactctatgaccacgtacatccagtgg
gagcatttgctaaaacttccaaaattgatatgaaaggttgtatcaaagttcttaaggaccaacctcctaatagtgtggaa
ggtcttctaaatgctctcaggtacacaacaaaacatttgaatgatgagactacctccaagcaaattaaatccatgctgca
ataacaattctggaataagcacctgctgtagacagaagacagtattctgcaatgactgagaatgcagttttttagtgatt
gcaattactatctcatttattcttgcttttatttctttcctctgttcctcttccctcttttttaatcatgttcttaagac
ttcttttctgtgccaaaatcagtaaagttacactctgaagggatatcatcctttcaaacgggccatctaaggcagctaat
tatgcattgcattggggtctctactgagaaaaattctgtgacttgaactaaatatttttaaatgtggattttttttgaaa
ctaatatttaatattgcttctcctgcatggcaaaactgcctattctgctatttaaaaaccctcaatgactttattttcta
ctgccgcctttttcatgtgcaaccaaaatgaaaatgtttaaattaactgtgttgtacaaatggtacccaacacaaacttt
ttttaaattagtaatacttttgtttaaagttttaagtttgcattttgactttttttgtaaggatgtatgttgtgtgttta
acctttattaactaacgttaaaagctgtgatgtgtgcgtagaatattacgtatgcatgttcatgtctaaagaatggctgt
tgatgataaaataaaaatcagctttcatttttctaaaaaaaaaaaaaaaaaaaaaaaaa
SEQ ID NO: 11 - PROTEIN (NP_057707)
MGNLLKVLTCTDLEQGPNFFLDFENAQPTESEKEIYNQVNVVLKDAEGILEDLQSYRGAGHEIREAIQHPADEKLQEKAWGA
VVPLVGKLKKFYEFSQRLEAALRGLLGALTSTPYSPTQHLEREQALAKQFAEILHFTLRFDELKMTNPAIQNDFSYYRRTLS
RMRINNVPAEGENEVNNELANRMSLFYAEATPMLKTLSDATTKFVSENKNLPIENTTDCLSTMASVCRVMLETPEYRSRFTN
EETVSFCLRVMVGVIILYDHVHPVGAFAKTSKIDMKGCIKVLKDQPPNSVEGLLNALRYTTKHLNDETTSKQIKSMLQ

### EXAMPLE 3

SEQ ID NO:3 (H3)

Another rat cDNA clone, originally derived from the above described SSH analysis of the pancreas tumor test system was used to establish the corresponding EST cluster from rat EST databases. Identity to the ESTs within this cluster was 98%. Identity within the cluster was over 96%. The consensus sequence of this cluster was used to blast against human genome sequence databases. An identity of 89% was found to the human mRNA NM_{—}024085 representing the locus FLJ22169. The reference RNA has a length of 3816 nucleotides and codes for a predicted protein of unknown function with 839 amino acids. According to Pfam Search the predicted protein shares homology to Autophagy protein Apg9. In yeast, 15 Apg proteins coordinate the formation of autophagosomes. Autophagy is a bulk degradation process induced by starvation in eukaryotic cells. Apg9 plays a direct role in the formation of the cytoplasm to vacuole targeting and autophagic vesicles, possibly serving as a marker for a specialised compartment essential for these vesicle-mediated alternative targeting pathways. According to Psort2, this protein most likely localizes to the membrane. According to AceView, this gene produces, by alternative splicing, 9 different transcripts altogether encoding 9 different protein isoforms.

In FIG. 1, a summary of established data for SEQ ID NO:3 is presented.

This sequence was shown to be differentially expressed in Microarray Analysis comparing samples of metastasizing versus non metastasizing tumor cells as exemplified for SEQ ID NO:1 in FIG. 2. Tumor specific expression was further analyzed in hybridization experiments using Cancer Profiling Arrays (CA) from Clontech (http://www.bdbiosciences.com). Estimations of percentages of upregulation in the tissues analyzed is shown in FIG. 5. FLJ22169 shows significant upregulation of expression (in more than 50% of analyzed pairs) in tissues derived from cancers of the uterus, ovary, colon and rectum.

In order to functionally examine whether FLJ22169 could be causally involved in the progression of tumor progression, it was transiently overexpressed or transiently downregulated by RNA interference in HEK-293T cells and subsequently potential resulting influences on tumor cell properties were assayed. For its overexpression a sequence corresponding to the NCBI reference sequence (http://www.ncbi.nlm.nih.gov/RefSeq/) was used. Experiments as previously exemplified for SEQ ID NO:1 in FIGS. 6 and 7, demonstrate that also overexpression of FLJ22169 leads to increased proliferation, its downregulation results in decreased proliferation. FLJ22169 also affects invasion potential of tumor cells in experiments performed according to those exemplified for SEQ ID NO:1 in FIG. 9.

In summary, FLJ22169 shows upregulation in metastasizing tumor cells versus non metastasizing tumor cells, and also displays upregulated expression in various tumor tissues versus normal tissue samples. Moreover, FLJ22169 is functionally involved in processes involved in tumor progression like increased proliferation and invasion. Therefore, this sequence may particularly be useful for staging of human tumor diseases, as well as for decisions on prognosis and treatment modalities. Furthermore, the FLJ22169 gene and its gene products may be used as target structures to develop therapeutic anti-cancer drugs.

SEQ ID NO: 3 (NM_024085)
ggggtcgcgccgagccgagccgagccgagcggagccggcggagcctctggaatcacccgggtcgctgttcctgaggtggt
caaggtggacagggggcggtggtgatggcgcagtttgacactgaataccagcgcctagaggcctcctatagtgattcacc
cccaggggaggaggacctgttggtgcacgtcgccgaggggagcaagtcaccttggcaccgtattgaaaaccttgacctct
tcttctctcgagtttataatctgcaccagaagaatggcttcacatgtatgctcatcggggagatctttgagctcatgcag
ttcctctttgtggttgccttcactaccttcctggtcagctgcgtggactatgacatcctatttgccaacaagatggtgaa
ccacagtcttcaccctactgaacccgtcaaggtcactctgccagacgcctttttgcctgctcaagtctgtagtgccagga
ttcaggaaaatggctcccttatcaccatcctggtcattgctggtgtcttctggatccaccggcttatcaagttcatctat
aacatttgctgctactgggagatccactccttctacctgcacgctctgcgcatccctatgtctgcccttccgtattgcac
gtggcaagaagtgcaggcccggatcgtgcagacgcagaaggagcaccagatctgcatccacaaacgtgagctgacagaac
tggacatctaccaccgcatcctccgtttccagaactacatggtggcactggttaacaaatccctcctgcctctgcgcttc
cgcctgcctggcctcggggaagctgtcttcttcacccgtggtctcaagtacaactttgagctgatcctcttctggggacc
tggctctctgtttctcaatgaatggagcctcaaggccgagtacaaacgtggggggcaacggctagagctggcccagcgcc
tcagcaaccgcatcctgtggattggcatcgctaacttcctgccgtgccccctcatcctcatatggcaaatcctctatgcc
ttcttcagctatgctgaggtgctgaagcgggagccgggggccctgggagcacgctgctggtcactctatggccgctgcta
cctccgccacttcaacgagctggagcacgagctgcagtcccgcctcaaccgtggctacaagcccgcctccaagtacatga
attgcttcttgtcacctcttttgacactgctggccaagaatggagccttcttcgctggctccatcctggctgtgcttatt
gccctcaccatttatgacgaagatgtgttggctgtggaacatgtgctgaccaccgtcacactcctgggggtcaccgtgac
cgtgtgcaggtcctttatcccggaccagcacatggtgttctgccctgagcagctgctccgcgtgatcctcgctcacatcc
actacatgcctgaccactggcagggtaatgcccaccgctcgcagacccgggacgagtttgcccagctcttccagtacaag
gcagtgttcattttggaagagttgttgagccccattgtcacacccctcatcctcatcttctgcctgcgcccacgggccct
ggagattatagacttcttccgaaacttcaccgtggaggtcgttggtgtgggagatacctgctcctttgctcagatggatg
ttcgccagcatggtcatccccagtggctatctgctgggcagacagaggcctcagtgtaccagcaagctgaggatggaaag
acagagttgtcactcatgcactttgccatcaccaaccctggctggcagccaccacgtgagagcacagccttcctaggctt
cctcaaggagcaggttcagcgggatggagcagctgctagcctcgcccaagggggtctgctccctgaaaatgccctcttta
cgtctatccagtccttacaatctgagtctgagcccctgagccttatcgcaaatgtggtagctggctcatcctgccggggc
cctccactgcccagagacctgcagggctccaggcacagggctgaagtcgcctctgccctgcgctccttctccccgctgca
acccgggcaggcgcccacaggccgggctcacagcaccatgacaggctctggggtggatgccaggacagccagctccggga
gcagcgtgtgggaaggacagctgcagagcctggtgctgtcagaatatgcatccacagagatgagcctgcatgccctctat
atgcaccagctccacaagcagcaggcccaggctgaacctgagcggcatgtatggcaccgccgggagagtgatgagagtgg
agaaagcgcccctgatgaagggggagagggcgcccgggccccccagtctatccctcgctctgctagctatccctgtgtag
caccccggcctggagctcctgagaccaccgccctgcatgggggcttccagaggcgctacggtggcatcacagatcctggc
acagtgcccagggttccctctcatttctctcggctgcctcttggagggtgggcagaagatgggcagtcggcatcaaggca
ccctgagcccgtgcccgaagagggctcggaggatgagctaccccctcaggtgcacaaggtatagacaaggctgagcaggg
ttcctgtggcccaggatggaggccaccgctgccctgccatcccgtctgcctgccatgggacggctcctctgagtgttccc
tggccccatgtgtgtggtgtttgtgtgtctgtgcctggccaagggaggtgccaacactgggcttgccacagccccaggag
aggaatttggggcctaggaaccgagggcacacgggactctagcctcatccccaggacccccttggctcagagtgtggtgc
tagaaactggtccccagcccagccccagtactgccacctttacacctacccctgcaagtccccagagggctgcccacgat
agaagctgccaagcagggagaacctgtgccaactgtggagtggggaggttgggcctggaccctcaacccctgcaaccttc
cctagccccctcaatagatgagcaggtcaggctgtggcccttacctcacccgcagttctcgcccagtgctgcagccggct
cacctctctccgcttcttgcacatcactggcctgtgtgtgctgcttgctcctgttctgttcgcttgctcccgttccgttc
ggcttttgctttgcgttagggtgaagaccctagcgtccagctcccctcaacgctatattttgacactaaaaaagaaggtt
tctaaattgtaggagcaggatggaaatactttgctgcccttgccatcttttaggatgggcccccaggagactgaggtctt
cctgggccctcattgctgcttatcgtaccccccatcacctgcacatgggacagaccgggctggagggtgaccttggctgt
gtacgtcccagcaaaagagctctggcccgcatctcgctgtgccctgaagggggatgaagggcgatgcctcgcccgaggct
ttgggctgctgcactgcatgctgggactgctcctactctctgtcccacccctcacccagctgtggtccggctttgggaga
gtggtgaattgcgctgcccgaactcggagcggagcagggtagggaccgtgtacagcttgataacccttaataaaaaggga
gtttgaccagaaaaaaaaaaaaaaaaaaaaaaaaaaaaaaaaaaaaaaaaaaaaaaaaaaaaaaaaaaaaaaaaaagaaa
aaaaaaaaaaaaaaaagaaaaaaaaaaaaaaaaagaaaaaaaaaaaaaaaaaacct
SEQ ID NO: 12 - PROTEIN (NP_076990)
MAQFDTEYQRLEASYSDSPPGEEDLLVHVAEGSKSPWHRIENLDLFFSRVYNLHQKNGFTCMLIGEIFELMQFLFVVAFTTF
LVSCVDYDILFANKMVNHSLHPTEPVKVTLPDAFLPAQVCSARIQENGSLITILVIAGVFWIHRLIKFIYNICCYWEIHSFY
LHALRIPMSALPYCTWQEVQARIVQTQKEHQICIHKRELTELDIYHRILRFQNYMVALVNKSLLPLRFRLPGLGEAVFFTRG
LKYNFELILFWGPGSLFLNEWSLKAEYKRGGQRLELAQRLSNRILWIGIANFLPCPLILIWQILYAFFSYAEVLKREPGALG
ARCWSLYGRCYLRHFNELEHELQSRLNRGYKPASKYMNCFLSPLLTLLAKNGAFFAGSILAVLIALTIYDEDVLAVEHVLTT
VTLLGVTVTVCRSFIPDQHMVFCPEQLLRVILAHIHYMPDHWQGNAHRSQTRDEFAQLFQYKAVFILEELLSPIVTPLILIF
CLRPRALEIIDFFRNFTVEVVGVGDTCSFAQMDVRQHGHPQWLSAGQTEASVYQQAEDGKTELSLMHFAITNPGWQPPREST
AFLGFLKEQVQRDGAAASLAQGGLLPENALFTSIQSLQSESEPLSLIANVVAGSSCRGPPLPRDLQGSRHRAEVASALRSFS
PLQPGQAPTGRAHSTMTGSGVDARTASSGSSVWEGQLQSLVLSEYASTEMSLHALYMHQLHKQQAQAEPERHVWHRRESDES
GESAPDEGGEGARAPQSIPRSASYPCVAPRPGAPETTALHGGFQRRYGGITDPGTVPRVPSHFSRLPLGGWAEDGQSASRHP
EPVPEEGSEDELPPQVHKV

### EXAMPLE 4

SEQ ID NO:4 (B3)

Another rat cDNA clone, derived from the above described SSH analysis of the mammary tumor test system showed 99% identity to the rat mRNA CB717750. The corresponding rat EST cluster was used for a blast analysis against human genome databases. An identity of 90% was found on the nucleotide level to the human mRNA AK000178 representing the locus FLJ20171 which maps on chromosome 8. According to AceView, this locus produces, by alternative splicing, 13 different transcripts altogether encoding 13 different protein isoforms.

The corresponding NCBI Reference sequence NM_{—}017697 comprises 2140 nucleotides and encodes a hypothetical protein of 358 amino acids. According to SMART analysis (Simple Modular Architecture Research Tool, http://smart.embl-heidelberg.de/) this protein contains a RNA recognition motif known as the eukaryotic putative RNA-binding region RNP-1 signature or RNA recognition motif (RRM). RRMs are found in a variety of RNA binding proteins, including heterogeneous nuclear ribonucleoproteins (hnRNPs), proteins implicated in regulation of alternative splicing, and protein components of small nuclear ribonucleoproteins (snRNPs). The motif also appears in a few single stranded DNA binding proteins. The RRM structure consists of four strands and two helices arranged in an alpha/beta sandwich, with a third helix present during RNA binding in some cases.

In FIG. 1, a summary of established data for SEQ ID NO:4 is presented.

This sequence was shown to be differentially expressed in Microarray Analysis comparing samples of metastasizing versus non metastasizing tumor cells as previously exemplified for SEQ ID NO:1 in FIG. 2. Tumor specific expression was further analyzed in hybridization experiments using Cancer Profiling Arrays (CA) from Clontech (http://www.bdbiosciences.com). Estimations of percentages of upregulation in the tissues analyzed is shown in FIG. 5. FLJ20171 shows significant upregulation (in more than 50% of analyzed pairs) in tissues derived from cancers of the uterus, ovary and lung.

In order to functionally examine whether FLJ20171 could be causally involved in the progression of tumor progression, it was transiently overexpressed or transiently downregulated by RNA interference in HEK-293T cells and subsequently potential resulting influences on tumor cell properties were assayed. For its overexpression a sequence corresponding to the NCBI reference sequence (http://www.ncbi.nlm.nih.gov/RefSeq/) was used. Experiments as previously exemplified for SEQ ID NO:1 in FIGS. 6-8, demonstrate that also overexpression of FLJ20171 leads to increased proliferation, its downregulation results in decreased proliferation. FLJ20171 also affects invasion potential of tumor cells, as observed in experiments performed according to those exemplified for SEQ ID NO:1 in FIG. 9.

In summary, FLJ20171 shows upregulation in metastasizing tumor cells versus non metastasizing tumor cells, and also displays upregulated expression in various tumor tissues versus normal tissue samples. Moreover, FLJ20171 is functionally involved in processes involved in tumor progression like increased proliferation and invasion. Therefore, this sequence may particularly be useful for staging of human tumor diseases, as well as for decisions on prognosis and treatment modalities. Furthermore, the FLJ20171 gene and its gene products may be used as target structures to develop therapeutic anti-cancer drugs.

SEQ ID NO: 4 (NM_017697)
gaattcaagaaatgttgccctggttcacctgatattgacaaactggacgttgccacaatgacagagtatttaaattttga
gaagagtagttcagtctctcgatatggagcctctcaagttgaagatatggggaatataattttagcaatgatttcagagc
cttataatcacaggttttcagatccagagagagtgaattacaagtttgaaagtggaacttgcagcaagatggaacttatt
gatgataacaccgtagtcagggcacgaggtttaccatggcagtcttcagatcaagatattgcaagattcttcaaaggact
caatattgccaagggaggtgcagcactttgtctgaatgctcagggtcgaaggaacggagaagctctggttaggtttgtaa
gtgaggagcaccgagacctagcactacagaggcacaaacatcacatggggacccggtatattgaggtttacaaagcaaca
ggtgaagatttccttaaaattgctggtggtacttccaatgaggtagcccagtttctctccaaggaaaatcaagtcattgt
tcgcatgcgggggctccctttcacggccacagctgaagaagtggtggccttctttggacagcattgccctattactgggg
gaaaggaaggcatcctctttgtcacctacccagatggtaggccaacaggggacgcttttgtcctctttgcctgtgaggaa
tatgcacagaatgcgttgaggaagcataaagacttgttgggtaaaagatacattgaactcttcaggagcacagcagctga
agttcagcaggtgctgaatcgattctcctcggcccctctcattccacttccaacccctcccattattccagtactacctc
agcaatttgtgccccctacaaatgttagagactgtatacgccttcgaggtcttccctatgcagccacaattgaggacatc
ctggatttcctgggggagttcgccacagatattcgtactcatggggttcacatggttttgaatcaccagggccgcccatc
aggagatgcctttatccagatgaagtctgcggacagagcatttatggctgcacagaagtgtcataaaaaaaaacatgaag
gacagatatgttgaagtctttcagtgttcagctgaggagatgaactttgtgttaatggggggcactttaaatcgaaatgg
cttatccccaccgccatgtaagttaccatgcctgtctcctccctcctacacatttccagctcctgctgcagttattccta
cagaagctgccatttaccagccctctgtgattttgaatccacgagcactgcagccctccacagcgtactacccagcaggc
actcagctcttcatgaactacacagcgtactatcccagccccccaggttcgcctaatagtcttggctacttccctacagc
tgctaatcttagcggtgtccctccacagcctggcacggtggtcagaatgcagggcctggcctacaatactggagttaagg
aaattcttaacttcttccaaggttaccagtgtttgaaagatgtatggtgatcttgaaacctccagacacaagaaaacttc
tagcaaattcaggggaagtttgtctacactcaggctgcagtattttcagcaaacttgattggacaaacgggcctgtgcct
tatcttttggtggagtgaaaaagtttgagctagtgaagccaaatcgtaacttacagcaagcagcatgcagcatacctggc
tctttgctgattgcaaataggcatttaaaatgtgaatttggaatcagatgtctccattacttccagttaaagtggcatca
taggtgtttcctaagttttaagtcttggataaaaactccaccagtgtctaccatctccaccatgaactctgttaaggaag
cttcatttttgtatattcccgctcttttctcttcatttccctgtcttctgcataatcatgccttcttgctaagtaattca
agcataagatcttggaataataaaatcacaatcttaggagaaagaataaaattgttattttcccagtctcttggccatga
tgatatcttatgattaaaaacaaattaaattttaaaacacctgaaaaaaaaaaaaaaaaa
SEQ ID NO: 13 - PROTEIN (NP_060167)
MTEYLNFEKSSSVSRYGASQVEDMGNIILAMISEPYNHRFSDPERVNYKFESGTCSKMELIDDNTVVRARGLPWQSSDQDIA
RFFKGLNIAKGGAALCLNAQGRRNGEALVRFVSEEHRDLALQRHKHHMGTRYIEVYKATGEDFLKIAGGTSNEVAQFLSKEN
QVIVRMRGLPFTATAEEVVAFFGQHCPITGGKEGILFVTYPDGRPTGDAFVLFACEEYAQNALRKHKDLLGKRYIELFRSTA
AEVQQVLNRFSSAPLIPLPTPPIIPVLPQQFVPPTNVRDCIRLRGLPYAATIEDILDFLGEFATDIRTHGVHMVLNHQGRPS
GDAFIQMKSADRAFMAAQKCHKKKHEGQIC

### EXAMPLE 5

SEQ ID NO:5 (D2)

Another rat cDNA clone was used to establish the corresponding EST cluster from rat EST databases. Identity within the cluster was over 96%. The consensus sequence of this cluster was used for a blast analysis against human genome databases. An identity of 80% was found to the human mRNA NM_{—}030815 representing the locus C20orf126 which maps on chromosome 20. The Ensembl Genome Browser (http://www.ensembl.org/Homo_sapiens/) predicts that it produces one transcript with a length of 1290 bp. The coding sequence of the protein between the first in frame amino acid and the stop codon contains 176 residues. The first methionine corresponds to amino acid 44. The calculated molecular weight of the protein product is 15.5 kD.

Bioinformatic analysis according to PSORTII predicts that the subcellular localization of this protein is expected to be in the nucleus. Besides a nuclear localization signal, the predicted protein contains coiled coil domains. Such coiled coil structures (Psort Motiv, http://psort.nibb.ac.jp/) are found in some structural proteins, e.g. myosins, and in some DNA binding proteins as the so called leucine zipper. In this structure two α-helices bind each other forming a coil, in which this helices show a 3.5 residue periodicity which is slightly different from the typical value estimated at 3.6. Thus, the detection of coiled coil structure by searching for 7-residue periodicity is relatively more accurate than usual secondary structure prediction. Currently a classical detection algorithm developed by A. Lupas is used (Lupas, 1991, Science 252, 1162-1164). The function of C20orf126 is still unknown. Pfam analysis shows that this protein does not belong to any recognized protein family.

In FIG. 1, a summary of established data for SEQ ID NO:5 is presented.

This sequence was shown to be differentially expressed in Microarray Analysis comparing samples of metastasizing versus non metastasizing cells as previously exemplified for SEQ ID NO:1 in FIG. 2. Tumor specific expression was further analyzed in hybridization experiments using Cancer Profiling Arrays (CA) from Clontech (http://www.bdbiosciences.com). Estimations of percentages of upregulation in the tissues analyzed is shown in FIG. 5. C20orf126 shows significant upregulation (in more than 50% of analyzed pairs) in tissues derived from cancers of the breast, uterus, ovary, colon and rectum.

In order to functionally examine whether C20orf126 could be causally involved in the progression of tumor progression, it was transiently overexpressed or transiently downregulated by RNA interference in HEK-293T cells and subsequently potential resulting influences on tumor cell properties were assayed. For its overexpression a sequence corresponding to the NCBI reference sequence (http://www.ncbi.nlm.nih.gov/RefSeq/) was used. Experiments as previously exemplified for SEQ ID NO:1 in FIGS. 6-8, demonstrate that also overexpression of C20orf126 leads to increased proliferation, its downregulation results in decreased proliferation. C20orf126 also affects invasion potential of tumor cells, as observed in experiments performed according to those exemplified for SEQ ID NO:1 in FIG. 9.

In summary, C20orf126 shows upregulation in metastasizing tumor cells versus non metastasizing tumor cells, and also displays upregulated expression in various tumor tissues versus normal tissue samples. Moreover, C20orf126 is functionally involved in processes involved in tumor progression like increased proliferation and invasion. Therefore, this sequence may particularly be useful for staging of human tumor diseases, as well as for decisions on prognosis and treatment modalities. Furthermore, the C20orf126 gene and its gene products may be used as target structures to develop therapeutic anti-cancer drugs.

SEQ ID NO: 5 (NM_030815)
accgttcttttaactgcgcaggcgcgccggaagcacctagagagcggcgcgtgcgcagcgggagtcgaagcggagatccc
ggggtcgcgcgagagccgcaagcggagttggtgggcgctatgctatcacccgaggcagagcgagtgctgcggtaccttgt
agaagtggaggagctcgccgaggaggtgctggcggacaagcggcagattgtggacctggacactaaaaggaatcagaatc
gagagggcctgagggccctgcagaaggatctcagcctctctgaagatgtgatggtttgcttcgggaacatgtttatcaag
atgcctcaccctgagacaaaggaaatgattgaaaaagatcaagatcatctggataaagaaatagaaaaactgcggaagca
acttaaagtgaaggtcaaccgcctttttgaggcccaaggcaaaccggagctgaagggttttaacttgaaccccctcaacc
aggatgagcttaaagctctcaaggtcatcttgaaaggatgagactcaagaaccaagatgggggaccagcaaccccccagg
gtcatggaggacccaggaccctccaaccttgacacctgtaaggacaggatctgccctgtaaggggccagccgtcaggaat
ctggccatgaaaacctctttgtagtgcttggctactctgtgatggcaggagggaaccttcagcctgtctggctgctggac
ctggacaccagggctcggtggacacaagatctattgacgggccttggtagccaccagtgggtgtgtggggcagtggctgt
gggggtgtaagaatgactgcaacaggcacttcccaacaatggcctgctgttcacatggaccctgagcaaggaaggaggga
gggaggggcagagtggagtgtcattccagcattcctctcagaagggagagaggttttcaggctggtgccatgcgattgga
ataaagcaggaggctcatgggtggttgctgaatgaagaacagaatcttggtgctttgtggctcaccacagccatctgtgg
ggcaggcacacacacctcccgccagctccaattttgcactttttccctgcttgattccaagagtaggtgctgcctagcag
cccttcgtggccactctttactcaggagggccttgcagagtcctgcaccaggcctgggtgagtggatgcgcctcttacca
tatgacacgtgtcaagatgcccttccgccccctctgaaagtggggcccggccagcactgctcgttactgtctgccttcag
tggtctgaggtcccagtatgaactgccgtgaagtcaaaactcttatgtgttcattaagggctcaataaatgttagctgaa
tgaatgaatagcaaaaaaaaaaaa
SEQ ID NO: 14 - PROTEIN (NP_110442, c20orf126)
MLSPEAERVLRYLVEVEELAEEVLADKRQIVDLDTKRNQNREGLRALQKDLSLSEDVMVCFGNMFIKMPHPETKEMIEKDQD
HLDKEIEKLRKQLKVKVNRLFEAQGKPELKGFNLNPLNQDELKALKVILKG

### EXAMPLE 6

SEQ ID NO:6 (H5)

Another rat cDNA clone, originally derived from the above described SSH analysis of the mammary tumor test system was used for a blast analysis against rat EST databases. Similarity was found to the EST BE101513 which the was used to establish the corresponding EST cluster from rat EST databases. Identity within the cluster was over 96%. The consensus sequence of this cluster was used for blast analysis against the human genome browser BLAT (http://genome.ucsc.edu/cgi-bin/hgBlat?command=start). An identity of 90% was found to the human mRNA AK025697 representing the locus FBXO45 which maps on chromosome 3. According to AceView, this gene produces, by alternative splicing, 3 different transcripts altogether encoding 3 different protein isoforms. The corresponding NCBI Reference sequence XM_{—}117294 comprises 4159 nucleotides and encodes a hypothetical protein of 286 amino acids. Comparison to the InterPro Database, a database of protein families, domains and functional sites (http://www.ebi.ac.uk/interpro/index.html), a Cyclin like F box motif is identified in the product of this gene. The F-box domain was first described as a sequence motif found in cyclin-F that interacts with the protein SKP1. This relatively conserved structural motif is present in numerous proteins and serves as a link between a target protein and a ubiquitin-conjugating enzyme. According to InterPro, also the SPIa/RYanodine receptor SPRY motif is found in 2 isoforms from this gene. The SPRY domain is of unknown function.

In FIG. 1, a summary of established data for SEQ ID NO:6 is presented.

This sequence was shown to be differentially expressed in Microarray Analysis comparing samples of metastasizing versus non metastasizing tumor cells as previously exemplified for SEQ ID NO:1 in FIG. 2. Tumor specific expression was further analyzed in hybridization experiments using Cancer Profiling Arrays (CA) from Clontech (http://www.bdbiosciences.com). Estimations of percentages of upregulation in the tissues analyzed is shown in FIG. 5. FBXO45 shows significant upregulation (in more than 50% of analyzed pairs) in tissues derived from cancers of the uterus, ovary, colon and rectum.

In order to functionally examine whether FBXO45 could be causally involved in the progression of tumor progression, it was transiently overexpressed or transiently downregulated by RNA interference in HEK-293T cells and subsequently potential resulting influences on tumor cell properties were assayed. For its overexpression a sequence corresponding to the NCBI reference sequence (http://www.ncbi.nlm.nih.gov/RefSeq/) was used. Experiments as previously exemplified for SEQ ID NO:1 in FIGS. 6-8, demonstrate that also overexpression of FBXO45 leads to increased proliferation, its downregulation results in decreased proliferation. FBXO45 also affects invasion potential of tumor cells, as observed in experiments performed according to those exemplified for SEQ ID NO:1 in FIG. 9.

In summary, FBXO45 shows upregulation in metastasizing tumor cells versus non metastasizing tumor cells, and also displays upregulated expression in various tumor tissues versus normal tissue samples. Moreover, FBXO45 is functionally involved in processes involved in tumor progression like increased proliferation and invasion. Therefore, this sequence may particularly be useful for staging of human tumor diseases, as well as for decisions on prognosis and treatment modalities. Furthermore, the FBXO45 gene and its gene products may be used as target structures to develop therapeutic anti-cancer drugs.

SEQ ID NO: 6 (XM_117294)
gtgcgcccttgcttcgtgccctcaacccgcatggcggagccgctggcgcgccgcggagaggccgggcgagtcgggcggtt
tcggcgcccgcgctgagccgcggaggaggggcggaggacgcccctgcagccggtgcgtctgccctcagtgaggcggggcg
cgcggcggacgcccccgggcaggggcgggagtggtggaggcgccggcggttggcactgacaggggcggtgagcgagccgc
tccggtctccgggcgaggcttggccttccgagcagagacggcgggaagcggcggcggcagcggcggccctagggccggct
ggtgaggcgatggcggcgccggccccgggggctggggcagcctcgggcggcgctggctgtagcggcggcggcgcgggcgc
gggcgcgggctcgggctctggggccgcgggggccgggggccggctgcccagccgggtgctggagttggtgttctcttacc
tggagctgtccgagctgcggagctgcgccctggtgtgcaagcactggtaccgctgcctgcacggcgatgagaacagcgag
gtgtggcggagcctgtgcgcccgcagcctggcagaagaggctctgcgcacggacatcctgtgcaacctgcccagctacaa
ggccaagatacgtgcttttcaacatgccttcagcactaatgactgctccaggaatgtctacattaagaagaatggcttta
ctttacatcgaaaccccattgctcagagcactgatggtgcaaggaccaagattggtttcagtgagggccgccatgcatgg
gaagtgtggtgggagggccctctgggcactgtggcagtgattggaattgccacaaaacgggcccccatgcagtgccaagg
ttatgtggcattgctgggcagtgatgaccagagctggggctggaatctggtggacaataatctactacataatggagaag
tcaatggcagttttccacagtgcaacaacgcaccaaaatatcagataggagaaagaattcgagtcatcttggacatggaa
gataagactttagcttttgaacgtggatatgagttcctgggggttgcttttagaggacttccaaaggtctgcttataccc
agcagtttctgctgtatatggcaacacagaagtgactttggtttaccttggaaaacctttggacggatgacagtggcttt
cttgtgatgacagacagaatggaggagagatctgcttatgggaagtagaaccatgaagtgactgtcacacatgcatgtcc
aagaaacatcctgaaaacacatgaagtcgtaaactggagaagcagctctacagcagagattatcttcgtgtttcctcttt
ctactgggccagaaaaatcctcagggttgcagttggttgagtgggcagttgacatatgcatgttgcacccgatgttgtct
ctaagttagcaatgtgttatttccagctttaaaggtgagattgtagagatgctgtcaaagggataaggaaatagcaagat
ttttaagtagtgtgtttgtgaagactgatcccattttacaactgcctgttctttctccagtccttttttttccagccagc
ttgactattagaaaagtatgaaactggttgggttttatttaatatttttaatatattgagaagcatggtctgcctggact
gcacttctctaaaagtgagatataaaattgtgcagctattttaaaagttgtatataatatgtgtgtaaaaaaaaaaaact
gtaaaaaagaaaggacaaacaggttgttttgttctagttctaatttcttaaaaaccactacatggttacaaaattggaat
aacatttggggacaactgggttaactacaaagaagaggattttaagaggagatgtgttgtattgactcattttgtattat
ttttggcttacagttcccatagctgttagagtctggtttgtttttgtttttactctcaaaatcatagtaaagatctctca
gtctcctggctaaagattgaaggaaggcaaatctatttctaattatacatatatcagtaaggatgatctcaacataatag
taatgtgtatcttttggtatccagttttatttttggccttctaagaaagtgtctcataacacagaacattgccatttgct
cttgtaggcctcaaatatgaaagctattagtcatagagcctaggaaaaaaagaattgattaatggtccttttattttgta
accttataaatgctgtagatattatcaaaaaaattttaatttcatattgtttacatcatgcaactaatctaagcctcaaa
ctcgttattggggctataaagaaaacgtttacttacccagctgaaacaggttaagaatattcttaatctcattatagata
attgcccccatgggacttgaaatacaacaccttgtgctgaaaacttcaggttggcaatatttgaaggtttcgttgtagaa
gagtttaacattaactcctattttgacttacaaatcttgtttctcatcactaaaatgcttttgaattaataatccaaccc
acatgagctgagagtttttcttttgttagaaaagaaacagacatctttctgtatgaaagtataaattgtatggttttaga
tacataagaattgacaaaagcgagcgaaatctttgtacttctgagttcttgctgtatgtatgttttgttttaaatctgat
tagggacacccagcagctggccgggattcttggattgctccttgggagttaagattgtcaatactcctgtgaagcaaggg
atttcagccatagaacaaagatttattgttgccacctgaaaagtttacaagtatttattgtgtatttgatacattgcttg
aaaagatgaaatctgttaaagattcttttcgatgtccaggttaagaagaaacctccttgtattgagtgaaattatatgtt
aaatgtattagagaatgtaggtggtatagaaattgatttttcttggtgtagaacaactcagttcggcaaagtttaaaatt
tgattaaacaagagaagtggttcaggttgaagatggacttgttaggaagtgatcaagtcctttaagtacttgtttctttt
tcaggttgtgatgtggccattccgaattttgttgagagtttggtttataattgtctcttttgtcttgttagtaaacattc
atttgcaacagttttgaaggtgctgagtggaaaaccgaaacacatggttattgcgtattggacctagaatgaaataattg
cctcaatatttaacaacaagccattcttatctcaaagatttaaattcccgaatgtcccattcgcaaatcatatgcaattg
aagtgagcagcatgagcatctgggtcatgagggccttcatttacgtaaatttgtcactaaaacccagtagtagctctaca
aaatcttaaactgctgcagtgctcaaggagatggaatatctttgtcattggtgctgaggagagcatttcggtagaagaca
gttgcgcctgaagattgagtgtaaatcattcaaaccagtggttctcagtgttggctgtatacactttgtagtcactttgg
aatgttggaagacacatcgatgcttgggttccgtatgccaagattctgatgttggtctggaatatgagctggtcataagg
atttttaaaaactttctggtcatttcaatatgctgccaaggttgagaaccactgttgtaaaattcaccttgagttttctc
atctgcaaaatagaaaaaaaaaaatccttgctccctcccttcactacctcacaaggatattgagggtaaaggagaaaata
atgggaaagtgcttgtgccgtggatgaaaagtgctattaaaagtcaaaggagtgttctgtttcaattcatagtatgatca
gggaaagtgtaactgagtatactttgttgacttgggaaacctggagcactttctttggttggttaacgaagcatgcagat
gtggaagcagacgttactattatccctactatggtcttctgtcatactgagacaggctgttttaattacctggttttaca
taggaaagaagaaatattaaggcttaaagtttgtaatgatcaatggctcataattcattaaatcttttcatacaaggaa
SEQ ID NO: 15 - PROTEIN (XP_117294)
MAAPAPGAGAASGGAGCSGGGAGAGAGSGSGAAGAGGRLPSRVLELVFSYLELSELRSCALVCKHWYRCLHGDENSEVWRSL
CARSLAEEALRTDILCNLPSYKAKIRAFQHAFSTNDCSRNVYIKKNGFTLHRNPIAQSTDGARTKIGFSEGRHAWEVWWEGP
LGTVAVIGIATKRAPMQCQGYVALLGSDDQSWGWNLVDNNLLHNGEVNGSFPQCNNAPKYQIGERIRVILDMEDKTLAFERG
YEFLGVAFRGLPKVCLYPAVSAVYGNTEVTLVYLGKPLDG

### EXAMPLE 7

SEQ ID NO:7 (G2)

Another rat cDNA clone, originally derived from the above described SSH analysis of the mammary tumor test system was used for a blast analysis against rat EST databases. Identity of 99% was found to the rat mRNA CO568861. This sequence was used for a blast analysis against human genome databases. An identity of 84% was found to the human mRNA AK025571 representing the locus FLJ21918 which maps on chromosome 16. According to AceView, this gene produces, by alternative splicing, 7 different transcripts altogether encoding 8 different protein isoforms. The corresponding NCBI Reference sequence NM_{—}024939 comprises 4021 nucleotides and encodes a hypothetical protein of 717 amino acids. According to InterPro, the RNA-binding region RNP-1 (RNA recognition motif motif is found in 5 isoforms from this gene. Many eukaryotic proteins that are known or supposed to bind single-stranded RNA contain one or more copies of a putative RNA-binding domain of about 90 amino acids. This is known as the eukaryotic putative RNA-binding region RNP-1 signature or RNA recognition motif (RRM). RRMs are found in a variety of RNA binding proteins, including heterogeneous nuclear ribonucleoproteins (hnRNPs), proteins implicated in regulation of alternative splicing, and protein components of small nuclear ribonucleoproteins (snRNPs). The motif also appears in a few single stranded DNA binding proteins.

In FIG. 1, a summary of established data for SEQ ID NO:7 is presented.

This sequence was shown to be differentially expressed in Microarray Analysis comparing samples of metastasizing versus non metastasizing tumors cells as previously exemplified for SEQ ID NO:1 in FIG. 2. Tumor specific expression was further analyzed in hybridization experiments using Cancer Profiling Arrays (CA) from Clontech (http://www.bdbiosciences.com). Estimations of percentages of upregulation in the tissues analyzed is shown in FIG. 5. FLJ21918 shows significant upregulation (in more than 50% of analyzed pairs) in tissues derived from cancers of the uterus and ovary.

In order to functionally examine whether FLJ21918 could be causally involved in the progression of tumor progression, it was transiently overexpressed or transiently downregulated by RNA interference in HEK-293T cells and subsequently potential resulting influences on tumor cell properties were assayed. For its overexpression a sequence corresponding to the NCBI reference sequence (http://www.ncbi.nlm.nih.gov/RefSeq/) was used. Experiments as previously exemplified for SEQ ID NO:1 in FIGS. 6-8, demonstrate that also overexpression of FLJ21918 leads to increased proliferation, its downregulation results in decreased proliferation. FLJ21918 also affects invasion potential of tumor cells, as observed in experiments performed according to those exemplified for SEQ ID NO:1 in FIG. 9.

In summary, FLJ21918 shows upregulation in metastasizing tumor cells versus non metastasizing tumor cells, and also displays upregulated expression in various tumor tissues versus normal tissue samples. Moreover, FLJ21918 is functionally involved in processes involved in tumor progression like increased proliferation and invasion. Therefore, this sequence may particularly be useful for staging of human tumor diseases, as well as for decisions on prognosis and treatment modalities. Furthermore, the FLJ21918 gene and its gene products may be used as target structures to develop therapeutic anti-cancer drugs.

SEQ ID NO: 7 (NM_024939)
ggtagccgccccgccccgcggggcgccacgggcgggtcttggcagcgcccactgagccagccgggccgcaggtgccgccc
ccgatacacggtgtcccgcccaagctgatccgcgtctgcggtcggtcggtgcgtgcgtgcgcctcgtcggtccgcgtgtc
tggccgagagcccccttcctctgcggccatgactccgccgccgccgccgccccctcccccgggccctgaccccgcggccg
accccgccgcggacccctgcccctggcccggatcactggtcgtcctcttcggggctacggcgggtgcgctgggacgggac
ctgggctcggacgagaccgacttaatcctcctagtttggcaagtggttgagccgcggagccgccaggtggggacgctgca
caaatcgctggttcgtgccgaggcggccgcactgagtacgcagtgccgcgaggcgagcggcctgagcgccgacagcctgg
cgcgggcagagccgctggacaaggtgctgcagcagttctcacagctggtgaacggggatgtggctttgctgggcgggggc
ccctacatgctctgcactgatgggcagcagctattgcgacaggtcctgcaccccgaggcctccaggaagaacctggtgct
ccccgacatgttcttctccttctatgacctccgaagagaattccatatgcagcatccaagcacctgccctgccagggacc
tcactgtggccaccatggcacagggtttaggactggagacagatgccacagaggatgactttggggtctgggaagtcaag
acaatggtagctgttatcctccatctactcaaagagcccagcagtcaattgttttcgaagcccgaggtgataaagcagaa
atacgagacggggccttgcagcaaggctgatgtggtggacagtgagactgtggtacgggctcgtgggttgccgtggcagt
catcagaccaggacgtggctcgcttcttcaaagggctcaacgtggccaggggtggtgtagcactctgcctcaacgcccag
ggccgcagaaatggcgaggccctcatccgctttgtggacagcgagcagcgggacctagcgctgcagagacacaagcacca
catgggcgtccgctatattgaggtgtataaagcgacaggggaggagtttgtaaagattgcagggggcacatcactagagg
tggctcgtttcttgtcacgggaagaccaagtgatcctgcggctgcggggactgcccttctcggctgggccaacggacgtg
cttggcttcctggggccagagtgcccagtgactgggggtaccgaggggctgctctttgtgcgccatcctgatggccggcc
gactggtgatgccttcgccctctttgcttgtgaggagctggcacaggctgcactgcgcaggcacaagggcatgctgggta
agcgatacattgaactcttccggagcactgcagccgaagtgcagcaggtcttgaaccgctatgcatccggcccactcctt
cctacactgactgccccactgctgcccatccccttcccactggcacctgggactgggagggactgtgtacgcctccgagg
cctgccctacacggccaccattgaagacatcctgagctttctgggggaggcagcagctgacattcggccccacggtgtac
acatggtgctcaaccagcagggccggccatcgggcgatgccttcattcagatgacatcagcagagcgagccctagctgct
gctcagcgttgccataagaaggtgatgaaggagcgctacgtggaggtggtcccctgttccacagaggagatgagccgagt
gctgatggggggcaccttgggccgcagtggcatgtcccctccaccctgcaagctgccctgcctctcaccacctacctaca
ccaccttccaagccaccccaacgctcattcccacggagacggcagctctatacccctcttcagcactgctcccagctgcc
agggtgcctgctgcccccacccctgttgcctactatccagggccagccactcaactctacctgaactacacagcctacta
cccaagccccccagtctcccccaccactgtgggctacctcactacacccactgctgccctggcctctgctcccacctcag
tgttgtcccagtcaggagccttggtccgcatgcagggtgtcccatacacggctggtatgaaggatctgctcagcgtcttc
caggcctaccagctacccgctgatgactacaccagtctgatgcctgttggtgacccacctcgcactgtgttacaagcccc
caaggaatgggtgtgtttgtaggagagaaagccaggaggtaagagccagctgatatcctcggcgaacatgtctctcctga
gtccagaagaccagcaccctcaacctggtagcttctttctggcttgtcaaagctctcagaaggtacctagaggagcccaa
gccccagctccatcctccacttattctgcctgtttcccccaaagacaatggctggaccctgcatgcagggctgggggtgg
aatggggctaaccagctcctgatggcctgagccaggcatcttgactggcacctggagagcccttaagtctgtcctggctg
tggcccatgccgacagatatcgtggggctgacaggtccacggcaggcttgctttcttttataaaatggaagctctggtac
cttcaatgtatgactcctgggagaatcaagggtccatctgagcctctgagtaaagatcccaatgttctacctctccctgt
ccctcttgtaggggatagggaggcagagagagccagcccctaccctcagagtatctggacctcagagaccatgttgtgcc
aggggtggtcccacctaaagatgctagcccctctccaggtgggcataaggagtaacagatggcaaaaccacaaactattt
tgatggactgtgctgcagtatcaccagaagacattagggggcagtaggcccccacacaaaaccttcaggcttgaatttta
aaggggaggactttctgccaacttttcttgtatgccttgggaaagccagttgccctgaacccagcagacaccatggaatg
tcctttgcacgcattaaatggtacagaactgaagcctcggaagcaatttggaactcgatcttctcttccttaaatgaaaa
gttattgaccaaatggactttttaaaagacacaggacccttaactttgccccaaagtgaggggctccacaccaaccccag
gcggaggaacactcagacagattaaggatactgttgacctgtcactgtttattatttcagcactaaaactgaggagcctc
aactgctggctcttcttccctttgtatttgtgtaaggagcactgcactcccataaaaggttttaaaatacaaaatgtaca
agaacacacaattccaagtgctgtaaacataactgagaaccagttcctttactaaacatccattttataaaacacaaggt
ttcaatttgagcccatctgagccttaaagatccattctgaataccaaaaacagggcttcacagccaggcccagaagaggt
ctggtgataatggctggccctgggtggggatagtttacacccgggcagcagcaccacacatgaacccaaagacatgttct
ttttaaagctgttttcagccatgtttctctgtgcatctccagtaagcagaaggctacccattccattcctcaacccaaga
gctagcacagttagagtaggagggggtgcgtactagcacgtgcccagttgctcagtgctgctagtagaaattgatttgca
tagtccaatggatgtgtgctttaacaccactatgttgcacaaaaatttaagtctttatctacaaagccaaaaaatattga
ctcttaacaccaaagcttttacaaagctgatataaaactgcttacatagtatacaaagctctattttaaaatttaatgtt
tattttaaataggaaagcatt
SEQ ID NO: 16 - PROTEIN (NP_079215)
MTPPPPPPPPPGPDPAADPAADPCPWPGSLVVLFGATAGALGRDLGSDETDLILLVWQVVEPRSRQVGTLHKSLVRAEAAAL
STQCREASGLSADSLARAEPLDKVLQQFSQLVNGDVALLGGGPYMLCTDGQQLLRQVLHPEASRKNLVLPDMFFSFYDLRRE
FHMQHPSTCPARDLTVATMAQGLGLETDATEDDFGVWEVKTMVAVILHLLKEPSSQLFSKPEVIKQKYETGPCSKADVVDSE
TVVRARGLPWQSSDQDVARFFKGLNVARGGVALCLNAQGRRNGEALIRFVDSEQRDLALQRHKHHMGVRYIEVYKATGEEFV
KIAGGTSLEVARFLSREDQVILRLRGLPFSAGPTDVLGFLGPECPVTGGTEGLLFVRHPDGRPTGDAFALFACEELAQAALR
RHKGMLGKRYIELFRSTAAEVQQVLNRYASGPLLPTLTAPLLPIPFPLAPGTGRDCVRLRGLPYTATIEDILSFLGEAAADI
RPHGVHMVLNQQGRPSGDAFIQMTSAERALAAAQRCHKKVMKERYVEVVPCSTEEMSRVLMGGTLGRSGMSPPPCKLPCLSP
PTYTTFQATPTLIPTETAALYPSSALLPAARVPAAPTPVAYYPGPATQLYLNYTAYYPSPPVSPTTVGYLTTPTAALASAPT
SVLSQSGALVRMQGVPYTAGMKDLLSVFQAYQLPADDYTSLMPVGDPPRTVLQAPKEWVCL

### EXAMPLE 8

SEQ ID NO:8 (L1)

Another rat cDNA clone, originally derived from the above described SSH analysis of the mammary tumor test system was used for a blast analysis against rat EST databases. 100% identity was found to the rat EST AW919679. This EST was used for a blast analysis against mouse genome databases. Identity of 90% was found to the mouse mRNA AK088107. The protein encoded by this RNA shows 90% identity on the amino acid level to the human hypothetical protein NP_{—}620129 encoded by the locus C19orf22, alias MGC16353. The corresponding NCBI Reference sequence NM_{—}138774 comprises 1810 nucleotides and encodes a hypothetical protein of 166 amino acids. According to AceView, it produces, by alternative splicing, 8 different transcripts altogether encoding 7 different protein isoforms. PSORT II analysis, trained on yeast data, predicts that the subcellular location of this partial protein is expected to be in the nucleus (56%). The following domain was found: PKAKGRK. Pfam analysis shows that this protein does not belong to any recognized protein family.

In FIG. 1, a summary of established data for SEQ ID NO:8 is presented.

This sequence, C19orf22, was shown to be differentially expressed in Microarray Analysis comparing its expression in metastasizing versus non metastasizing tumor cells as previously exemplified for SEQ ID NO:1 in FIG. 2. Tumor specific expression was further analyzed in hybridization experiments using Cancer Profiling Arrays (CA) from Clontech (http://www.bdbiosciences.com). Estimated percentages of upregulation in the tissues analyzed is shown in FIG. 5. C19orf22 shows significant upregulation (in more than 50% of analyzed pairs) in tissues derived from cancer of the uterus, ovary, colon, rectum and lung.

In order to functionally examine whether C19orf22 could be causally involved in the progression of tumor progression, it was transiently overexpressed or transiently downregulated by RNA interference in HEK-293T cells and subsequently potential resulting influences on tumor cell properties were assayed. For its overexpression a sequence corresponding to the NCBI reference sequence (http://www.ncbi.nlm.nih.gov/RefSeq/) was used. Experiments as previously exemplified for SEQ ID NO:1 in FIGS. 6-8, demonstrate that also overexpression of C19orf22 leads to increased proliferation, its downregulation results in decreased proliferation. C19orf22 also affects invasion potential of tumor cells, as observed in experiments performed according to those exemplified for SEQ ID NO:1 in FIG. 9.

In summary, C19orf22 shows upregulation in metastasizing tumor cells versus non metastasizing tumor cells, and also displays upregulated expression in various tumor tissues versus normal tissue samples. Moreover, C19orf22 is functionally involved in processes involved in tumor progression like increased proliferation and invasion. Therefore, this sequence may particularly be useful for staging of human tumor diseases, as well as for decisions on prognosis and treatment modalities. Furthermore, the C19orf22 gene and its gene products may be used as target structures to develop therapeutic anti-cancer drugs.

SEQ ID NO: 8 (NM_138774)
gaaggccctgccgggcggcggcggcggcgacagcgtgcgagccatggtcgcgctggagaaccccgagtgcggcccggagg
cggcggagggcaccccgggcgggcggcggctgctgccccttcccagctgcctgcctgccctagccagctcccaggtgaag
agactctcggcttccaggcggaaacagcacttcatcaaccaggcagtgcggaactcagacctcgtgcccaaggccaaggg
gcggaagagcctccagcgcctggagaacacccagtacctcctgaccctgctggagacagacgggggcctgcctggcctgg
aggatggggacttggcaccccctgcatcaccaggcatctttgccgaggcctgcaacaacgccacctatgtggaggtctgg
aacgatttcatgaaccgctccggggaggagcaggagcgggttcttcgctacctggaggatgagggcaggagcaaggcgcg
gaggaggggccctggccgtggggaggaccggaggagagaggaccccgcctatacaccccgcgagtgcttccagcgcatca
gccggcgtctgcgagccgtcctcaagcgcagccgcatccccatggaaacgctggagacctgggaggagcggctgcttcgg
ttcttctccgtgtccccccaggccgtgtacacagcaatgctagacaacagcttcgagaggcttctgctgcacgctgtctg
ccagtacatggacctcatctcggccagtgctgacctggaggggaagcggcagatgaaggtcagtaatcggcacctggatt
tcctgccgccggggctgctcctgtccgcctacctggagcagcacagctgatggcggccccgcggagaccccgctgccacc
tcgcccagccatcaagccctccgataccttcggctaaaatatctttcatatttttagaatttgtcctcggaaaccttttt
cgcttggggtggtctctctcactctgccccctcctcacgcagctcttggcagtcaacagacgctggcggctggggctgcc
catgccatcccagctccaagcttcccactccgggacttgtgtttgggtggggagacctgacctgggcatgttcctgtttc
ttcatcgttgagcttttctggcccggtctgaagctcaagtgaggagggggaggctgggtttttatcacttttaatgaatt
tggtgtgatttgttgtagatttttaaatttcccttttggagagaaaaaccaaaaaaactcgccccactggtaaaacatgg
gtcttggtcccagcccctgctcagcccctcccagtttttagcttgaatgagggtggggtctctgggaccctgcccctcat
gccagaagcatcttgtgttgtatatgtgtgcgcgcgtgtgccctgagacccaggacagaagccacggtcctaagagccgg
ttttatcctcgtcattctgcgtgtcctcccccacgccacctgtgtcggggctcagggtctcctgctttatatgagccccc
ttcctttcctcccctcctttatgctgggggtccaggacttccagccagaagcctctgcccttgcactaccttgtctgtca
ccccatcccgtgtcccctcgtcccccagcctgactcctgcctgatagctcctgtgtccccatgctggtcctcctggccca
ggctgcaggagccaggctggggggcctccgcacccccttgctgcgtgtgggtaattgtgttttgggggaaagtggggaat
ttaataaatttctggtgctctggcaaaaaaaaaaaaaaaaaaaaaaaaaa.
SEQ ID NO: 17 - PROTEIN (NP_620129)
MVANCGAAGTGGRRSCAASSVKRSASRRKHNAVRNSDVKAKGRKSRNTYTTDGGGDGDAASGAACNNATYVVWNDMNRSGRV
RYDGRSKARRRGGRGDRRRDAYTRCRSRRRAVKRSRMTTWRRSVSAVYTAMDNSRHAVCYMDSASADGKRMKVSNRHDGSAY
HS

### EXAMPLE 9

SEQ ID NO:9 (G4)

Another rat cDNA clone, originally derived from the above described SSH analysis of the mammary tumor test system was used for a blast analysis against rat EST databases. 84% identity was found to the rat RNA BC030338 representing the locus LOC292139. The protein encoded by this locus shows 77% identity to the hypothetical human protein NP_{—}060800 representing the locus KIAA1598. The corresponding NCBI reference mRNA for this locus NM_{—}018330 comprises 3417 nucleotides and encodes a hypothetical protein of 456 amino acids which maps on chromosome 10. According to AceView, this gene produces, by alternative splicing, 11 different transcripts altogether encoding 11 different protein isoforms. PSORT II analysis predicts that the subcellular location of this protein is expected to be in the nucleus (60%). Pfam Search shows that the amino-terminus of the protein shares homology with the SMC domain of Chromosome segregation ATPases.

In FIG. 1, a summary of established data for SEQ ID NO:9 is presented.

This sequence was shown to be differentially expressed in Microarray Analysis comparing samples of metastasizing versus non metastasizing tumor cells as previously exemplified for SEQ ID NO:1 in FIG. 2. Tumor specific expression was further analyzed in hybridization experiments using Cancer Profiling Arrays (CA) from Clontech (http://www.bdbiosciences.com). Estimated percentages of upregulation in the tissues analyzed is shown in FIG. 5. KIAA1598 shows significant upregulated expression (in more than 50% of analyzed pairs) in tissues derived from cancers of the uterus, ovary, colon and rectum.

In order to functionally examine whether KIAA1598 could be causally involved in the progression of tumor progression, it was transiently overexpressed or transiently downregulated by RNA interference in HEK-293T cells and subsequently potential resulting influences on tumor cell properties were assayed. For its overexpression a sequence corresponding to the NCBI reference sequence (http://www.ncbi.nlm.nih.gov/RefSeq/) was used. Experiments as previously exemplified for SEQ ID NO:1 in FIGS. 6-8, demonstrate that also overexpression of KIAA1598 leads to increased proliferation, its downregulation results in decreased proliferation. KIAA1598 also affects invasion potential of tumor cells, as observed in experiments performed according to those exemplified for SEQ ID NO:1 in FIG. 9.

In summary, KIAA1598 shows upregulation in metastasizing tumor cells versus non metastasizing tumor cells, and also displays upregulated expression in various tumor tissues versus normal tissue samples. Moreover, KIAA1598 is functionally involved in processes involved in tumor progression like increased proliferation and invasion. Therefore, this sequence may particularly be useful for staging of human tumor diseases, as well as for decisions on prognosis and treatment modalities. Furthermore, the KIAA1598 gene and its gene products may be used as target structures to develop therapeutic anti-cancer drugs.

SEQ ID NO: 9 (NM_018330)
cgaggctggcatagcggctgccgacccgccttcgttcctccaccccctgcacgggactgctgggcccgccccgccccgcc
tgcaggtgaagcggccgcagccgccgagtaggtgcgtggggatgatctcactcgcgcgctccgcgccaggaggaggagga
gcgggagcggatccaacttccgggtagtggagccgcaagccaccggcatcttgctttttcttccccctcctcctgtgtgc
cccgcgccgctccctctttcccttttattcccggccccacccgccaaaatgaacagctcggacgaagagaagcagctgca
gctcattaccagtctgaaggagcaagcaataggcgaatatgaagaccttagagcagagaaccagaaaacaaaggagaagt
gtgacaaaattaggcaagaacgagatgaagccgttaaaaaactggaagaatttcagaaaatttctcacatggtcatagag
gaagttaatttcatgcagaaccatcttgaaatagagaagacttgtcgagaaagtgctgaagctttggcaacaaagctaaa
taaagaaaataaaacgttgaaaagaatcagcatgttgtacatggccaagctgggaccagatgtaataactgaagagataa
acattgatgatgaagattcgactacagacacagacggtgccgccgagacttgtgtctcagtacagtgtcagaagcaaatt
aaagaacttcgagatcaaattgtatctgttcaggaggaaaagaagattttagccattgagctggaaaatctcaagagcaa
actcgtagaagtaattgaagaagtaaataaagttaaacaagaaaagactgttttaaattcagaagttcttgaacagagaa
aagtcttagaaaaatgcaatagagtgtccatgttagctgtagaagagtatgaggagatgcaagtaaacctggagctggag
aaggaccttcgaaagaaagcagagtcatttgcacaagagatgttcattgagcaaaacaagctaaagagacaaagccacct
tctgctgcagagctccatccctgatcagcagcttttgaaagctttagacgaaaatgcaaaactcacccagcaacttgaag
aagagagaattcagcatcaacaaaaggtcaaagaattagaagagcaactagaaaatgaaacactccacaaagaaatacac
aacctcaaacagcaactggagcttctagaggaagataaaaaggaattggaattgaaatatcagaattctgaagagaaagc
cagaaatttaaagcactctgttgatgaactccagaaacgagtgaaccagtctgagaattcagtacctccaccacctcctc
ctccaccaccacttccccctccacctcccaatcctatccgatccctcatgtccatgatccggaaacgatcccaccccagt
ggcagtggtgctaagaaagaaaaggcaactcaaccagaaacaactgaagaagtcacagatctaaagaggcaagcagttga
agagatgatggatagaattaaaaagggagttcatcttagacccgttaatcagacagccagaccgaagacaaagccagaat
cttcgaaaggctgcgaaagtgcagtggatgaactaaaaggaatactggcctcccagtagcattggatgcaggaaaaaata
cattgacggtgaaaaacaagccgaaccagttgtagttttagatcctgtttctacacatgaaccccaaaccaaagaccagg
ttgctgaaaaagatccaactcaacacaaggaggatgaaggcgaaattcaaccagaaaacaaagaagacagcattgaaaac
gtgagagagacagacagctccaactgctgatccataaaccagaagcctgatacgtttggaagtccttttcaataagcaca
tgattagtgttgttatattggcaagggctgtagacattctgctctggtcactgtattcagaatacaggttcttttctggt
gtcacttttgtaagtagcaactataaacataagtaagctgtttagcaaaacacacattcctagtaggttttggttttttg
atctttataaagatgaggtttttttcctagttactgtattaagtatgacttcttttagaaggttacaaaaaaattcagat
gttgatacctttttaggaaatgtgcataccactcatcaaatggaatgctgaaagtttgaggtgcttgtatataatcggat
aaacaaaactgatcaacccaatgtgattttaaaagcccccaaagaagcttctgttttgggtctgatcctcttgatggaga
aactgcagcagcatggaaattgttgggtactgtggcatacaagttattttctacagtagactgagataaactgaaaactc
aggagctggcatcaaactcgtagtcccatagtcagtgttaattacacacattgttaactattggatgaaaaatacatgct
attgattgtgtccaaagcctcccgaggacctccgtggggatgctctggtagcctgaatacagaactgaggtgaaagtcca
aaccttgaattttacagtagtaagttggtaaaccatgtgctctgtgctatgagttaattatgttttcccaaatactaatg
tggcacaagtaccatattttatcagagttcttatgtacagtatggtgaagataagtgacaagcacacatttttcttgctt
cactgctgttctatattacacaggtttgttgttgttttttttaaaaaagaaattaagcagtagttagtctctaaaaatac
aatgtttcaggctaccacagtgaataaatagaaatgtaatcagggattaaaaaaaaaacttatgcagcttttcaaagttg
attgtttcaaaattggtgtttatttaaaataagtggtaatgtacttgaatgcactttttatgacaatgattcagtaatgg
taattttactattaaagaaagtgaaaggtttagttttgttagcatggctcagcatgtagctgtcaggtgtttttcaccta
agggcaaaagaaaatgatagtaataattgcagtagttgtattgtattgtatttttgcacgtgtggtaagcataggcttga
agaggtgggtaggcaggtacatgtacttcctaaattttgagataattatctttctgtaagttcgttatgcttgactgttt
ccatgttctcccaataatgattttatagttacttatcactttactcatggagaattaaaacgtaatgtttttcaactgta
tctttctttaactggataatactgctatatgatatgcttactacagactgcattaattcacgaaacgaattctgttatgc
tgtaatttgaactctcctcaccacaacttattaaaaaggcaccaatagtttcccatt
SEQ ID NO: 18 - PROTEIN (NP_060800)
MNSSDEEKQLQLITSLKEQAIGEYEDLRAENQKTKEKCDKIRQERDEAVKKLEEFQKISHMVIEEVNFMQNHLEIEKTCRES
AEALATKLNKENKTLKRISMLYMAKLGPDVITEEINIDDEDSTTDTDGAAETCVSVQCQKQIKELRDQIVSVQEEKKILAIE
LENLKSKLVEVIEEVNKVKQEKTVLNSEVLEQRKVLEKCNRVSMLAVEEYEEMQVNLELEKDLRKKAESFAQEMFIEQNKLK
RQSHLLLQSSIPDQQLLKALDENAKLTQQLEEERIQHQQKVKELEEQLENETLHKEIHNLKQQLELLEEDKKELELKYQNSE
EKARNLKHSVDELQKRVNQSENSVPPPPPPPPPLPPPPPNPIRSLMSMIRKRSHPSGSGAKKEKATQPETTEEVTDLKRQAV
EEMMDRIKKGVHLRPVNQTARPKTKPESSKGCESAVDELKGILASQ

### EXAMPLE 10

All clones were used to perform blast analyses using gene sequence databases. Out of these investigations, in summary, 89 of 235 deduced human sequences were chosen and corresponding cDNAs were spotted with 4 ng per spot onto glass slides (Cornings CMT ULTRAGaps slides), to create a diagnostic, a so called cDNA chip. Subsequent hybridization experiments showed that all of these 89 sequences are differentially expressed in at least one of several pairs of metastasizing and non metastasizing cells, such as, e.g., in five pairs of primary tumor and metastasis samples from colon cancer patients.

In addition, the expression patterns of these 89 sequences in established cell lines displaying different metastasizing potentials were analysed. The following cell lines were utilized for this purpose:
The non metastasizing colon cancer cell line SW480 and the metastasizing colon cancer cell line SW620 (ATCC CCL-227 and -228).
The non metastasizing colon cancer cell line HT29mtx and the metastasizing colon cancer cell line HT29 (Lesuffleur, 1990, Cancer Res. 50, 6334-6343).
The non metastasizing mammary cell line T47D and the metastasizing mammary cancer cell line MDA-MB-231 (ATCC HTB-133 and -26).
The non metastasizing endometrial cancer cell line HEC-1A and the metastasizing endometrial cancer cell line AN3-CA (ATCC HTB-112 and -111).
The non metastasizing prostate cancer cell line LNCap and metastasizing prostate cancer cell line DU145 (ATCC HTB-81 and CRL-1740).
The non metastasizing pharynx carcinoma line FaDu and the Detroit-562 line established from a metastatic site of a pharynx carcinoma (ATCC HTB-43 and CCL-138).

Accession numbers of all sequences that showed differential expression at least in one of these systems in microarray analysis are listed in FIG. 1 which also contains information on differential expression of the single sequences established by “In situ hybridisation” (ISH) technology of matched human tumors (BioCat BA3, http://www.biocat.de). Three sequences were tested for their expression patterns on these slides and showed tumor specific expression in at least two tissue types. Tumor specific expression patterns were further analyzed in hybridization experiments using Cancer Profiling Arrays (CA) from Clontech (http://www.bdbiosciences.com). These Cancer Profiling Arrays include normalized amplified cDNAs from 241 tumor tissues and corresponding normal tissues from individual patients, along with negative and positive controls, and also cDNAs from nine cancer cell lines. In these experiments, overexpression of a given gene in these Cancer profiling Assays was defined as upregulation of expression in the tumor probe versus the normal probe in at least 50% of analyzed pairs which were analysed in at least 3 of 8 different tissues analysed. 25 of the 89 sequences listed in FIG. 1 were tested in the Cancer profiling Arrays; 9 of those showed tumor specific expression patterns according to the above mentioned criteria. Furthermore, FIG. 1 contains information on indications for functional involvement of the sequences listed in metastatic processes. A positive mark in this context was defined as displaying an at least 20% modification of activity over control values in at least one functional assay. For further detailed information on functional assays performed see FIGS. 6-9.

An example of a gene-chip hybridization experiment utilizing cDNAs from the endometrial cancer cell line HEC-1A and the metastasizing endometrial cancer cell line AN3-CA (ATCC HTB-112 and -111) is shown in FIG. 2.

In summary, all sequences listed in FIG. 1 display metastasis specific expression patterns in hybridisation experiments. 9 of these sequences (designated SEQ ID NO:1-9) were tested positive for 2 further criteria of causal relevance and their involvement in the process of tumor progression.

These findings show, that this cDNA chip comprising the listed sequences of FIG. 1 can be used as a diagnostic and prognostic tool. It will enable the investigator to conclude about the presence of metastatic tumor cells in the body of a patient, and furthermore, might predict in future the therapeutic outcome of a given therapy, given that the therapy interferes with the presence or absence of one or several of the molecular cancer antigens presented in this invention and represented as cDNA on the corresponding diagnostic cDNA chip described above. In case a cancer antigen directly represents an anti-cancer target structure, than the therapeutic outcome might directly be measurable based on the activity or expression of this cancer antigen, e.g., if this cancer antigen is attacked therapeutically directly or indirectly by the therapeutic agent.

A therapeutic modulation of a cancer antigens function could be established by interfering with the expression of such a cancer antigen by e.g., including but not limited to, utilizing means of anti-sense RNA, RNAi or catalytic RNA technologies, or by various DNA or modified DNA oligonucleotide approaches.

Alternatively, antibodies directed against these cancer antigens could be suitable anti-cancer drugs, or drugs that interfere with activities, such as, but not limited to, enzymatic or structural activities, of these cancer antigens, or their existing localization specifications. Also, drugs which act on signaling pathways which are influenced by these cancer antigens could give rise to potent anti-cancer drugs.

In a particular embodiment of this invention, such therapeutic approaches could be suitable for the treatment of metastatic cancer disease, or for the prevention or suppression of metastatic tumor progression, and for the treatment, prevention and suppression of minimal residual tumor disease.

### FIGURES

FIG. 1: List of cancer antigens identified, characterized and presented in this invention.

FIG. 2: Raw Microarray analysis data from hybridization tests.

FIG. 3: Data of ISH (In Situ Hybridization) experiments with Digoxygenin labelled RNA probes from the MEP 50 locus (SEQ ID NO:1) are presented.

FIG. 4: The cancer profiling expression analysis (CA) for SEQ ID NO:1 (MEP50) is presented.

FIG. 5: Summary data for the cancer profiling expression analysis (CA) for SEQ ID NO:1-9.

FIG. 6: Data from proliferation assays (A) with transiently transfected HEK-293T cells.

FIG. 7: Proliferation assay using siRNA treated HEK-293T cells.

FIG. 8: Proliferation assays using overexpression studies.

FIG. 9: Invasion assay with stably transfected HT29 colon cancer cells.

1. A method for diagnosing a disease or condition, or a susceptibility to a disease or condition, comprising the step of determining the expression, activity or mutations of at least one polynucleotide or expression product thereof in a first biological sample from a first subject, wherein said at least one polynucleotide is selected from the group consisting of:
(i) a polynucleotide having a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9 and the corresponding RNA sequences,
(ii) a polynucleotide having a sequence complementary to any one of the sequences under (i), or
(iii) a polynucleotide variant of any one of the polynucleotides under (i) or (ii), and
(iv) combinations thereof.

2. A method according to claim 1, wherein said at least one polynucleotide comprises a sequence encoding a polypeptide having a sequence selected from the group consisting of SEQ ID NO:10, SEQ ID NO:11, SEQ ID NO:12, SEQ ID NO:13, SEQ ID NO:14, SEQ ID NO:15, SEQ ID NO:16, SEQ ID NO:17 and SEQ ID NO:18.

3. A method according to claim 1, wherein said expression product comprises a polypeptide comprising a sequence selected from the group consisting of SEQ ID NO:10, SEQ ID NO:11, SEQ ID NO:12, SEQ ID NO:13, SEQ ID NO:14, SEQ ID NO:15, SEQ ID NO:16, SEQ ID NO:17 and SEQ ID NO:18.

4. A method according to claim 1, comprising:
determining the expression or activity of said at least one polynucleotide or expression product thereof in said biological sample.

5. A method according to claim 4, wherein said determining the expression of said at least one polynucleotide comprises determining the presence and/or amount of said at least one polynucleotide or expression product thereof in said biological sample.

6. A method according to claim 1, wherein said determining mutations consists of determining the presence or absence of one or more mutations in the nucleotide sequence of said at least one polynucleotide in said biological sample.

7. A method according to claim 1, comprising the use of hybridization technology.

8. A method according to claim 1, wherein said determining expression of at least one polynucleotide in said sample comprises utilizing at least one recombinant polynucleotide.

9. A method according to claim 7, further comprising:
contacting a solid support on which at least one isolated polynucleotide is immobilized with said sample, and the isolated polynucleotide is selected from the group consisting of:
(i) a polynucleotide having a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and the corresponding RNA sequences,
(ii) a polynucleotide having a sequence complementary to any one of the sequences under (i),
(iii) a polynucleotide variant of any one of the polynucleotide sequences under (i) or (ii), and
(iv) combinations thereof.

10. A method according to claim 9, wherein at least 9 different isolated polynucleotides are immobilized on said solid support, and said 9 different isolated polynucleotides have the nucleotide sequences as shown in SEQ ID NO:1, SEQ ID NO:2, SEQ ID NO:3, SEQ ID NO:4, SEQ ID NO:5, SEQ ID NO:6, SEQ ID NO:7, SEQ ID NO:8 and SEQ ID NO:9, respectively.

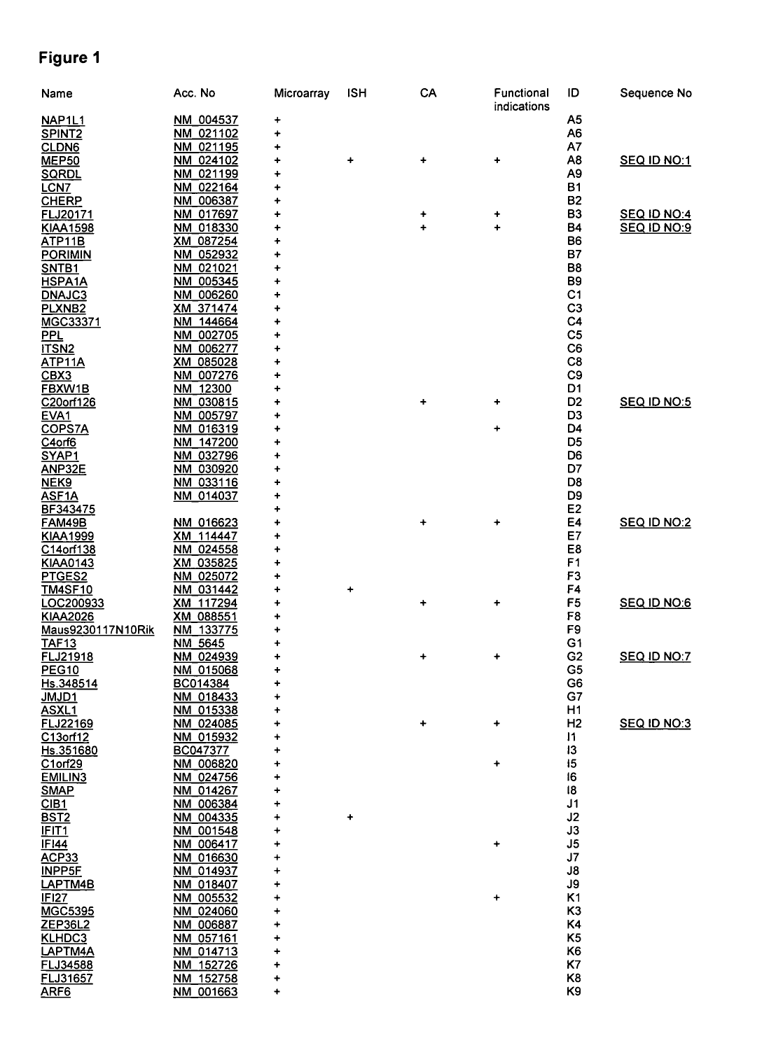

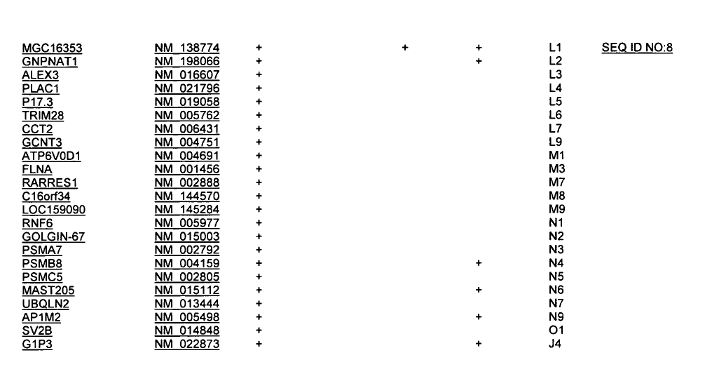

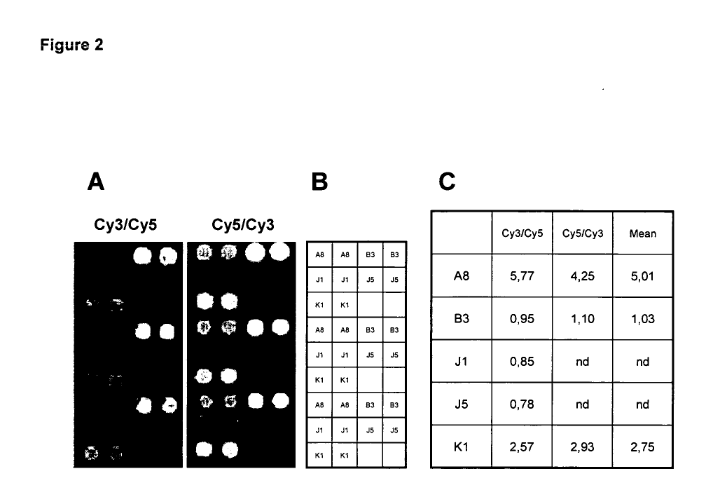

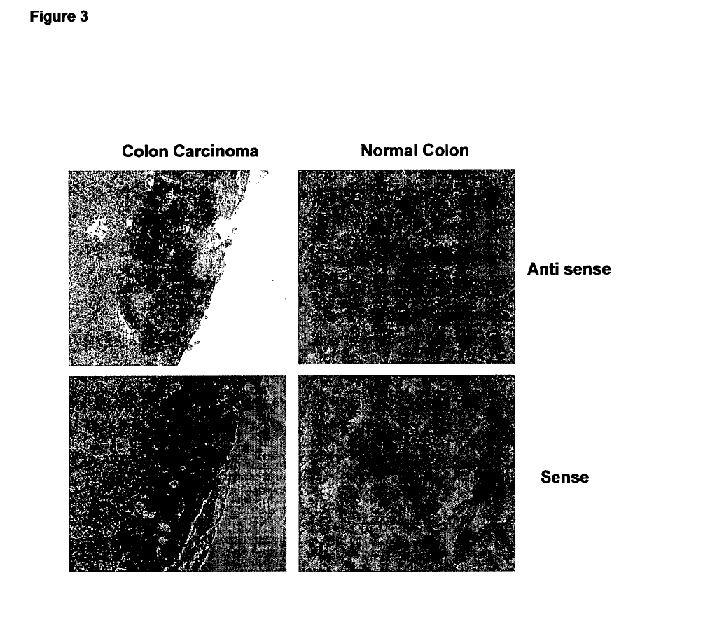

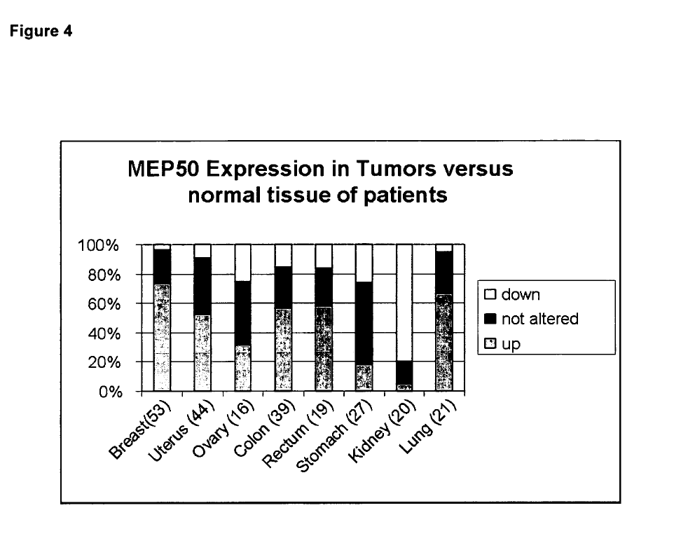

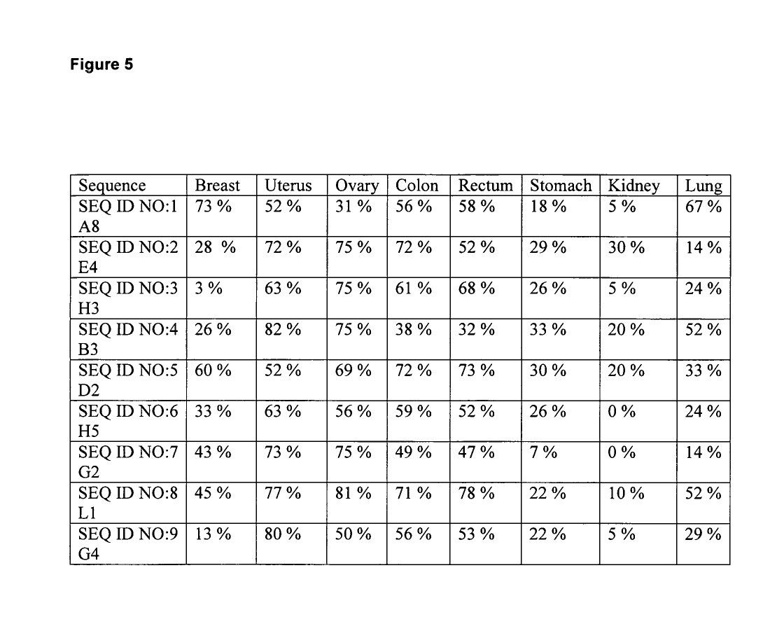

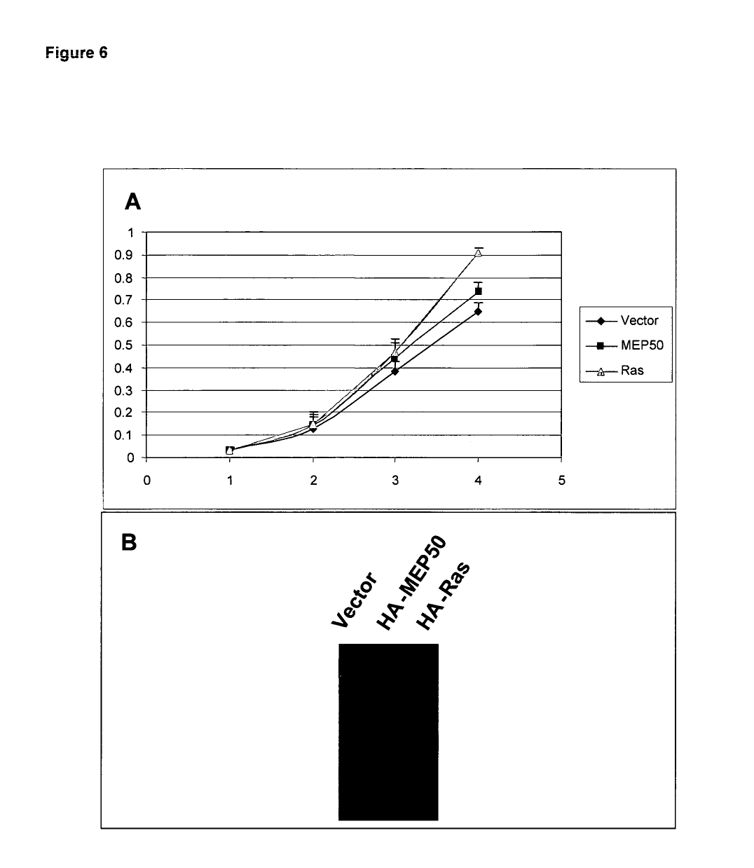

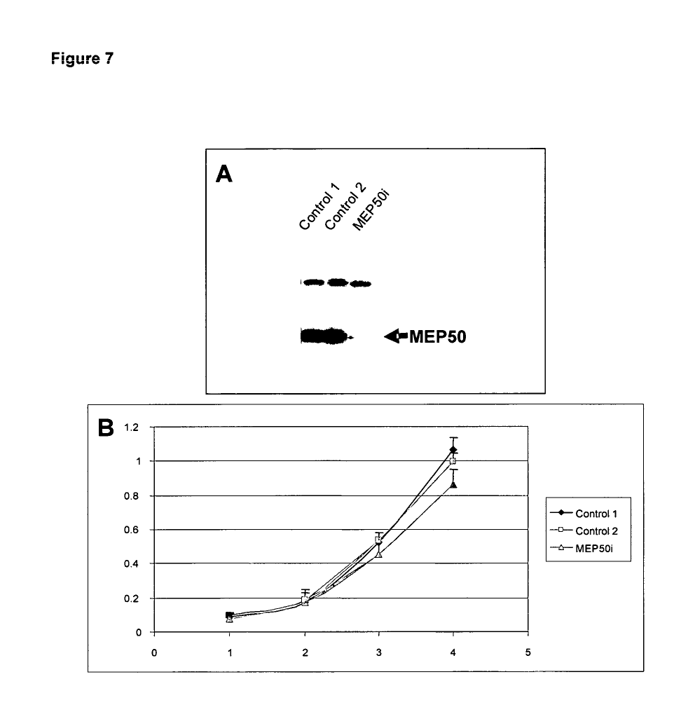

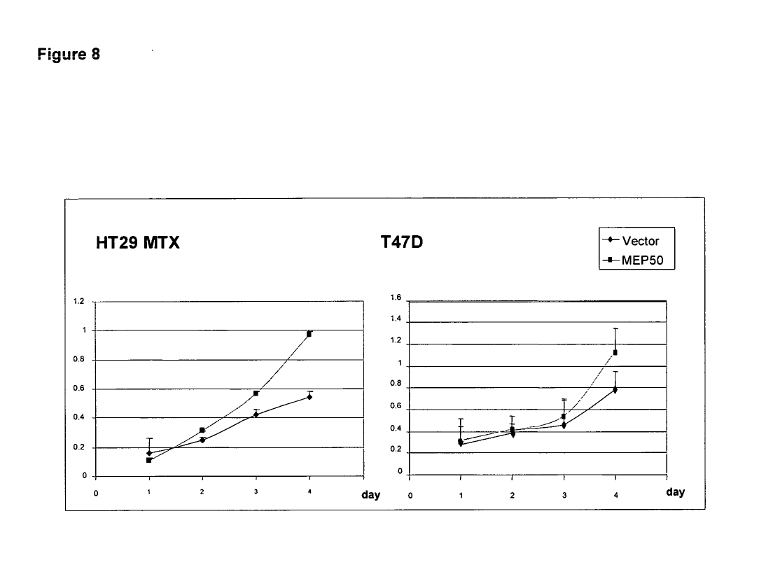

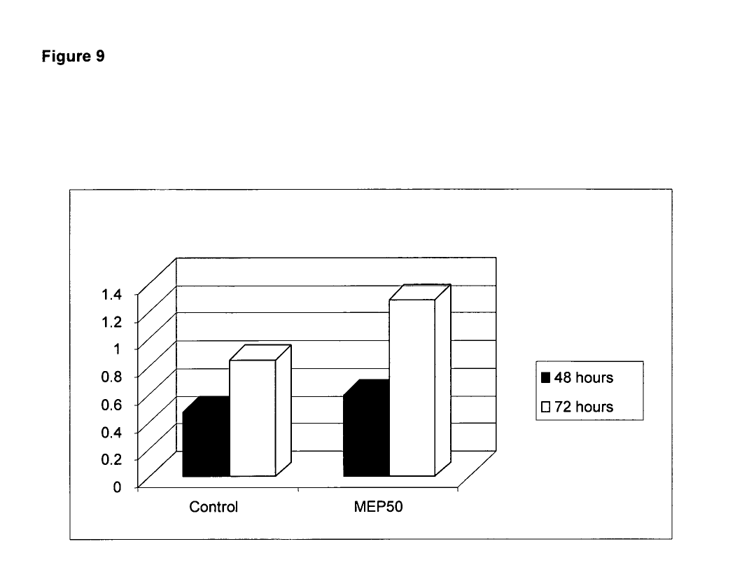

11. A method according to claim 10, wherein at least 89 different isolated polynucleotides are immobilized on said solid support, and said 89 isolated polynucleotides have the nucleotide sequences in FIG. 1.

12. A method according to claim 1, comprising:
utilizing an antibody directed against a polypeptide selected from the group consisting of SEQ ID NO:10, SEQ ID NO:11, SEQ ID NO:12, SEQ ID NO:13, SEQ ID NO:14, SEQ ID NO:15, SEQ ID NO:16, SEQ ID NO:17, and SEQ ID NO:18.

13. A method according to claim 1, further comprising:
comparing said expression or activity in said first sample with the expression or activity of said at least one polynucleotide or expression product thereof in a second sample which was obtained from tissue which is not affected by said disease.

14. A method according to claim 13, further comprising:
determining if expression or activity in said first sample is higher than the expression or activity in said second sample.

15. A method according to claim 1, wherein the disease is a tumor disease.

16. A method according to claim 15, which is a method for testing the presence of tumor cells in the subject's body.

17. A method according to claim 16, which is a method for testing whether the subject's body contains tumor cells with an increased metastatic potential.

18. A method according to claim 1, wherein the disease is selected from the group consisting of estrogen receptor-dependent breast cancer, estrogen receptor-independent breast cancer, hormone receptor-dependent prostate cancer, hormone receptor-independent prostate cancer, brain cancer, renal cancer, colon cancer, colorectal cancer, pancreatic cancer, bladder cancer, esophageal cancer, stomach cancer, genitourinary cancer, gastrointestinal cancer, uterine cancer, ovarian cancer, astrocytomas, gliomas, skin cancer, squamous cell carcinoma, Keratoakantoma, Bowen disease, cutaneous T-Cell Lymphoma, melanoma, basal cell carcinoma, actinic keratosis, sarcomas, Kaposi's sarcoma, osteosarcoma, head and neck cancer, small cell lung carcinoma, non-small cell lung carcinoma, leukemias, lymphomas, or other blood cell cancers, ichtiosis, acne, acne vulgaris, thyroid resistance syndrome, diabetes, thalassemia, cirrhosis, protozoal infection, rheumatoid arthritis, rheumatoid spondylitis, all forms of rheumatism, osteoarthritis, gouty arthritis, multiple sclerosis, insulin dependent diabetes mellitus, non-insulin dependent diabetes, asthma, rhinitis, uveithis, lupus erythematoidis, ulcerative colitis, Morbus Crohn, inflammatory bowel disease, chronic diarrhea, psoriasis, atopic dermatitis, bone disease, fibroproliferative disorders, atherosclerosis, aplastic anemia, DiGeorge syndrome, Graves' disease, epilepsia, status epilepticus, alzheimer's disease, depression, schizophrenia, schizoaffective disorder, mania, stroke, mood-incongruent psychotic symptoms, bipolar disorder, affective disorders, meningitis, muscular dystrophy, multiple sclerosis, agitation, cardiac hypertrophy, heart failure, reperfusion injury, and obesity.

19. A method according to claim 1, in which a prognostic conclusion can be made about the subject's disease.

20. A method according to claim 1, further comprising:
monitoring of the clinical effectiveness of the treatment; and
making a prognostic conclusion about the subject's response to a therapeutic treatment based at least in part on said monitoring.

21. A method for identifying compounds which modulate the expression or activity of any of the polynucleotides or expression products thereof as defined in claim 1, comprising
(a) contacting a candidate compound with cells which express said at least one polynucleotide or a polypeptide encoded thereby, or with cell membranes comprising said polypeptide, or respond to said polypeptide; and
(b) determining the effect of said candidate compound on the expression, activity, cellular localization or structural condition of said polynucleotide or polypeptide;
or determining a functional response of said cells.

22. A method according to claim 21, wherein said determining the effect comprises comparing said expression, activity, cellular localization or structural condition of said polynucleotide or polypeptide with the expression, activity, cellular localization or structural condition of said polynucleotide or polypeptide in cells which were not contacted with the candidate compound.

23. A method according to claim 22, further comprising:
selecting the candidate compound when the expression of said at least one polynucleotide or polypeptide in the cells which were contacted with the candidate compound is lower than in the cells which were not contacted with the candidate compound.

24. A method according to claim 21, further comprising comparing the viability of cells which were contacted with the candidate compound and the viability of cells which were not contacted with the candidate compound.

25. A compound which antagonizes or agonizes any one of the polynucleotides or expression products thereof as defined in claim 1, wherein said compound is identified by a method comprising:
(a) contacting a candidate compound with cells which express said polynucleotide or expression product thereof, or with cells membrane comprising said expression product thereof, or respond to said expression product thereof; and
(b) determining the effect of said candidate compound on the expression, activity, cellular localization, or structural condition of said polynucleotide or expression product thereof; or determining a functional response of said cells.

26. A compound according to claim 25 which is an antisense nucleic acid capable of suppressing the expression of a polynucleotides or expression products thereof, wherein said polynucleotide is selected from the group consisting of:
(i) a polynucleotide having a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:8, SEQ ID NO:9, and the corresponding RNA sequences,
(ii) a polynucleotide having a sequence complementary to any one of the sequence under (i),
(iii) a polynucleotide variant of any one of the polynucleotide sequences under (i) or (ii), and
(iv) combinations thereof.

27. A solid support on which at least one isolated polynucleotide is immobilized, wherein said isolated polynucleotide is selected from the group consisting of:
(i) a poly nucleotide having a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and fragments thereof,
(ii) a polynucleotide having a sequence complementary to any one of the sequences under (i),
(iii) a polynucleotide having a sequence which is an allelic variant of any one of the sequences under (i) or (ii), and
(iv) combinations thereof.

28. A solid support according to claim 27, wherein at least 9 different isolated polynucleotides are immobilized on said solid support, and said 9 different isolated polynucleotides have the nucleotide sequences as shown in SEQ ID NO:1, SEQ ID NO:2, SEQ ID NO:3, SEQ ID NO:4, SEQ ID NO:5, SEQ ID NO:6, SEQ ID NO:7, SEQ ID NO:8 and SEQ ID NO:9, respectively; or the corresponding complementary sequences.

29. A solid support according to claim 28, wherein at least 89 different isolated polynucleotides are immobilized on said solid support, and said 89 isolated polynucleotides have the nucleotide sequences in FIG. 1.

30. A method of treating, preventing, or suppressing a disease associated with increased activity or expression of a polynucleotide or polypeptide as defined in claim 1, comprising administering to a subject in need thereof
A) a polynucleotide, or expression product thereof, selected from the group consisting of:
(i) a polynucleotide having a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and the corresponding RNA sequences,
(ii) a polynucleotide having a sequence complementary to any one of the sequences under (i),
(iii) a polynucleotide variant of any one of the polynucleotides under (i) or (ii), and
(iv) combinations thereof, and/or
B) a compound identified by a method comprising:
(i) contacting a candidate compound with cells which express said polynucleotide or expression product thereof, or with cell membranes comprising said expression product thereof, or respond to said expression product thereof; and
(ii) determining the effect of said candidate compound on the expression, activity, cellular localization, or structural condition of said polynucleotide or expression product thereof; or determining a functional response of said cells.

31. The method according to claims 30, wherein said disease is selected from the group consisting of estrogen receptor-dependent breast cancer, estrogen receptor-independent breast cancer, hormone receptor-dependent prostate cancer, hormone receptor-independent prostate cancer, brain cancer, renal cancer, colon cancer, colorectal cancer, pancreatic cancer, bladder cancer, esophageal cancer, stomach cancer, genitourinary cancer, gastrointestinal cancer, uterine cancer, ovarian cancer, astrocytomas, gliomas, skin cancer, squamous cell carcinoma, Keratoakantoma, Bowen disease, cutaneous T-Cell Lymphoma, melanoma, basal cell carcinoma, actinic keratosis, sarcomas, Kaposi's sarcoma, osteosarcoma, head and neck cancer, small cell lung carcinoma, non-small cell lung carcinoma, leukemias, lymphomas, or other blood cell cancers, ichtiosis, acne, acne vulgaris, thyroid resistance syndrome, diabetes, thalassemia, cirrhosis, protozoal infection, rheumatoid arthritis, rheumatoid spondylitis, all forms of rheumatism, osteoarthritis, gouty arthritis, multiple sclerosis, insulin dependent diabetes mellitus, non-insulin dependent diabetes, asthma, rhinitis, uveithis, lupus erythematoidis, ulcerative colitis, Morbus Crohn, inflammatory bowel disease, chronic diarrhea, psoriasis, atopic dermatitis, bone disease, fibroproliferative disorders, atherosclerosis, aplastic anemia, DiGeorge syndrome, Graves' disease, epilepsia, status epilepticus, alzheimer's disease, depression, schizophrenia, schizoaffective disorder, mania, stroke, mood-incongruent psychotic symptoms, bipolar disorder, affective disorders, meningitis, muscular dystrophy, multiple sclerosis, agitation, cardiac hypertrophy, heart failure, reperfusion injury and obesity.

32. The method according to claim 30, wherein said method is selected from the group consisting of:
(a) administering to a subject a therapeutically effective amount of a compound which causes a decrease in the expression of said polynucleotide;
(b) administering to the subject a therapeutically effective amount of an antagonist to said polypeptide;
(c) administering to the subject a therapeutically effective amount of an agonist to said polypeptide;
(d) administering to the subject a nucleic acid molecule that inhibits the expression of the nucleotide sequence encoding said polypeptide;
(e) administering to the subject a polynucleotide as defined in claim 1; or a nucleotide sequence complementary to said nucleotide sequence in a form so as to effect production of said thereof encoded polypeptide activity in vivo;
(f) administering to the subject a therapeutically effective amount of a polypeptide that competes with said polypeptide for its ligand, substrate, or receptor;
(g) administering to the subject a therapeutically effective amount of an antibody directed against said polypeptide, and
(h) combinations thereof.

33. The method according to claim 30 characterized in that the progression of the subject's disease to metastatic tumor progression is suppressed by said method.

34. The method according to claim 30, wherein said disease is a minimal residual tumor disease.


SEQUENCE LISTING (18 sequences)

SEQ ID NO: 1 - DNA, 2428 bp, Homo sapiens
cgtccagtttgagtctaggttggagttggaaccgtggagatgcggaaggaaaccccaccccccctagtgcccccggcggcccgggagtggaatcttcccccaaatgcgcccgcctgcatggaacggcagttggaggctgcgcggtaccggtccgatggggcgcttctcctcggggcctccagcctgagtgggcgctgctgggccggctccctctggctttttaaggacccctgtgccgcccccaacgaaggcttctgctccgccggagtccaaacggaggctggagtggctgacctcacttgggttggggagagaggtattctagtggcctccgattcaggtgctgttgaattgtgggaactagatgagaatgagacacttattgtcagcaagttctgcaagtatgagcatgatgacattgtgtctacagtcagtgtcttgagctctggcacacaagctgtcagtggtagcaaagacatctgcatcaaggtttgggaccttgctcagcaggtggtactgagttcataccgagctcatgctgctcaggtcacttgtgttgctgcctctcctcacaaggactctgtgtttctttcatgcagcgaggacaatagaattttactctgggatacccgctgtcccaagccagcatcacagattggctgcagtgcgcctggctaccttcctacctcgctggcttggcatcctcagcaaagtgaagtctttgtctttggtgatgagaatgggacagtctcccttgtggacaccaagagtacaagctgtgtcctgagctcagctgtacactcccagtgtgtcactgggctggtgttctccccacacagtgttcccttcctggcctctctcagtgaagactgctcacttgctgtgctggactcaagcctttctgagttgtttagaagccaagcccacagagactttgtgagagatgcgacttggtccccgctcaatcactccctgcttaccacagtgggctgggaccatcaggtcgtccaccacgttgtgcccacagaacctctcccagcccctggacctgcaagtgttactgagtagattggatttaagacaaaaagcaagtcccccatgagtgtccacttctttgccctgccctctcagcttgtgagacaacacaggagccttctatagtatgttgatatgctagatctgtgccgttaataggcatcgtctctcagcctgagggaggctggattctgggttcctgtagtcacagggaggaaaagctttcttaaaaatggacatgtatgtgcgtgtgagtgtgtgtgtagatttatagtttttggtagtggcaggaataaaaaaaatccatcctacatcttccctaagcactgcctctctctcaccccccaaaacaagttgacgaaagggttttatgtagctgtctatgaggaattggccgtgtctgggtgggttatgggatgtgggcatccctgggttcttggaagcagctcttatgctactcatagagatgggattgactttatttttttatagtgcttaattcaccattatgagaaatgcttccagtcacaaaaatgcagcccagctcactctgaggaagaagcaggacttggtacggttttacacaactccttaccattaaactgaatcagaaatccattttctggctgaataaaaagtttggcttgcctgtgtaatgcccactcccttccccctggctccctagtgatgggacatatatgagagagaagtgtttttctatcatagacaccataggggaaagtttggggatgaaggagagcttaaaggtgtttcaattaagttagaaaactgacacaggctgttgagaattctttgccacttttcccaccccaaaacagcatggggcctgacatcttctgccctggtcccctttctcttgatgtggaaagtctgaatgcagtatttatagacttctaaggttttaaaatccagtatcaagaagaaaatcagaaatactggttggtgaaataaagagtttaggcattgttggcctgtcttttttgaagcatgtgtgttatgtgtagttagatatatttcacttatgtgagtcatcatggtgttggtcttgtagcccattatttttcctgtgcttccccagcttcccaaagtagctagttagaacttaaggtaaatatttattcttgggttggtggagtggatattgccagttaggagtcatggatcaattactgattatattgaaagtaaatataatcaattatgtacttttgagctttgcaggttcaatttaggtaaaaatcacattatgaaactgggaaagtctgaaggaatatgggcaaaatatttctcagtaaagcttccatgcttcacccttgacatgattacccttgagtaaaacatgggaatttgtaaaaaaaaaaaaaaaaaaaaa

SEQ ID NO: 2 - DNA, 2219 bp, Homo sapiens
ggcaggtgttgaggggctcccggtccggctgccgccgctcccccgctccggacccggggctccccctagcgccgctgaggagccgcctctgcggctccaggagggcgcaggagcgggactgagagcgcctggaggctcgagcggagggtaattcatttgcacacctgttagcaagaaacagaagttgaaggactggaacaagtgaactaggaaagagggaacgccaatccaaggatagaaggacaaggacagaatcaccagcactggctgaaggcctcctgtttcctgcgctttctccttttcctgtgaaatctccgaggagaagaaagaatgatggacagtttatcctttcactgccacaaggcctgtttacttggcagtaggtccttaagttccttgcttttttgctgctgtttggtgactggaagaggcaccagagactctcactctggggaggtttgctggcatgggtaatctcattaaggtgctaaccagggacatagaccacaatgcagcacattttttcttggactttgaaagtaccttaacatggggaatcttcttaaagttttgacatgcacagaccttgagcaggggccaaattttttccttgattttgaaaatgcccagcctacagagtctgagaaggaaatttataatcaggtgaatgtagtattaaaagatgcagaaggcatcttggaggacttgcagtcatacagaggagctggccacgaaatacgagaggcaatccagcatccagcagatgagaagttgcaagagaaggcatggggtgcagttgttccactagtaggcaaattaaagaaattttacgaattttctcagaggttagaagcagcattaagaggtcttctgggagccttaacaagtaccccatattctcccacccagcatctagagcgagagcaggctcttgctaaacagtttgcagaaattcttcatttcacactccggtttgatgaactcaagatgacaaatcctgccatacagaatgatttcagctattatagaagaacattgagtcgtatgaggattaacaatgtaccggcagaaggagaaaatgaagtaaataatgaattggcaaatcgaatgtctttgttttatgctgaggcaactccaatgctgaaaaccttgagtgatgccacaacaaaatttgtatcagagaataaaaatttaccaatagaaaataccacagattgtttaagcacaatggctagtgtatgcagagtcatgctggaaacaccggaatacagaagcagatttacaaatgaagagacagtgtcattctgcttgagggtaatggtgggtgtcataatactctatgaccacgtacatccagtgggagcatttgctaaaacttccaaaattgatatgaaaggttgtatcaaagttcttaaggaccaacctcctaatagtgtggaaggtcttctaaatgctctcaggtacacaacaaaacatttgaatgatgagactacctccaagcaaattaaatccatgctgcaataacaattctggaataagcacctgctgtagacagaagacagtattctgcaatgactgagaatgcagttttttagtgattgcaattactatctcatttattcttgcttttatttctttcctctgttcctcttccctcttttttaatcatgttcttaagacttcttttctgtgccaaaatcagtaaagttacactctgaagggatatcatcctttcaaacgggccatctaaggcagctaattatgcattgcattggggtctctactgagaaaaattctgtgacttgaactaaatatttttaaatgtggattttttttgaaactaatatttaatattgcttctcctgcatggcaaaactgcctattctgctatttaaaaaccctcaatgactttattttctactgccgcctttttcatgtgcaaccaaaatgaaaatgtttaaattaactgtgttgtacaaatggtacccaacacaaactttttttaaattagtaatacttttgtttaaagttttaagtttgcattttgactttttttgtaaggatgtatgttgtgtgtttaacctttattaactaacgttaaaagctgtgatgtgtgcgtagaatattacgtatgcatgttcatgtctaaagaatggctgttgatgataaaataaaaatcagctttcatttttctaaaaaaaaaaaaaaaaaaaaaaaaa

SEQ ID NO: 3 - DNA, 3816 bp, Homo sapiens
ggggtcgcgccgagccgagccgagccgagcggagccggcggagcctctggaatcacccgggtcgctgttcctgaggtggtcaaggtggacagggggcggtggtgatggcgcagtttgacactgaataccagcgcctagaggcctcctatagtgattcacccccaggggaggaggacctgttggtgcacgtcgccgaggggagcaagtcaccttggcaccgtattgaaaaccttgacctcttcttctctcgagtttataatctgcaccagaagaatggcttcacatgtatgctcatcggggagatctttgagctcatgcagttcctctttgtggttgccttcactaccttcctggtcagctgcgtggactatgacatcctatttgccaacaagatggtgaaccacagtcttcaccctactgaacccgtcaaggtcactctgccagacgcctttttgcctgctcaagtctgtagtgccaggattcaggaaaatggctcccttatcaccatcctggtcattgctggtgtcttctggatccaccggcttatcaagttcatctataacatttgctgctactgggagatccactccttctacctgcacgctctgcgcatccctatgtctgcccttccgtattgcacgtggcaagaagtgcaggcccggatcgtgcagacgcagaaggagcaccagatctgcatccacaaacgtgagctgacagaactggacatctaccaccgcatcctccgtttccagaactacatggtggcactggttaacaaatccctcctgcctctgcgcttccgcctgcctggcctcggggaagctgtcttcttcacccgtggtctcaagtacaactttgagctgatcctcttctggggacctggctctctgtttctcaatgaatggagcctcaaggccgagtacaaacgtggggggcaacggctagagctggcccagcgcctcagcaaccgcatcctgtggattggcatcgctaacttcctgccgtgccccctcatcctcatatggcaaatcctctatgccttcttcagctatgctgaggtgctgaagcgggagccgggggccctgggagcacgctgctggtcactctatggccgctgctacctccgccacttcaacgagctggagcacgagctgcagtcccgcctcaaccgtggctacaagcccgcctccaagtacatgaattgcttcttgtcacctcttttgacactgctggccaagaatggagccttcttcgctggctccatcctggctgtgcttattgccctcaccatttatgacgaagatgtgttggctgtggaacatgtgctgaccaccgtcacactcctgggggtcaccgtgaccgtgtgcaggtcctttatcccggaccagcacatggtgttctgccctgagcagctgctccgcgtgatcctcgctcacatccactacatgcctgaccactggcagggtaatgcccaccgctcgcagacccgggacgagtttgcccagctcttccagtacaaggcagtgttcattttggaagagttgttgagccccattgtcacacccctcatcctcatcttctgcctgcgcccacgggccctggagattatagacttcttccgaaacttcaccgtggaggtcgttggtgtgggagatacctgctcctttgctcagatggatgttcgccagcatggtcatccccagtggctatctgctgggcagacagaggcctcagtgtaccagcaagctgaggatggaaagacagagttgtcactcatgcactttgccatcaccaaccctggctggcagccaccacgtgagagcacagccttcctaggcttcctcaaggagcaggttcagcgggatggagcagctgctagcctcgcccaagggggtctgctccctgaaaatgccctctttacgtctatccagtccttacaatctgagtctgagcccctgagccttatcgcaaatgtggtagctggctcatcctgccggggccctccactgcccagagacctgcagggctccaggcacagggctgaagtcgcctctgccctgcgctccttctccccgctgcaacccgggcaggcgcccacaggccgggctcacagcaccatgacaggctctggggtggatgccaggacagccagctccgggagcagcgtgtgggaaggacagctgcagagcctggtgctgtcagaatatgcatccacagagatgagcctgcatgccctctatatgcaccagctccacaagcagcaggcccaggctgaacctgagcggcatgtatggcaccgccgggagagtgatgagagtggagaaagcgcccctgatgaagggggagagggcgcccgggccccccagtctatccctcgctctgctagctatccctgtgtagcaccccggcctggagctcctgagaccaccgccctgcatgggggcttccagaggcgctacggtggcatcacagatcctggcacagtgcccagggttccctctcatttctctcggctgcctcttggagggtgggcagaagatgggcagtcggcatcaaggcaccctgagcccgtgcccgaagagggctcggaggatgagctaccccctcaggtgcacaaggtatagacaaggctgagcagggttcctgtggcccaggatggaggccaccgctgccctgccatcccgtctgcctgccatgggacggctcctctgagtgttccctggccccatgtgtgtggtgtttgtgtgtctgtgcctggccaagggaggtgccaacactgggcttgccacagccccaggagaggaatttggggcctaggaaccgagggcacacgggactctagcctcatccccaggacccccttggctcagagtgtggtgctagaaactggtccccagcccagccccagtactgccacctttacacctacccctgcaagtccccagagggctgcccacgatagaagctgccaagcagggagaacctgtgccaactgtggagtggggaggttgggcctggaccctcaacccctgcaaccttccctagccccctcaatagatgagcaggtcaggctgtggcccttacctcacccgcagttctcgcccagtgctgcagccggctcacctctctccgcttcttgcacatcactggcctgtgtgtgctgcttgctcctgttctgttcgcttgctcccgttccgttcggcttttgctttgcgttagggtgaagaccctagcgtccagctcccctcaacgctatattttgacactaaaaaagaaggtttctaaattgtaggagcaggatggaaatactttgctgcccttgccatcttttaggatgggcccccaggagactgaggtcttcctgggccctcattgctgcttatcgtaccccccatcacctgcacatgggacagaccgggctggagggtgaccttggctgtgtacgtcccagcaaaagagctctggcccgcatctcgctgtgccctgaagggggatgaagggcgatgcctcgcccgaggctttgggctgctgcactgcatgctgggactgctcctactctctgtcccacccctcacccagctgtggtccggctttgggagagtggtgaattgcgctgcccgaactcggagcggagcagggtagggaccgtgtacagcttgataacccttaataaaaagggagtttgaccagaaaaaaaaaaaaaaaaaaaaaaaaaaaaaaaaaaaaaaaaaaaaaaaaaaaaaaaaaaaaaaaaaagaaaaaaaaaaaaaaaaaaagaaaaaaaaaaaaaaaaagaaaaaaaaaaaaaaaaaacct

SEQ ID NO: 4 - DNA, 4280 bp, Homo sapiens
gaattcaagaaatgttgccctggttcacctgatattgacaaactggacgttgccacaatgacagagtatttaaattttgagaagagtagttcagtctctcgatatggagcctctcaagttgaagatatggggaatataattttagcaatgatttcagagccttataatcacaggttttcagatccagagagagtgaattacaagtttgaaagtggaacttgcagcaagatggaacttattgatgataacaccgtagtcagggcacgaggtttaccatggcagtcttcagatcaagatattgcaagattcttcaaaggactcaatattgccaagggaggtgcagcactttgtctgaatgctcagggtcgaaggaacggagaagctctggttaggtttgtaagtgaggagcaccgagacctagcactacagaggcacaaacatcacatggggacccggtatattgaggtttacaaagcaacaggtgaagatttccttaaaattgctggtggtacttccaatgaggtagcccagtttctctccaaggaaaatcaagtcattgttcgcatgcgggggctccctttcacggccacagctgaagaagtggtggccttctttggacagcattgccctattactgggggaaaggaaggcatcctctttgtcacctacccagatggtaggccaacaggggacgcttttgtcctctttgcctgtgaggaatatgcacagaatgcgttgaggaagcataaagacttgttgggtaaaagatacattgaactcttcaggagcacagcagctgaagttcagcaggtgctgaatcgattctcctcggcccctctcattccacttccaacccctcccattattccagtactacctcagcaatttgtgccccctacaaatgttagagactgtatacgccttcgaggtcttccctatgcagccacaattgaggacatcctggatttcctgggggagttcgccacagatattcgtactcatggggttcacatggttttgaatcaccagggccgcccatcaggagatgcctttatccagatgaagtctgcggacagagcatttatggctgcacagaagtgtcataaaaaaaaacatgaaggacagatatgttgaagtctttcagtgttcagctgaggagatgaactttgtgttaatggggggcactttaaatcgaaatggcttatccccaccgccatgtaagttaccatgcctgtctcctccctcctacacatttccagctcctgctgcagttattcctacagaagctgccatttaccagccctctgtgattttgaatccacgagcactgcagccctccacagcgtactacccagcaggcactcagctcttcatgaactacacagcgtactatcccagccccccaggttcgcctaatagtcttggctacttccctacagctgctaatcttagcggtgtccctccacagcctggcacggtggtcagaatgcagggcctggcctacaatactggagttaaggaaattcttaacttcttccaaggttaccagtgtttgaaagatgtatggtgatcttgaaacctccagacacaagaaaacttctagcaaattcaggggaagtttgtctacactcaggctgcagtattttcagcaaacttgattggacaaacgggcctgtgccttatcttttggtggagtgaaaaagtttgagctagtgaagccaaatcgtaacttacagcaagcagcatgcagcatacctggctctttgctgattgcaaataggcatttaaaatgtgaatttggaatcagatgtctccattacttccagttaaagtggcatcataggtgtttcctaagttttaagtcttggataaaaactccaccagtgtctaccatctccaccatgaactctgttaaggaagcttcatttttgtatattcccgctcttttctcttcatttccctgtcttctgcataatcatgccttcttgctaagtaattcaagcataagatcttggaataataaaatcacaatcttaggagaaagaataaaattgttattttcccagtctcttggccatgatgatatcttatgattaaaaacaaattaaattttaaaacacctgaaaaaaaaaaaaaaaaagaattcaagaaatgttgccctggttcacctgatattgacaaactggacgttgccacaatgacagagtatttaaattttgagaagagtagttcagtctctcgatatggagcctctcaagttgaagatatggggaatataattttagcaatgatttcagagccttataatcacaggttttcagatccagagagagtgaattacaagtttgaaagtggaacttgcagcaagatggaacttattgatgataacaccgtagtcagggcacgaggtttaccatggcagtcttcagatcaagatattgcaagattcttcaaaggactcaatattgccaagggaggtgcagcactttgtctgaatgctcagggtcgaaggaacggagaagctctggttaggtttgtaagtgaggagcaccgagacctagcactacagaggcacaaacatcacatggggacccggtatattgaggtttacaaagcaacaggtgaagatttccttaaaattgctggtggtacttccaatgaggtagcccagtttctctccaaggaaaatcaagtcattgttcgcatgcgggggctccctttcacggccacagctgaagaagtggtggccttctttggacagcattgccctattactgggggaaaggaaggcatcctctttgtcacctacccagatggtaggccaacaggggacgcttttgtcctctttgcctgtgaggaatatgcacagaatgcgttgaggaagcataaagacttgttgggtaaaagatacattgaactcttcaggagcacagcagctgaagttcagcaggtgctgaatcgattctcctcggcccctctcattccacttccaacccctcccattattccagtactacctcagcaatttgtgccccctacaaatgttagagactgtatacgccttcgaggtcttccctatgcagccacaattgaggacatcctggatttcctgggggagttcgccacagatattcgtactcatggggttcacatggttttgaatcaccagggccgcccatcaggagatgcctttatccagatgaagtctgcggacagagcatttatggctgcacagaagtgtcataaaaaaaaacatgaaggacagatatgttgaagtctttcagtgttcagctgaggagatgaactttgtgttaatggggggcactttaaatcgaaatggcttatccccaccgccatgtaagttaccatgcctgtctcctccctcctacacatttccagctcctgctgcagttattcctacagaagctgccatttaccagccctctgtgattttgaatccacgagcactgcagccctccacagcgtactacccagcaggcactcagctcttcatgaactacacagcgtactatcccagccccccaggttcgcctaatagtcttggctacttccctacagctgctaatcttagcggtgtccctccacagcctggcacggtggtcagaatgcagggcctggcctacaatactggagttaaggaaattcttaacttcttccaaggttaccagtgtttgaaagatgtatggtgatcttgaaacctccagacacaagaaaacttctagcaaattcaggggaagtttgtctacactcaggctgcagtattttcagcaaacttgattggacaaacgggcctgtgccttatcttttggtggagtgaaaaagtttgagctagtgaagccaaatcgtaacttacagcaagcagcatgcagcatacctggctctttgctgattgcaaataggcatttaaaatgtgaatttggaatcagatgtctccattacttccagttaaagtggcatcataggtgtttcctaagttttaagtcttggataaaaactccaccagtgtctaccatctccaccatgaactctgttaaggaagcttcatttttgtatattcccgctcttttctcttcatttccctgtcttctgcataatcatgccttcttgctaagtaattcaagcataagatcttggaataataaaatcacaatcttaggagaaagaataaaattgttattttcccagtctcttggccatgatgatatcttatgattaaaaacaaattaaattttaaaacacctgaaaaaaaaaaaaaaaaa

SEQ ID NO: 5 - DNA, 1384 bp, Homo sapiens
accgttcttttaactgcgcaggcgcgccggaagcacctagagagcggcgcgtgcgcagcgggagtcgaagcggagatcccggggtcgcgcgagagccgcaagcggagttggtgggcgctatgctatcacccgaggcagagcgagtgctgcggtaccttgtagaagtggaggagctcgccgaggaggtgctggcggacaagcggcagattgtggacctggacactaaaaggaatcagaatcgagagggcctgagggccctgcagaaggatctcagcctctctgaagatgtgatggtttgcttcgggaacatgtttatcaagatgcctcaccctgagacaaaggaaatgattgaaaaagatcaagatcatctggataaagaaatagaaaaactgcggaagcaacttaaagtgaaggtcaaccgcctttttgaggcccaaggcaaaccggagctgaagggttttaacttgaaccccctcaaccaggatgagcttaaagctctcaaggtcatcttgaaaggatgagactcaagaaccaagatgggggaccagcaaccccccagggtcatggaggacccaggaccctccaaccttgacacctgtaaggacaggatctgccctgtaaggggccagccgtcaggaatctggccatgaaaacctctttgtagtgcttggctactctgtgatggcaggagggaaccttcagcctgtctggctgctggacctggacaccagggctcggtggacacaagatctattgacgggccttggtagccaccagtgggtgtgtggggcagtggctgtgggggtgtaagaatgactgcaacaggcacttcccaacaatggcctgctgttcacatggaccctgagcaaggaaggagggagggaggggcagagtggagtgtcattccagcattcctctcagaagggagagaggttttcaggctggtgccatgcgattggaataaagcaggaggctcatgggtggttgctgaatgaagaacagaatcttggtgctttgtggctcaccacagccatctgtggggcaggcacacacacctcccgccagctccaattttgcactttttccctgcttgattccaagagtaggtgctgcctagcagcccttcgtggccactctttactcaggagggccttgcagagtcctgcaccaggcctgggtgagtggatgcgcctcttaccatatgacacgtgtcaagatgcccttccgccccctctgaaagtggggcccggccagcactgctcgttactgtctgccttcagtggtctgaggtcccagtatgaactgccgtgaagtcaaaactcttatgtgttcattaagggctcaataaatgttagctgaatgaatgaatagcaaaaaaaaaaaa

SEQ ID NO: 6 - DNA, 4159 bp, Homo sapiens
gtgcgcccttgcttcgtgccctcaacccgcatggcggagccgctggcgcgccgcggagaggccgggcgagtcgggcggtttcggcgcccgcgctgagccgcggaggaggggcggaggacgcccctgcagccggtgcgtctgccctcagtgaggcggggcgcgcggcggacgcccccgggcaggggcgggagtggtggaggcgccggcggttggcactgacaggggcggtgagcgagccgctccggtctccgggcgaggcttggccttccgagcagagacggcgggaagcggcggcggcagcggcggccctagggccggctggtgaggcgatggcggcgccggccccgggggctggggcagcctcgggcggcgctggctgtagcggcggcggcgcgggcgcgggcgcgggctcgggctctggggccgcgggggccgggggccggctgcccagccgggtgctggagttggtgttctcttacctggagctgtccgagctgcggagctgcgccctggtgtgcaagcactggtaccgctgcctgcacggcgatgagaacagcgaggtgtggcggagcctgtgcgcccgcagcctggcagaagaggctctgcgcacggacatcctgtgcaacctgcccagctacaaggccaagatacgtgcttttcaacatgccttcagcactaatgactgctccaggaatgtctacattaagaagaatggctttactttacatcgaaaccccattgctcagagcactgatggtgcaaggaccaagattggtttcagtgagggccgccatgcatgggaagtgtggtgggagggccctctgggcactgtggcagtgattggaattgccacaaaacgggcccccatgcagtgccaaggttatgtggcattgctgggcagtgatgaccagagctggggctggaatctggtggacaataatctactacataatggagaagtcaatggcagttttccacagtgcaacaacgcaccaaaatatcagataggagaaagaattcgagtcatcttggacatggaagataagactttagcttttgaacgtggatatgagttcctgggggttgcttttagaggacttccaaaggtctgcttatacccagcagtttctgctgtatatggcaacacagaagtgactttggtttaccttggaaaacctttggacggatgacagtggctttcttgtgatgacagacagaatggaggagagatctgcttatgggaagtagaaccatgaagtgactgtcacacatgcatgtccaagaaacatcctgaaaacacatgaagtcgtaaactggagaagcagctctacagcagagattatcttcgtgtttcctctttctactgggccagaaaaatcctcagggttgcagttggttgagtgggcagttgacatatgcatgttgcacccgatgttgtctctaagttagcaatgtgttatttccagctttaaaggtgagattgtagagatgctgtcaaagggataaggaaatagcaagatttttaagtagtgtgtttgtgaagactgatcccattttacaactgcctgttctttctccagtccttttttttccagccagcttgactattagaaaagtatgaaactggttgggttttatttaatatttttaatatattgagaagcatggtctgcctggactgcacttctctaaaagtgagatataaaattgtgcagctattttaaaagttgtatataatatgtgtgtaaaaaaaaaaaactgtaaaaaagaaaggacaaacaggttgttttgttctagttctaatttcttaaaaaccactacatggttacaaaattggaataacatttggggacaactgggttaactacaaagaagaggattttaagaggagatgtgttgtattgactcattttgtattatttttggcttacagttcccatagctgttagagtctggtttgtttttgtttttactctcaaaatcatagtaaagatctctcagtctcctggctaaagattgaaggaaggcaaatctatttctaattatacatatatcagtaaggatgatctcaacataatagtaatgtgtatcttttggtatccagttttatttttggccttctaagaaagtgtctcataacacagaacattgccatttgctcttgtaggcctcaaatatgaaagctattagtcatagagcctaggaaaaaaagaattgattaatggtccttttattttgtaaccttataaatgctgtagatattatcaaaaaaattttaatttcatattgtttacatcatgcaactaatctaagcctcaaactcgttattggggctataaagaaaacgtttacttacccagctgaaacaggttaagaatattcttaatctcattatagataattgcccccatgggacttgaaatacaacaccttgtgctgaaaacttcaggttggcaatatttgaaggtttcgttgtagaagagtttaacattaactcctattttgacttacaaatcttgtttctcatcactaaaatgcttttgaattaataatccaacccacatgagctgagagtttttcttttgttagaaaagaaacagacatctttctgtatgaaagtataaattgtatggttttagatacataagaattgacaaaagcgagcgaaatctttgtacttctgagttcttgctgtatgtatgttttgttttaaatctgattagggacacccagcagctggccgggattcttggattgctccttgggagttaagattgtcaatactcctgtgaagcaagggatttcagccatagaacaaagatttattgttgccacctgaaaagtttacaagtatttattgtgtatttgatacattgcttgaaaagatgaaatctgttaaagattcttttcgatgtccaggttaagaagaaacctccttgtattgagtgaaattatatgttaaatgtattagagaatgtaggtggtatagaaattgatttttcttggtgtagaacaactcagttcggcaaagtttaaaatttgattaaacaagagaagtggttcaggttgaagatggacttgttaggaagtgatcaagtcctttaagtacttgtttctttttcaggttgtgatgtggccattccgaattttgttgagagtttggtttataattgtctcttttgtcttgttagtaaacattcatttgcaacagttttgaaggtgctgagtggaaaaccgaaacacatggttattgcgtattggacctagaatgaaataattgcctcaatatttaacaacaagccattcttatctcaaagatttaaattcccgaatgtcccattcgcaaatcatatgcaattgaagtgagcagcatgagcatctgggtcatgagggccttcatttacgtaaatttgtcactaaaacccagtagtagctctacaaaatcttaaactgctgcagtgctcaaggagatggaatatctttgtcattggtgctgaggagagcatttcggtagaagacagttgcgcctgaagattgagtgtaaatcattcaaaccagtggttctcagtgttggctgtatacactttgtagtcactttggaatgttggaagacacatcgatgcttgggttccgtatgccaagattctgatgttggtctggaatatgagctggtcataaggatttttaaaaactttctggtcatttcaatatgctgccaaggttgagaaccactgttgtaaaattcaccttgagttttctcatctgcaaaatagaaaaaaaaaaatccttgctccctcccttcactacctcacaaggatattgagggtaaaggagaaaataatgggaaagtgcttgtgccgtggatgaaaagtgctattaaaagtcaaaggagtgttctgtttcaattcatagtatgatcagggaaagtgtaactgagtatactttgttgacttgggaaacctggagcactttctttggttggttaacgaagcatgcagatgtggaagcagacgttactattatccctactatggtcttctgtcatactgagacaggctgttttaattacctggttttacataggaaagaagaaatattaaggcttaaagtttgtaatgatcaatggctcataattcattaaatcttttcatacaaggaa

SEQ ID NO: 7 - DNA, 4021 bp, Homo sapiens
ggtagccgccccgccccgcggggcgccacgggcgggtcttggcagcgcccactgagccagccgggccgcaggtgccgcccccgatacacggtgtcccgcccaagctgatccgcgtctgcggtcggtcggtgcgtgcgtgcgcctcgtcggtccgcgtgtctggccgagagcccccttcctctgcggccatgactccgccgccgccgccgccccctcccccgggccctgaccccgcggccgaccccgccgcggacccctgcccctggcccggatcactggtcgtcctcttcggggctacggcgggtgcgctgggacgggacctgggctcggacgagaccgacttaatcctcctagtttggcaagtggttgagccgcggagccgccaggtggggacgctgcacaaatcgctggttcgtgccgaggcggccgcactgagtacgcagtgccgcgaggcgagcggcctgagcgccgacagcctggcgcgggcagagccgctggacaaggtgctgcagcagttctcacagctggtgaacggggatgtggctttgctgggcgggggcccctacatgctctgcactgatgggcagcagctattgcgacaggtcctgcaccccgaggcctccaggaagaacctggtgctccccgacatgttcttctccttctatgacctccgaagagaattccatatgcagcatccaagcacctgccctgccagggacctcactgtggccaccatggcacagggtttaggactggagacagatgccacagaggatgactttggggtctgggaagtcaagacaatggtagctgttatcctccatctactcaaagagcccagcagtcaattgttttcgaagcccgaggtgataaagcagaaatacgagacggggccttgcagcaaggctgatgtggtggacagtgagactgtggtacgggctcgtgggttgccgtggcagtcatcagaccaggacgtggctcgcttcttcaaagggctcaacgtggccaggggtggtgtagcactctgcctcaacgcccagggccgcagaaatggcgaggccctcatccgctttgtggacagcgagcagcgggacctagcgctgcagagacacaagcaccacatgggcgtccgctatattgaggtgtataaagcgacaggggaggagtttgtaaagattgcagggggcacatcactagaggtggctcgtttcttgtcacgggaagaccaagtgatcctgcggctgcggggactgcccttctcggctgggccaacggacgtgcttggcttcctggggccagagtgcccagtgactgggggtaccgaggggctgctctttgtgcgccatcctgatggccggccgactggtgatgccttcgccctctttgcttgtgaggagctggcacaggctgcactgcgcaggcacaagggcatgctgggtaagcgatacattgaactcttccggagcactgcagccgaagtgcagcaggtcttgaaccgctatgcatccggcccactccttcctacactgactgccccactgctgcccatccccttcccactggcacctgggactgggagggactgtgtacgcctccgaggcctgccctacacggccaccattgaagacatcctgagctttctgggggaggcagcagctgacattcggccccacggtgtacacatggtgctcaaccagcagggccggccatcgggcgatgccttcattcagatgacatcagcagagcgagccctagctgctgctcagcgttgccataagaaggtgatgaaggagcgctacgtggaggtggtcccctgttccacagaggagatgagccgagtgctgatggggggcaccttgggccgcagtggcatgtcccctccaccctgcaagctgccctgcctctcaccacctacctacaccaccttccaagccaccccaacgctcattcccacggagacggcagctctatacccctcttcagcactgctcccagctgccagggtgcctgctgcccccacccctgttgcctactatccagggccagccactcaactctacctgaactacacagcctactacccaagccccccagtctcccccaccactgtgggctacctcactacacccactgctgccctggcctctgctcccacctcagtgttgtcccagtcaggagccttggtccgcatgcagggtgtcccatacacggctggtatgaaggatctgctcagcgtcttccaggcctaccagctacccgctgatgactacaccagtctgatgcctgttggtgacccacctcgcactgtgttacaagcccccaaggaatgggtgtgtttgtaggagagaaagccaggaggtaagagccagctgatatcctcggcgaacatgtctctcctgagtccagaagaccagcaccctcaacctggtagcttctttctggcttgtcaaagctctcagaaggtacctagaggagcccaagccccagctccatcctccacttattctgcctgtttcccccaaagacaatggctggaccctgcatgcagggctgggggtggaatggggctaaccagctcctgatggcctgagccaggcatcttgactggcacctggagagcccttaagtctgtcctggctgtggcccatgccgacagatatcgtggggctgacaggtccacggcaggcttgctttcttttataaaatggaagctctggtaccttcaatgtatgactcctgggagaatcaagggtccatctgagcctctgagtaaagatcccaatgttctacctctccctgtccctcttgtaggggatagggaggcagagagagccagcccctaccctcagagtatctggacctcagagaccatgttgtgccaggggtggtcccacctaaagatgctagcccctctccaggtgggcataaggagtaacagatggcaaaaccacaaactattttgatggactgtgctgcagtatcaccagaagacattagggggcagtaggcccccacacaaaaccttcaggcttgaattttaaaggggaggactttctgccaacttttcttgtatgccttgggaaagccagttgccctgaacccagcagacaccatggaatgtcctttgcacgcattaaatggtacagaactgaagcctcggaagcaatttggaactcgatcttctcttccttaaatgaaaagttattgaccaaatggactttttaaaagacacaggacccttaactttgccccaaagtgaggggctccacaccaaccccaggcggaggaacactcagacagattaaggatactgttgacctgtcactgtttattatttcagcactaaaactgaggagcctcaactgctggctcttcttccctttgtatttgtgtaaggagcactgcactcccataaaaggttttaaaatacaaaatgtacaagaacacacaattccaagtgctgtaaacataactgagaaccagttcctttactaaacatccattttataaaacacaaggtttcaatttgagcccatctgagccttaaagatccattctgaataccaaaaacagggcttcacagccaggcccagaagaggtctggtgataatggctggccctgggtggggatagtttacacccgggcagcagcaccacacatgaacccaaagacatgttctttttaaagctgttttcagccatgtttctctgtgcatctccagtaagcagaaggctacccattccattcctcaacccaagagctagcacagttagagtaggagggggtgcgtactagcacgtgcccagttgctcagtgctgctagtagaaattgatttgcatagtccaatggatgtgtgctttaacaccactatgttgcacaaaaatttaagtctttatctacaaagccaaaaaatattgactcttaacaccaaagcttttacaaagctgatataaaactgcttacatagtatacaaagctctattttaaaatttaatgtttattttaaataggaaagcatt

SEQ ID NO: 8 - DNA, 1810 bp, Homo sapiens
gaaggccctgccgggcggcggcggcggcgacagcgtgcgagccatggtcgcgctggagaaccccgagtgcggcccggaggcggcggagggcaccccgggcgggcggcggctgctgccccttcccagctgcctgcctgccctagccagctcccaggtgaagagactctcggcttccaggcggaaacagcacttcatcaaccaggcagtgcggaactcagacctcgtgcccaaggccaaggggcggaagagcctccagcgcctggagaacacccagtacctcctgaccctgctggagacagacgggggcctgcctggcctggaggatggggacttggcaccccctgcatcaccaggcatctttgccgaggcctgcaacaacgccacctatgtggaggtctggaacgatttcatgaaccgctccggggaggagcaggagcgggttcttcgctacctggaggatgagggcaggagcaaggcgcggaggaggggccctggccgtggggaggaccggaggagagaggaccccgcctatacaccccgcgagtgcttccagcgcatcagccggcgtctgcgagccgtcctcaagcgcagccgcatccccatggaaacgctggagacctgggaggagcggctgcttcggttcttctccgtgtccccccaggccgtgtacacagcaatgctagacaacagcttcgagaggcttctgctgcacgctgtctgccagtacatggacctcatctcggccagtgctgacctggaggggaagcggcagatgaaggtcagtaatcggcacctggatttcctgccgccggggctgctcctgtccgcctacctggagcagcacagctgatggcggccccgcggagaccccgctgccacctcgcccagccatcaagccctccgataccttcggctaaaatatctttcatatttttagaatttgtcctcggaaacctttttcgcttggggtggtctctctcactctgccccctcctcacgcagctcttggcagtcaacagacgctggcggctggggctgcccatgccatcccagctccaagcttcccactccgggacttgtgtttgggtggggagacctgacctgggcatgttcctgtttcttcatcgttgagcttttctggcccggtctgaagctcaagtgaggagggggaggctgggtttttatcacttttaatgaatttggtgtgatttgttgtagatttttaaatttcccttttggagagaaaaaccaaaaaaactcgccccactggtaaaacatgggtcttggtcccagcccctgctcagcccctcccagtttttagcttgaatgagggtggggtctctgggaccctgcccctcatgccagaagcatcttgtgttgtatatgtgtgcgcgcgtgtgccctgagacccaggacagaagccacggtcctaagagccggttttatcctcgtcattctgcgtgtcctcccccacgccacctgtgtcggggctcagggtctcctgctttatatgagcccccttcctttcctcccctcctttatgctgggggtccaggacttccagccagaagcctctgcccttgcactaccttgtctgtcaccccatcccgtgtcccctcgtcccccagcctgactcctgcctgatagctcctgtgtccccatgctggtcctcctggcccaggctgcaggagccaggctggggggcctccgcacccccttgctgcgtgtgggtaattgtgttttgggggaaagtggggaatttaataaatttctggtgctctggcaaaaaaaaaaaaaaaaaaaaaaaaaa

SEQ ID NO: 9 - DNA, 3417 bp, Homo sapiens
cgaggctggcatagcggctgccgacccgccttcgttcctccaccccctgcacgggactgctgggcccgccccgccccgcctgcaggtgaagcggccgcagccgccgagtaggtgcgtggggatgatctcactcgcgcgctccgcgccaggaggaggaggagcgggagcggatccaacttccgggtagtggagccgcaagccaccggcatcttgctttttcttccccctcctcctgtgtgccccgcgccgctccctctttcccttttattcccggccccacccgccaaaatgaacagctcggacgaagagaagcagctgcagctcattaccagtctgaaggagcaagcaataggcgaatatgaagaccttagagcagagaaccagaaaacaaaggagaagtgtgacaaaattaggcaagaacgagatgaagccgttaaaaaactggaagaatttcagaaaatttctcacatggtcatagaggaagttaatttcatgcagaaccatcttgaaatagagaagacttgtcgagaaagtgctgaagctttggcaacaaagctaaataaagaaaataaaacgttgaaaagaatcagcatgttgtacatggccaagctgggaccagatgtaataactgaagagataaacattgatgatgaagattcgactacagacacagacggtgccgccgagacttgtgtctcagtacagtgtcagaagcaaattaaagaacttcgagatcaaattgtatctgttcaggaggaaaagaagattttagccattgagctggaaaatctcaagagcaaactcgtagaagtaattgaagaagtaaataaagttaaacaagaaaagactgttttaaattcagaagttcttgaacagagaaaagtcttagaaaaatgcaatagagtgtccatgttagctgtagaagagtatgaggagatgcaagtaaacctggagctggagaaggaccttcgaaagaaagcagagtcatttgcacaagagatgttcattgagcaaaacaagctaaagagacaaagccaccttctgctgcagagctccatccctgatcagcagcttttgaaagctttagacgaaaatgcaaaactcacccagcaacttgaagaagagagaattcagcatcaacaaaaggtcaaagaattagaagagcaactagaaaatgaaacactccacaaagaaatacacaacctcaaacagcaactggagcttctagaggaagataaaaaggaattggaattgaaatatcagaattctgaagagaaagccagaaatttaaagcactctgttgatgaactccagaaacgagtgaaccagtctgagaattcagtacctccaccacctcctcctccaccaccacttccccctccacctcccaatcctatccgatccctcatgtccatgatccggaaacgatcccaccccagtggcagtggtgctaagaaagaaaaggcaactcaaccagaaacaactgaagaagtcacagatctaaagaggcaagcagttgaagagatgatggatagaattaaaaagggagttcatcttagacccgttaatcagacagccagaccgaagacaaagccagaatcttcgaaaggctgcgaaagtgcagtggatgaactaaaaggaatactggcctcccagtagcattggatgcaggaaaaaatacattgacggtgaaaaacaagccgaaccagttgtagttttagatcctgtttctacacatgaaccccaaaccaaagaccaggttgctgaaaaagatccaactcaacacaaggaggatgaaggcgaaattcaaccagaaaacaaagaagacagcattgaaaacgtgagagagacagacagctccaactgctgatccataaaccagaagcctgatacgtttggaagtccttttcaataagcacatgattagtgttgttatattggcaagggctgtagacattctgctctggtcactgtattcagaatacaggttcttttctggtgtcacttttgtaagtagcaactataaacataagtaagctgtttagcaaaacacacattcctagtaggttttggttttttgatctttataaagatgaggtttttttcctagttactgtattaagtatgacttcttttagaaggttacaaaaaaattcagatgttgatacctttttaggaaatgtgcataccactcatcaaatggaatgctgaaagtttgaggtgcttgtatataatcggataaacaaaactgatcaacccaatgtgattttaaaagcccccaaagaagcttctgttttgggtctgatcctcttgatggagaaactgcagcagcatggaaattgttgggtactgtggcatacaagttattttctacagtagactgagataaactgaaaactcaggagctggcatcaaactcgtagtcccatagtcagtgttaattacacacattgttaactattggatgaaaaatacatgctattgattgtgtccaaagcctcccgaggacctccgtggggatgctctggtagcctgaatacagaactgaggtgaaagtccaaaccttgaattttacagtagtaagttggtaaaccatgtgctctgtgctatgagttaattatgttttcccaaatactaatgtggcacaagtaccatattttatcagagttcttatgtacagtatggtgaagataagtgacaagcacacatttttcttgcttcactgctgttctatattacacaggtttgttgttgttttttttaaaaaagaaattaagcagtagttagtctctaaaaatacaatgtttcaggctaccacagtgaataaatagaaatgtaatcagggattaaaaaaaaaacttatgcagcttttcaaagttgattgtttcaaaattggtgtttatttaaaataagtggtaatgtacttgaatgcactttttatgacaatgattcagtaatggtaattttactattaaagaaagtgaaaggtttagttttgttagcatggctcagcatgtagctgtcaggtgtttttcacctaagggcaaaagaaaatgatagtaataattgcagtagttgtattgtattgtatttttgcacgtgtggtaagcataggcttgaagaggtgggtaggcaggtacatgtacttcctaaattttgagataattatctttctgtaagttcgttatgcttgactgtttccatgttctcccaataatgattttatagttacttatcactttactcatggagaattaaaacgtaatgtttttcaactgtatctttctttaactggataatactgctatatgatatgcttactacagactgcattaattcacgaaacgaattctgttatgctgtaatttgaactctcctcaccacaacttattaaaaaggcaccaatagtttcccatt

SEQ ID NO: 10 - protein, 342 aa, Homo sapiens
MRKETPPPLVPPAAREWNLPPNAPACMERQLEAARYRSDGALLLGASSLSGRCWAGSLWLFKDPCAAPNEGFCSAGVQTEAGVADLTWVGERGILVASDSGAVELWELDENETLIVSKFCKYEHDDIVSTVSVLSSGTQAVSGSKDICIKVWDLAQQVVLSSYRAHAAQVTCVAASPHKDSVFLSCSEDNRILLWDTRCPKPASQIGCSAPGYLPTSLAWHPQQSEVFVFGDENGTVSLVDTKSTSCVLSSAVHSQCVTGLVFSPHSVPFLASLSEDCSLAVLDSSLSELFRSQAHRDFVRDATWSPLNHSLLTTVGWDHQVVHHVVPTEPLPAPGPASVTE

SEQ ID NO: 11 - protein, 324 aa, Homo sapiens
MGNLLKVLTCTDLEQGPNFFLDFENAQPTESEKEIYNQVNVVLKDAEGILEDLQSYRGAGHEIREAIQHPADEKLQEKAWGAVVPLVGKLKKFYEFSQRLEAALRGLLGALTSTPYSPTQHLEREQALAKQFAEILHFTLRFDELKMTNPAIQNDFSYYRRTLSRMRINNVPAEGENEVNNELANRMSLFYAEATPMLKTLSDATTKFVSENKNLPIENTTDCLSTMASVCRVMLETPEYRSRFTNEETVSFCLRVMVGVIILYDHVHPVGAFAKTSKIDMKGCIKVLKDQPPNSVEGLLNALRYTTKHLNDETTSKQIKSMLQ

SEQ ID NO: 12 - protein, 839 aa, Homo sapiens
MAQFDTEYQRLEASYSDSPPGEEDLLVHVAEGSKSPWHRIENLDLFFSRVYNLHQKNGFTCMLIGEIFELMQFLFVVAFTTFLVSCVDYDILFANKMVNHSLHPTEPVKVTLPDAFLPAQVCSARIQENGSLITILVIAGVFWIHRLIKFIYNICCYWEIHSFYLHALRIPMSALPYCTWQEVQARIVQTQKEHQICIHKRELTELDIYHRILRFQNYMVALVNKSLLPLRFRLPGLGEAVFFTRGLKYNFELILFWGPGSLFLNEWSLKAEYKRGGQRLELAQRLSNRILWIGIANFLPCPLILIWQILYAFFSYAEVLKREPGALGARCWSLYGRCYLRHFNELEHELQSRLNRGYKPASKYMNCFLSPLLTLLAKNGAFFAGSILAVLIALTIYDEDVLAVEHVLTTVTLLGVTVTVCRSFIPDQHMVFCPEQLLRVILAHIHYMPDHWQGNAHRSQTRDEFAQLFQYKAVFILEELLSPIVTPLILIFCLRPRALEIIDFFRNFTVEVVGVGDTCSFAQMDVRQHGHPQWLSAGQTEASVYQQAEDGKTELSLMHFAITNPGWQPPRESTAFLGFLKEQVQRDGAAASLAQGGLLPENALFTSIQSLQSESEPLSLIANVVAGSSCRGPPLPRDLQGSRHRAEVASALRSFSPLQPGQAPTGRAHSTMTGSGVDARTASSGSSVWEGQLQSLVLSEYASTEMSLHALYMHQLHKQQAQAEPERHVWHRRESDESGESAPDEGGEGARAPQSIPRSASYPCVAPRPGAPETTALHGGFQRRYGGITDPGTVPRVPSHFSRLPLGGWAEDGQSASRHPEPVPEEGSEDELPPQVHKV

SEQ ID NO: 13 - protein, 358 aa, Homo sapiens
MTEYLNFEKSSSVSRYGASQVEDMGNIILAMISEPYNHRFSDPERVNYKFESGTCSKMELIDDNTVVRARGLPWQSSDQDIARFFKGLNIAKGGAALCLNAQGRRNGEALVRFVSEEHRDLALQRHKHHMGTRYIEVYKATGEDFLKIAGGTSNEVAQFLSKENQVIVRMRGLPFTATAEEVVAFFGQHCPITGGKEGILFVTYPDGRPTGDAFVLFACEEYAQNALRKHKDLLGKRYIELFRSTAAEVQQVLNRFSSAPLIPLPTPPIIPVLPQQFVPPTNVRDCIRLRGLPYAATIEDILDFLGEFATDIRTHGVHMVLNHQGRPSGDAFIQMKSADRAFMAAQKCHKKKHEGQIC

SEQ ID NO: 14 - protein, 133 aa, Homo sapiens
MLSPEAERVLRYLVEVEELAEEVLADKRQIVDLDTKRNQNREGLRALQKDLSLSEDVMVCFGNMFIKMPHPETKEMIEKDQDHLDKEIEKLRKQLKVKVNRLFEAQGKPELKGFNLNPLNQDELKALKVILKG

SEQ ID NO: 15 - protein, 286 aa, Homo sapiens
MAAPAPGAGAASGGAGCSGGGAGAGAGSGSGAAGAGGRLPSRVLELVFSYLELSELRSCALVCKHWYRCLHGDENSEVWRSLCARSLAEEALRTDILCNLPSYKAKIRAFQHAFSTNDCSRNVYIKKNGFTLHRNPIAQSTDGARTKIGFSEGRHAWEVWWEGPLGTVAVIGIATKRAPMQCQGYVALLGSDDQSWGWNLVDNNLLHNGEVNGSFPQCNNAPKYQIGERIRVILDMEDKTLAFERGYEFLGVAFRGLPKVCLYPAVSAVYGNTEVTLVYLGKPLDG

SEQ ID NO: 16 - protein, 717 aa, Homo sapiens
MTPPPPPPPPPGPDPAADPAADPCPWPGSLVVLFGATAGALGRDLGSDETDLILLVWQVVEPRSRQVGTLHKSLVRAEAAALSTQCREASGLSADSLARAEPLDKVLQQFSQLVNGDVALLGGGPYMLCTDGQQLLRQVLHPEASRKNLVLPDMFFSFYDLRREFHMQHPSTCPARDLTVATMAQGLGLETDATEDDFGVWEVKTMVAVILHLLKEPSSQLFSKPEVIKQKYETGPCSKADVVDSETVVRARGLPWQSSDQDVARFFKGLNVARGGVALCLNAQGRRNGEALIRFVDSEQRDLALQRHKHHMGVRYIEVYKATGEEFVKIAGGTSLEVARFLSREDQVILRLRGLPFSAGPTDVLGFLGPECPVTGGTEGLLFVRHPDGRPTGDAFALFACEELAQAALRRHKGMLGKRYIELFRSTAAEVQQVLNRYASGPLLPTLTAPLLPIPFPLAPGTGRDCVRLRGLPYTATIEDILSFLGEAAADIRPHGVHMVLNQQGRPSGDAFIQMTSAERALAAAQRCHKKVMKERYVEVVPCSTEEMSRVLMGGTLGRSGMSPPPCKLPCLSPPTYTTFQATPTLIPTETAALYPSSALLPAARVPAAPTPVAYYPGPATQLYLNYTAYYPSPPVSPTTVGYLTTPTAALASAPTSVLSQSGALVRMQGVPYTAGMKDLLSVFQAYQLPADDYTSLMPVGDPPRTVLQAPKEWVCL

SEQ ID NO: 17 - protein, 166 aa, Homo sapiens
MVANCGAAGTGGRRSCAASSVKRSASRRKHNAVRNSDVKAKGRKSRNTYTTDGGGDGDAASGAACNNATYVVWNDMNRSGRVRYDGRSKARRRGGRGDRRRDAYTRCRSRRRAVKRSRMTTWRRSVSAVYTAMDNSRHAVCYMDSASADGKRMKVSNRHDGSAYHS

SEQ ID NO: 18 - protein, 456 aa, Homo sapiens
MNSSDEEKQLQLITSLKEQAIGEYEDLRAENQKTKEKCDKIRQERDEAVKKLEEFQKISHMVIEEVNFMQNHLEIEKTCRESAEALATKLNKENKTLKRISMLYMAKLGPDVITEEINIDDEDSTTDTDGAAETCVSVQCQKQIKELRDQIVSVQEEKKILAIELENLKSKLVEVIEEVNKVKQEKTVLNSEVLEQRKVLEKCNRVSMLAVEEYEEMQVNLELEKDLRKKAESFAQEMFIEQNKLKRQSHLLLQSSIPDQQLLKALDENAKLTQQLEEERIQHQQKVKELEEQLENETLHKEIHNLKQQLELLEEDKKELELKYQNSEEKARNLKHSVDELQKRVNQSENSVPPPPPPPPPLPPPPPNPIRSLMSMIRKRSHPSGSGAKKEKATQPETTEEVTDLKRQAVEEMMDRIKKGVHLRPVNQTARPKTKPESSKGCESAVDELKGILASQ